SEQUENCE LISTING (36 sequences)

SEQ ID NO: 1 - DNA, 2682 bp, Artificial Sequence
  CDS (1)...(2682): Fusion between Aequorea victoria and human
atgcccttggtggatttcttctgcgagacctgctctaagccttggctggtgggctggtgggaccagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 893 aa, Artificial Sequence
  Fusion between Aequorea victoria and human
MPLVDFFCETCSKPWLVGWWDQFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPTWDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 3399 bp, Artificial Sequence
  CDS (1)...(3399): Fusion between Aequorea victoria and human
atggaacccccgaccgtcccctcggaaaggagcctgtctctgtcactgcccgggccccgggagggccaggccaccctgaagcctcccccgcagcacctgtggcggcagcctcggacccccatccgtatccagcagcgcggctactccgacagcgcggagcgcgccgagcgggagcggcagccgcaccggcccatagagcgcgccgatgccatggacaccagcgaccggcccggcctgcgcacgacccgcatgtcctggccctcgtccttccatggcactggcaccggcagcggcggcgcgggcggaggcagcagcaggcgcttcgaggcagagaatgggccgacaccatctcctggccgcagccccctggactcgcaggcgagcccaggactcgtgctgcacgccggggcggccaccagccagcgccgggagtccttcctgtaccgctcagacagcgactatgacatgtcacccaagaccatgtcccggaactcatcggtcaccagcgaggcgcacgctgaagacctcatcgtaacaccatttgctcaggtgctggccagcctccggagcgtccgtagcaacttctcactcctgaccaatgtgcccgttcccagtaacaagcggtccccgctgggcggccccacccctgtctgcaaggccacgctgtcagaagaaacgtgtcagcagttggcccgggagactctggaggagctggactggtgtctggagcagctggagaccatgcagacctatcgctctgtcagcgagatggcctcgcacaagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 1132 aa, Artificial Sequence
  Fusion between Aequorea victoria and human
MEPPTVPSERSLSLSLPGPREGQATLKPPPQHLWRQPRTPIRIQQRGYSDSAERAERERQPHRPIERADAMDTSDRPGLRTTRMSWPSSFHGTGTGSGGAGGGSSRRFEAENGPTPSPGRSPLDSQASPGLVLHAGAATSQRRESFLYRSDSDYDMSPKTMSRNSSVTSEAHAEDLIVTPFAQVLASLRSVRSNFSLLTNVPVPSNKRSPLGGPTPVCKATLSEETCQQLARETLEELDWCLEQLETMQTYRSVSEMASHKFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPTWDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 5 - DNA, 3375 bp, Artificial Sequence
  CDS (1)...(3375): Fusion between Aequorea victoria and human
atggaacccccgaccgtcccctcggaaaggagcctgtctctgtcactgcccgggccccgggagggccaggccaccctgaagcctcccccgcagcacctgtggcggcagcctcggacccccatccgtatccagcagcgcggctactccgacagcgcggagcgcgccgagcgggagcggcagccgcaccggcccatagagcgcgccgatgccatggacaccagcgaccggcccggcctgcgcacgacccgcatgtcctggccctcgtccttccatggcactggcaccggcagcggcggcgcgggcggaggcagcagcaggcgcttcgaggcagagaatgggccgacaccatctcctggccgcagccccctggactcgcaggcgagcccaggactcgtgctgcacgccggggcggccaccagccagcgccgggagtccttcctgtaccgctcagacagcgactatgacatgtcacccaagaccatgtcccggaactcatcggtcaccagcgaggcgcacgctgaagacctcatcgtaacaccatttgctcaggtgctggccagcctccggagcgtccgtagcaacttctcactcctgaccaatgtgcccgttcccagtaacaagcggtccccgctgggcggccccacccctgtctgcaaggccacgctgtcagaagaaacgtgtcagcagttggcccgggagactctggaggagctggactggtgtctggagcagctggagaccatgcagacctatcgctctgtcagcgagatggcctcgcacaagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaaccgcccccgcccccggtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 6 - protein, 1124 aa, Artificial Sequence [sequence omitted]

SEQ ID NO: 7 - DNA, 3399 bp, Artificial Sequence
  CDS (1)...(3399): Fusion between Aequorea victoria and human
atggaacccccgaccgtcccctcggaaaggagcctgtctctgtcactgcccgggccccgggagggccaggccaccctgaagcctcccccgcagcacctgtggcggcagcctcggacccccatccgtatccagcagcgcggctactccgacagcgcggagcgcgccgagcgggagcggcagccgcaccggcccatagagcgcgccgatgccatggacaccagcgaccggcccggcctgcgcacgacccgcatgtcctggccctcgtccttccatggcactggcaccggcagcggcggcgcgggcggaggcagcagcaggcgcttcgaggcagagaatgggccgacaccatctcctggccgcagccccctggactcgcaggcgagcccaggactcgtgctgcacgccggggcggccaccagccagcgccgggagtccttcctgtaccgctcagacagcgactatgacatgtcacccaagaccatgtcccggaactcatcggtcaccagcgaggcgcacgctgaagacctcatcgtaacaccatttgctcaggtgctggccagcctccggagcgtccgtagcaacttctcactcctgaccaatgtgcccgttcccagtaacaagcggtccccgctgggcggccccacccctgtctgcaaggccacgctgtcagaagaaacgtgtcagcagttggcccgggagactctggaggagctggactggtgtctggagcagctggagaccatgcagacctatcgctctgtcagcgagatggcctcgcacaagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgaaaatcacaacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 8 - protein, 1132 aa, Artificial Sequence [sequence omitted]

SEQ ID NO: 9 - DNA, 32 bp, Artificial Sequence
  Primer sequence
gtaagcttaagatgcccttggtggatttcttc

SEQ ID NO: 10 - DNA, 28 bp, Artificial Sequence
  Primer sequence
gtggatcccaggtagggtctccacctga

SEQ ID NO: 11 - DNA, 28 bp, Artificial Sequence
  Primer sequence
gtttaaaaggatgttgaaccgtgagctc

SEQ ID NO: 12 - DNA, 28 bp, Artificial Sequence
  Primer sequence
gtggatcccaggtagggtctccacctga

SEQ ID NO: 13 - DNA, 28 bp, Artificial Sequence
  Primer sequence
gtaagcttgcgccatggaacccccgacc

SEQ ID NO: 14 - DNA, 33 bp, Artificial Sequence
  Primer sequence
ggttttaaacttgtgcgaggccatctcgctgac

SEQ ID NO: 15 - DNA, 49 bp, Artificial Sequence
  Primer sequence
gatgagtcggtgctcgaaaatcacaacctggccgtgggcttcaagctgc

SEQ ID NO: 16 - DNA, 49 bp, Artificial Sequence
  Primer sequence
gcagcttgaagcccacggccaggttgtgattttcgagcaccgactcatc

SEQ ID NO: 17 - DNA, 70 bp, Artificial Sequence
  Primer sequence
cccatcacccacgatgaaggaacgagaaaaacagcaaccgcccccgcccccggtaccacacttacagccc

SEQ ID NO: 18 - DNA, 70 bp, Artificial Sequence
  Primer sequence
gggctgtaagtgtggtaccgggggcgggggcggttgctgtttttctcgttccttcatcgtgggtgatggg

SEQ ID NO: 19 - DNA, 36 bp, Artificial Sequence
  Primer sequence
ccggaattccgccatggaacccccgaccgtcccctc

SEQ ID NO: 20 - DNA, 45 bp, Artificial Sequence
  Primer sequence
ggcaagctttttcaaccctgtgatttgggacatgggctgtaagtg

SEQ ID NO: 21 - DNA, 53 bp, Artificial Sequence
  Primer sequence
ggcaagcttatgcacagctctagtctgactaattcaagtatcccaaggtttgg

SEQ ID NO: 22 - DNA, 49 bp, Artificial Sequence
  Primer sequence
gccccgcggcgtgtcaggagaacgatcatctatgacacaggcttcaggc

SEQ ID NO: 23 - DNA, 3090 bp, Artificial Sequence
  Fusion between Aequorea victoria and human
atggaacccccgaccgtcccctcggaaaggagcctgtctctgtcactgcccgggccccgggagggccaggccaccctgaagcctcccccgcagcacctgtggcggcagcctcggacccccatccgtatccagcagcgcggctactccgacagcgcggagcgcgccgagcgggagcggcagccgcaccggcccatagagcgcgccgatgccatggacaccagcgaccggcccggcctgcgcacgacccgcatgtcctggccctcgtccttccatggcactggcaccggcagcggcggcgcgggcggaggcagcagcaggcgcttcgaggcagagaatgggccgacaccatctcctggccgcagccccctggactcgcaggcgagcccaggactcgtgctgcacgccggggcggccaccagccagcgccgggagtccttcctgtaccgctcagacagcgactatgacatgtcacccaagaccatgtcccggaactcatcggtcaccagcgaggcgcacgctgaagacctcatcgtaacaccatttgctcaggtgctggccagcctccggagcgtccgtagcaacttctcactcctgaccaatgtgcccgttcccagtaacaagcggtccccgctgggcggccccacccctgtctgcaaggccacgctgtcagaagaaacgtgtcagcagttggcccgggagactctggaggagctggactggtgtctggagcagctggagaccatgcagacctatcgctctgtcagcgagatggcctcgcacaagttcaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagcttatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 24 - protein, 1029 aa, Artificial Sequence
  Fusion between Aequorea victoria and human
MEPPTVPSERSLSLSLPGPREGQATLKPPPQHLWRQPRTPIRIQQRGYSDSAERAERERQPHRPIERADAMDTSDRPGLRTTRMSWPSSFHGTGTGSGGAGGGSSRRFEAENGPTPSPGRSPLDSQASPGLVLHAGAATSQRRESFLYRSDSDYDMSPKTMSRNSSVTSEAHAEDLIVTPFAQVLASLRSVRSNFSLLTNVPVPSNKRSPLGGPTPVCKATLSEETCQQLARETLEELDWCLEQLETMQTYRSVSEMASHKFKRMLNRELTHLSEMSRSGNQVSEYISTTFLDKQNEVEIPSPTMKEREKQQAPRPRPSQPPPPPVPHLQPMSQITGLKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDTPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 25 - DNA, 2790 bp, Artificial Sequence
  Fusion between Aequorea victoria and human
atgatgcacgtgaataattttccctttagaaggcattcctggatatgttttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 26 - protein, 929 aa, Artificial Sequence
  Fusion between Aequorea victoria and human
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDTRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 27 - DNA, 29 bp, Artificial Sequence
  Primer sequence
gtaagcttgcgaacatgatgcacgtgaat

SEQ ID NO: 28 - DNA, 29 bp, Artificial Sequence
  Primer sequence
gtgaattcccgtgtcaggagaacgatcat

SEQ ID NO: 29 - DNA, 2682 bp, Artificial Sequence
  Fusion between Aequorea victoria and human
atgcccttggtggatttcttctgcgagacctgctctaagccttggctggtgggctggtgggaccagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacactagtgaccaccctgtcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctcctagggttcgtgaccgccgccgggatcactctcggcatggacgagctgtacaagtaa

SEQ ID NO: 30 - protein, 893 aa, Artificial Sequence
  Fusion between Aequorea victoria and human
MPLVDFFCETCSKPWLVGWWDQFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPTWDPPVATMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLGFVTAAGITLGMDELYK

SEQ ID NO: 31 - DNA, 3399 bp, Artificial Sequence
  Fusion between Aequorea victoria and human
atggaacccccgaccgtcccctcggaaaggagcctgtctctgtcactgcccgggccccgggagggccaggccaccctgaagcctcccccgcagcacctgtggcggcagcctcggacccccatccgtatccagcagcgcggctactccgacagcgcggagcgcgccgagcgggagcggcagccgcaccggcccatagagcgcgccgatgccatggacaccagcgaccggcccggcctgcgcacgacccgcatgtcctggccctcgtccttccatggcactggcaccggcagcggcggcgcgggcggaggcagcagcaggcgcttcgaggcagagaatgggccgacaccatctcctggccgcagccccctggactcgcaggcgagcccaggactcgtgctgcacgccggggcggccaccagccagcgccgggagtccttcctgtaccgctcagacagcgactatgacatgtcacccaagaccatgtcccggaactcatcggtcaccagcgaggcgcacgctgaagacctcatcgtaacaccatttgctcaggtgctggccagcctccggagcgtccgtagcaacttctcactcctgaccaatgtgcccgttcccagtaacaagcggtccccgctgggcggccccacccctgtctgcaaggccacgctgtcagaagaaacgtgtcagcagttggcccgggagactctggaggagctggactggtgtctggagcagctggagaccatgcagacctatcgctctgtcagcgagatggcctcgcacaagtttaaaaggatgttgaaccgtgagctcacacacctgtcagaaatgagcaggtccggaaaccaggtctcagagtacatttccacaacattcctggacaaacagaatgaagtggagatcccatcacccacgatgaaggaacgagaaaaacagcaagcgccgcgaccaagaccctcccagccgcccccgccccctgtaccacacttacagcccatgtcccaaatcacagggttgaaaaagttgatgcatagtaacagcctgaacaactctaacattccccgatttggggtgaagaccgatcaagaagagctcctggcccaagaactggagaacctgaacaagtggggcctgaacatcttttgcgtgtcggattacgctggaggccgctcactcacctgcatcatgtacatgatattccaggagcgggacctgctgaagaaattccgcatcccggtggacacgatggtgacatacatgctgacgctggaggatcactaccacgctgacgtggcctaccataacagcctgcacgcagctgacgtgctgcagtccacccacgtactgctggccacgcctgcactagatgcagtgttcacggacctggagattctcgccgccctcttcgcggctgccatccacgatgtggatcaccctggggtctccaaccagttcctcatcaacaccaattcggagctggcgctcatgtacaacgatgagtcggtgctcgagaatcaccacctggccgtgggcttcaagctgctgcaggaggacaactgcgacatcttccagaacctcagcaagcgccagcggcagagcctacgcaagatggtcatcgacatggtgctggccacggacatgtccaagcacatgaccctcctggctgacctgaagaccatggtggagaccaagaaagtgaccagctcaggggtcctcctgctagataactactccgaccgcatccaggtcctccggaacatggtgcactgtgccgacctcagcaaccccaccaagccgctggagctgtaccgccagtggacagaccgcatcatggccgagttcttccagcagggtgaccgagagcgcgagcgtggcatggaaatcagccccatgtgtgacaagcacactgcctccgtggagaagtctcaggtgggttttattgactacattgtgcacccattgtgggagacctgggcggaccttgtccacccagatgcccaggagatcttggacactttggaggacaaccgggactggtactacagcgccatccggcagagcccatctccgccacccgaggaggagtcaagggggccaggccacccacccctgcctgacaagttccagtttgagctgacgctggaggaggaagaggaggaagaaatatcaatggcccagataccgtgcacagcccaagaggcattgactgcgcagggattgtcaggagtcgaggaagctctggatgcaaccatagcctgggaggcatccccggcccaggagtcgttggaagttatggcacaggaagcatccctggaggccgagctggaggcagtgtatttgacacagcaggcacagtccacaggcagtgcacctgtggctccggatgagttctcgtcccgggaggaattcgtggttgctgtaagccacagcagcccctctgccctggctcttcaaagcccccttctccctgcttggaggaccctgtctgtttcagagcatgccccgggcctcccgggcctcccctccacggcggccgaggtggaggcccaacgagagcaccaggctgccaagagggcttgcagtgcctgcgcagggacatttggggaggacacatccgcactcccagctcctggtggcggggggtcaggtggagaccctacctgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacactagtgaccaccctgtcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctcctagggttcgtgaccgccgccgggatcactctcggcatggacgagctgtacaagtaa

SEQ ID NO: 32 - protein, 1132 aa, Artificial Sequence
  Fusion between Aequorea victoria and human
MEPPTVPSERSLSLSLPGPREGQATLKPPPQHLWRQPRTPIRIQQRGYSDSAERAERERQPHRPIERADAMDTSDRPGLRTTRMSWPSSFHGTGTGSGGAGGGSSRRFEAENGPTPSPGRSPLDSQASPGLVLHAGAATSQRRESFLYRSDSDYDMSPKTMSRNSSVTSEAHAEDLIVTPFAQVLASLRSVRSNFSLLTNVPVPSNKRSPLGGPTPVCKATLSEETCQQLARETLEELDWCLEQLETMQTYRSVSEMASHKFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPTWDPPVATMVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLGFVTAAGITLGMDELYK

SEQ ID NO: 33 - DNA, 33 bp, Artificial Sequence
  Primer sequence
tgtactagtgaccaccctgtcttacggcgtgca

SEQ ID NO: 34 - DNA, 30 bp, Artificial Sequence
  Primer sequence
ctgactagtgtgggccagggcacgggcagc

SEQ ID NO: 35 - DNA, 42 bp, Artificial Sequence
  Primer sequence
cccggcggcggtcacgaaccctaggaggaccatgtgatcgcg

SEQ ID NO: 36 - DNA, 42 bp, Artificial Sequence
  Primer sequence
cgcgatcacatggtcctcctagggttcgtgaccgccgccggg

(19) United States
(12) Patent Application Publication
(10) Pub. No.: US 2003/0187056 A1
(43) Pub. Date: Oct. 2, 2003

(54) Live cell procedures to identify to identify compounds modulating intracellular distribution of phosphodiesterase( pde) enzymes

(76) Inventors: Bernard Robert Terry, Frederiksberg (DK); Kurt Marshall Scudder, Virum (DK); Sara Petersen Bjorn, Lyngby (DK); Ole Thastrup, Birkerod (DK); Dorthe Christensen Almholt, Greve (DK); Morten Praestegaard, Ballerup (DK)

Correspondence Address: BIRCH STEWART KOLASCH & BIRCH, PO BOX 747, FALLS CHURCH, VA, 22040-0747, US

(21) Appl. No.: 10/257,909

(22) PCT Filed: Apr. 11, 2001
(86) PCT No.: PCT/DK01/00264

(30) Foreign Application Priority Data
Apr. 17, 2000 (DK) PA 2000 00651
May 29, 2000 (DK) PA 2000 00849

(51) Int. Cl. A61K 31/4015

(52) U.S. Cl. 514/424

(57) ABSTRACT

An alternative therapeutic approach for PDE4 inhibition is disclosed. PDE4 dislocators, will remove the PDE4 away from the native location in the cell and thereby increase the concentration of cAMP in this location. By dislocating the PDE4, and thereby not acting directly on the catalytic, among phosphodiesterase inhibitors, well conserved site, the compound will act e.g. at the binding domain of the PDE4, thereby providing isoform-specific ‘inhibitors’ of PDE4. The dislocation of PDE4s are visualised with fusions to GFP. The native location is induced by treatment with Rolipram.

### BACKGROUND

Cyclic AMP is a ubiquitous second messenger. It is generated through the action of adenylyl cyclase and serves to transduce the action of many hormones, neurotransmitters and other cellular effectors. cAMP exerts its effects on cells through its ability to bind to specific intracellular regulatory proteins. These are protein kinase A (PKA), cyclic nucleotide gated ion channels (CNG channels) and cyclic AMP stimulated GTPase exchange factors (cAMP-GEFs, EPACs). Such effectors allow cAMP to regulate cellular processes in a cell-type specific fashion. Thus elevated cAMP levels can for example affect CNS function (e.g. depression), cardiovascular function, inflammatory cells/immune system, cell adhesion and metabolic processes. These actions, however, depend upon cAMP being elevated not only in particular cell types but also in particular intracellular locations (Houslay and Milligan 1997).

The only way to degrade cAMP is through the action of cyclic nucleotide phosphodiesterases (PDEs) (Conti and Jin 1999). These hydrolyse 3′,5′ cyclic adenosine monophosphate (cAMP) to 5′-adenosine monophosphate (AMP). It is now well-recognised that a large multi-gene family encode PDEs. However, only certain of these enzymes are capable of hydrolysing cAMP. These are members of the PDE1, PDE2, PDE3, PDE4, PDE7, PDE8 and PDE11 families. Selective inhibitors have been generated against certain of these families, e.g. PDE3 and PDE4 enzymes. These inhibitors are targeted to the enzyme catalytic unit, being identified through screens which looked for inhibition of cAMP hydrolysis. Such inhibitors thus display competitive kinetics of inhibition. PDE3 and PDE4 selective inhibitors have, for example, been shown to generate clearly distinct pharmacological responses. For example, the PDE3 inhibitor, milrinone serves as positive inotropic agent and increase the force of contraction of the heart, whereas the PDE4 inhibitor, Rolipram does not (Manganiello et al. 1995). In contrast to this, PDE4 inhibitors (Rolipram, Ariflo®) can inhibit the action of many cells of haematopoiec origin that are associated with inflammatory responses and may also exert antidepressant effects (Rolipram), whereas PDE3 inhibitors (milrinone, cilostamide) do not. Whilst PDE isoenzymes show cell-type specific patterns of expression, PDE3 and PDE4 enzymes are often found in many types of cells, including cardiac myocytes and inflammatory cells. Thus the ability of PDE isoenzyme selective inhibitors to exert very different effects on a particular cell type is not necessarily due to lack of expression of one or other PDE isoenzyme in that cell type. Of course, in certain instances apparent selectivity can arise due to very different levels of PDE3 and PDE4 enzyme activity in cells where different actions of PDE isoenzyme-selective inhibitors have been noted. However, in many instances this is simply not the case. To explain this apparent dichotomy the concept of compartmentalised cAMP signalling has arisen. This envisages that cAMP is not uniformly distributed through the cell interior. Indeed, there is direct evidence which demonstrates this (Hempel et al. 1996).

As cAMP is only degraded by cAMP-PDEs in cells, then the attenuation of their activity can be expected to lead to an increase in cAMP levels and the triggering of a cellular response. As PDE3 and PDE4 enzymes are localised to discrete intracellular sites then they can be expected to control ‘localised pools’ of cAMP that, in turn, may control the activity of restricted PKA-RII/EPAC/CNG channels. Traditional approaches have focussed entirely on developing active site-directed selective PDE inhibitors to provide novel therapeutic agents (Souness and Rao 1997). However, the realisation (Houslay et al. 1998) that specific PDE4 isoforms show precise intracellular targeting offers a radically new means of altering PDE functioning in intact cells and generating a novel class of therapeutic agents affecting PDE functioning. This exploits an ability to disrupt the intracellular targeting of specific PDE4 isoenzymes and thus to remove the target isoenzyme from its functionally relevant intracellular compartment. Such a relocalisation would be expected to elevate intracellular cAMP levels in a particular subcellular location (‘compartment’) and lead to activation of PKA/EPAC/CNG channels in the vicinity. This offers the potential of generating isoform-specific ‘inhibitors’ that rather than acting on the enzyme catalytic unit serve to displace the target PDE4 isoforms from its functional relevant (anchor) site within a cell. This may involve the release of the enzyme into the cytosol where it will be grossly diluted or re-targeted.

The PDE4 enzyme family is a family where active-site directed inhibitors have anti-depressant and anti-inflammatory action. PDE4 isoforms show distinct and cell-type specific patterns of expression (Houslay et al. 1998). In addition, within cells PDE4 isoforms also exhibit highly specific intracellular distribution patterns. Thus, for example, the PDE4A1 isoform appears to be expressed only in within certain brain regions (Houslay et al. 1998). Indeed, when PDE4A1 is expressed in various cell types it shows a distinctive pattern of intracellular distribution, implying targeted association (Pooley et al. 1997).

PDE4 enzymes encoded by four distinct genes (Houslay et al. 1998), specifically hydrolyse cAMP. The large family of PDE4 isoforms arise through the use of alternative promoters and alternative mRNA splicing.

The recruitment or re-localisation of proteins plays a major role in many key signalling systems. This is evident in (i) the activation of protein kinase C (PKC), where recruitment to the plasma membrane is an inherent part of the activation process of this enzyme; (ii) the activation of p42/44 MAP Kinase depends on multiple proteins whose transfer from cytosolic to membrane compartments has a central role and (iii) the cAMP-driven relocalisation of rap1 is crucial to its activation. Thus there is a need for the ability to detect the localisation and any re-localisation of PDE4 enzymes in intact, living cells, as such detection is expected to provide a novel and innovative means for identifying new therapeutic agents.

Present screening assays for compounds interfering with the activity of PDE enzymes are based upon variations of methods which assess PDE catalytic activity; namely the ability of the enzyme to hydrolyse 3′5′ cyclic adenosine monophosphate (cAMP) to 5′-AMP. This is usually performed in multiwell format using detection of cAMP hydrolysis using proximity-based radionuclide assays. Such screens detect compounds that alter catalytic activity. To date these have identified compounds that bind to the catalytic site as competitive inhibitors. Thus all compounds reported on to date bind to the catalytic site and thus compete with the substrate cAMP for binding. The enzymes used in these screens often are cell extracts of endogenous PDE4 enzymes that have been partially purified to remove non-PDE4 enzymes. These suffer from the fact that they may be contaminated by as yet unknown PDE species and that they will contain mixtures of PDE4 isoforms. An alternative approach has been to use recombinant enzymes in screens where expression has been done in various of cell lines/systems such as sf9 cells, S. cerevisiae, E. coli and transfected mammalian cell lines. This allows for isoform specific analyses to be done. However, as the catalytic unit of PDE4 enzymes is identical for isoforms within each PDE4 subfamily then it is near impossible to conceive that an isoform selective inhibitor could be identified through such analyses. In addition, the catalytic subunit is highly conserved within the enzymes of the 4 gene PDE4 family itself. This means that it is likely to be extremely difficult, although not inconceivable, to obtain inhibitors that are highly selective between each of the four families. To date, the best selectivity reported is that for Ariflo® which shows some 8 to 10-fold selectivity for the PDE4D family over enzymes from the other three PDE4 families (Barnette et al. 1998). There is thus a need to develop strategies that will allow for the identification of compounds that serve as isoform-specific ‘inhibitors’. There is therefore a need for procedures that can (i) allow for the rapid screening of agents that disrupt the targeting of PDE4 isoforms in living cells and (ii) identify conformationally distinct forms of PDE4 living cells. Procedures that allow for these aims to be achieved can be expected to lead to the development of novel therapeutics. In addition they will lead to the provision of diagnostic aids to identify compounds exerting conformationally distinct effects on PDE4 enzymes and thus as being of use in compound development to either screen for beneficial e.g. (anti-inflammatory, anti-depressant) or against adverse effects (e.g nausea, vomiting, arteritis).

### SUMMARY OF THE INVENTION

The examples in the application disclose, for the first time, that the selective PDE4 inhibitor Rolipram affects the physical properties and behaviour of PDE4A4 such that the general cytoplasmic distribution of PDE4A4 in most cells gradually changes to one consisting of concentrations of PDE4A4 located at several distinct spots within the cytoplasm (example 3). Pre-treatment of the cells with cycloheximide, a protein synthesis inhibitor, prevents formation of spots induced by Rolipram, indicating that protein synthesis is a necessary part of the observed spot formation. Once spots have formed, removal of Rolipram results in their rapid dissolution. However, replacement of Rolipram causes the spots to rapidly reform. This is the first evidence that binding of Rolipram induce changes in distribution.

Additionally, example 15 discloses, also for the first time, that Rolipram affects the physical properties and behaviour of PDE4A1 but in a way that is very different to the effects this compound has on the behaviour of PDE4A4. PDE4A1 accumulates as small perinuclear spots in otherwise untreated cells, and treatment with Rolipram causes these spots to disperse into the cytoplasm. Subsequent removal of Rolipram results in the rapid re-appearance of perinuclear spots.

Rolipram causes a change in the distribution of probes based on both PDE4A4 and PDE4A1. The non PDE4-specific inhibitors of cyclic nucleotide phosphodiesterases such as trequinsin, etazolate, milrinone, zaprinast, caffeine, theophylline and cilostamide cause no redistribution of the PDE4A probes, even at physiologically very high concentrations, whereas the PDE4-specific inhibitors Denbufylline (BRL30892), RS25344, and Ro 20-1724 produce changes in the distribution of these probes which are indistinguishable from those induced by Rolipram treatment (examples 5, 6, 15 and 16). Piclamilast (RP73401), also a highly potent and specific PDE4 inhibitor, induces no redistribution of the PDE4A probes. However, RP73401 will prevent the redistribution that normally is caused by the presence of Rolipram (examples 9 and 17). Thus, only certain classes of PDE inhibitors cause intracellular redistribution of the PDE4A probes, and these are all specific inhibitors of PDE4 enzymes (such as Rolipram); certain other PDE inhibitors are unable to cause the intracellular redistribution of PDE4A, but are able to compete with, reverse or prevent the action of compounds that cause redistribution, and these are also PDE4-specific inhibitors (the non PDE4-specific inhibitors are not able to reverse or inhibit the intracellular redistribution of PDE4A caused by, for example, Rolipram). The induction of redistribution of the PDE4A probes by a certain class of PDE4-specific inhibitors is a new and inventive finding of a concept of reverse interaction between different regions of the PDE4A protein; the impulse for redistribution originates at the catalytic cleft of the PDE4A where Rolipram binds, and effects a critical switch-like change at some other domain that anchors the enzymes in position within the cell. This finding immediately provides a screen for compounds that induce the intracellular redistribution of PDE4As (agonists) from those that prevent the induction of redistribution (antagonists) and a screen for those compounds that can antagonise this induced redistribution.

### DETAILED DISCLOSURE OF THE INVENTION

In this deposition, we propose a new method to screen for compounds that can disrupt the intracellular targeting of specific PDE4 isoenzymes/isoforms and that either detect or generate specific conformational states of PDE4 isoforms which either lead to alterations in or define intracellular distribution.

In summary, Rolipram and certain other PDE4 inhibitors affect the distribution of at least two isoforms of PDE4. Compounds with the same effect as Rolipram on the distribution of these PDE4 isoforms also share certain other properties:

1) They are all PDE4 inhibitors, although with very wide ranging affinities, from Ro 20-1724 with an IC₅₀ against PDE4 of 2.4 μM (Souness and Rao,1997) to RS25344 with IC₅₀ of 0.28 nM (Saldou et al., 1998). These compounds are termed specific to PDE4, since they inhibit other PDEs either weakly or not at all. The inverse ratio between IC₅₀ of a compound to PDE4s versus its inhibition of other cAMP-degrading PDEs, such as PDE3s is often used as a measure of specificity. For example, (IC₅₀ PDE3)/(IC₅₀ PDE4) values are >2,200 for Rolipram, 1,170,000 for RS25344 but 0.00041 for Trequinsin, clearly marking Rolipram and RS25344 highly specific for PDE4s, whereas Treqinsin is a more specific inhibitor of PDE3 enzymes.

2) They are all known to be able to displace tritiated Rolipram from what has commonly been referred to as the high affinity Rolipram binding site (HARBS), usually assayed using microsomal vesicles obtained from brain—see Souness and Rao (1997). It is widely accepted that compounds having affinity for this site are associated with certain pharmacologies and physiological effects in animals, of which some are beneficial, but others are troublesome and may be characterised as undesirable side-effects, such as headaches, nausea and emesis.

3) The compounds that change the distribution of the PDE4A probes all have a relatively lower affinity for the “cAMP binding site” in PDE4s than they do for the HARBS, so that the ratio of (IC₅₀ PDE4)/(Kᵢ for HARBS) gives a high score for Rolipram-like compounds, and low scores for those compounds that do not cause changes in PDE4 distribution (see Table 1).

TABLE 1

| | | Displacement of | |
|---|---|---|---|
| | PDE4 inhibition | [³H] Rolipram | |
| Compound | (IC₅₀: μM) | (ᵢ: μM) | PDE4/HARBS |
| (±) Rolipram | 0.31 | 0.0017 | 238 |
| Ro 20-1724 | 2.4 | 0.017 | 141 |
| Denbufylline | 0.20 | 0.0041 | 49 |
| IBMX | 14 | 0.84 | 17 |
| RP73401 | 0.0012 | 0.0004 | 3 |
| Trequinsin | 0.4 | 1.7 | 023 |

(Data from Souness and Rao, 1997)

As illustrated in Table 2, the assay of the present invention will inter alia identify sharing the Rolipram antidepressent and/or anti-inflammatory properties, without inducing emesis.

TABLE 2

| | | | | | | | Reversal |
|---|---|---|---|---|---|---|---|
| | | | | | Removal | | of |
| | | | | | of | | Rolipram- |
| | | | Inhibition | Formation | Rolipram- | Removal | induced |
| | | | of PDE4 | of | induced | of | removal of |
| | | | catalytic | PDE4A4 | PDE4A4- | PDE4A1 | PDE4A1 |
| Drug name | Clinical effect | Emesis | activity | spots | spots | spots | spots |
| Rolipram | antidepressant | yes | yes | yes | — | yes | — |
| | anti- | | | | | | |
| | inflammatory | | | | | | |
| Denbufylline | anti- | yes | yes | yes | no | yes | no |
| (BRL30892) | inflammatory | | | | | | |
| RS25344 | anti- | yes | yes | yes | no | yes | no |
| | inflammatory | | | | | | |
| RO 20-1724 | anti- | yes | yes | yes | no | yes | no |
| | inflammatory | | | | | | |
| Piclamilast | anti- | no | yes | no | yes | no | yes |
| (RP73401) | inflammatory | | | | | | |
| Ariflo ® | anti- | no | yes | no | yes | no | yes |
| | inflammatory | | | | | | |

One aspect of the present invention is the number and type of uses to which this observation can be put. Examples of such uses are:

1) Screening of potential or newly discovered PDE4 inhibitors for Rolipram-like properties. Such a screen may be most useful as a counterscreen, for the detection of compounds which cause undesirable side effects, such as emesis, nausea, headaches, and excess gastric acidity.

2) Screening for potential or newly discovered PDE4 inhibitors with the ability to reverse or prevent the change in PDE4 distribution caused by Rolipram. Such compounds should be useful inhibitors of PDE4 but lack Rolipram-like properties, such as emesis, nausea, headaches, and excess gastric acidity.

3) Screening for potential inhibitors of PDE4 cellular activity that have a defined and novel mode of action, inhibitors which work by dislocating specific PDE4 isoforms from their normal sites within cells thereby modulating their effectiveness to function in cellular signalling.

The uses can be applied also to gene families B, C and D of PDE4. Uses 1 and 2 can be applied e.g. through exchange of their catalytic domains into the structure of PDE4As. Such hybrid probes, when expressed in cells, bind Rolipram and other PDE4-specific inhibitors with affinities reflecting the particular properties of the substitute catalytic site (of PDE4B in the example above), but exhibit the redistribution behaviour of the chosen PDE4A. Measurement of the redistribution behaviour reflects the binding properties specific to the imported catalytic site.

Uses 1 and 2 may also be applied rather generally to any PDE families and subfamilies thereof that do not belong to the class of PDE4 enzymes, through construction of hybrid probes between a PDE4A and the catalytic domain of the chosen PDE type in the manner described. However with these mixed class hybrids, the choice of Rolipram-like reference compound, which for PDE4s may be Rolipram, will be made from those that are known to be specific inhibitors of the particular PDE class which contributes the catalytic domain to the hybrid probe.

Thus, one aspect of the invention relates to a method to monitor changes in intracellular distribution of phosphodiesterases of subtype 4 (PDE4s) in living cells, the method comprising the steps of:

(a) recording the intracellular distribution of the PDE4;

(b) adding a Rolipram-like reference compound to the cells in (a) or to similar cells, the Rolipram-like compound being able to bind to the catalytic cleft of the PDE4, or to some other part of the enzyme or an associated protein whereby it induces redistribution of the PDE4 probe;

(c) recording the intracellular distribution of the PDE4 in the cells in step (b);

(d) determining the effect on the intracellular distribution of the PDE4 of the Rolipram-like reference compound by comparing the intracellular distribution recorded in step (a) with the intracellular distribution recorded in step (c).

In another aspect of the invention inclusion of multiple cell types allows tissue specific characterisation of specific PDE4 isoforms and/or mutations thereof.

In the present invention phosphodiesterases of class 4 (PDE4s) should be understood as enzymes which are inhibited by Rolipram with an IC₅₀ of less than 5 μM; enzymes capable of reacting in this specific way being selected from the list of all protein products (including all splice variants derived from) the genes designated PDE4A, PDE4B, PDE4C and PDE4D. Throughout this application the PDE4 is selected from this group.

In the present invention the intracellular distribution should be understood as the distribution of a gene product within the volume of the cell. More specifically, how it is disposed relative to other identifiable cellular features or compartments, or organelles such as the plama membrane, the Golgi membranes, endosomal vesicles, nucleus, endoplasmic reticulum, mitochondria and so on. As such, the intracellular distribution indicates possible direct association with such features, or at least with components that themselves are associated in some way with those features. It should be noted that a distinctive non-homogenous distribution may be maintained not only through static anchoring or tethering, but may also be maintained through dynamic interchange, where the rates of association and dissociation favour the state of association. In this context location, position, localisation and distribution can be used interchangeably. In the invention the cell and/or cells are mechanically intact and alive throughout the experiment. In another embodiment of the invention, the cell or cells is/are fixed at a point in time after the application of the influence at which the response has been predetermined to be significant, and the recording is made at an arbitrary later time.

The mechanically intact living cell or cells could be selected from the group consisting of fungal cells, such as a yeast cells; invertebrate cells including insect cells; and vertebrate cells, such as mammalian cells. These cells are incubated at a temperature of 30° C. or above, preferably at a temperature of from 32° C. to 39° C., more preferably at a temperature of from 35° C. to 38° C., and most preferably at a temperature of about 37° C. during the time period over which the influence is observed. In one aspect of the invention the mechanically intact living cell is part of a matrix of identical or non-identical cells.

A cell used in the present invention may contain a nucleic acid construct encoding a fusion polypeptide as defined herein and be capable of expressing the sequence encoded by the construct. The cell is a eukaryotic cell selected from the group consisting of fungal cells, such as yeast cells; invertebrate cells including insect cells; vertebrate cells such as mammalian cells. The preferred cells are mammalian cells.

The term “mammalian cell” is intended to indicate any living cell of mammalian origin. The cell may be an established cell line, many of which are available from The American Type Culture Collection (ATCC, Virginia, USA) or a primary cell with a limited life span derived from a mammalian tissue, including tissues derived from a transgenic animal, or a newly established immortal cell line derived from a mammalian tissue including transgenic tissues, or a hybrid cell or cell line derived by fusing different celltypes of mammalian origin e.g. hybridoma cell lines. The cells may optionally express one or more non-native gene products, e.g. receptors, enzymes, enzyme substrates, prior to or in addition to the fluorescent probe. Preferred cell lines include but are not limited to those of fibroblast origin, e.g. BHK, CHO, BALB, NIH-3T3 or of endothelial origin, e.g. HUVEC, BAE (bovine artery endothelial), CPAE (cow pulmonary artery endothelial), HLMVEC (human lung microvascular endothelial cells), or of airway epithelial origin, e.g. BEAS-2B, or of pancreatic origin, e.g. RIN, INS-1, MIN6, bTC3, aTC6, bTC6, HIT, or of hematopoietic origin, e.g.primary isolated human monocytes, macrophages, neutrophils, basophils, eosinophils and lymphocyte populations, AML-14, AML-193, HL-60, RBL-1, U937, RAW, JAWS, or of adipocyte origin, e.g. 3T3-L1, human pre-adipocytes, or of neuroendocrine origin, e.g. AtT20, PC12, GH3, muscle origin, e.g. SKMC, A10, C2C12, renal origin, e.g. HEK 293, LLC-PK1, or of neuronal origin, e.g. SK-N-DZ, SK-N-BE(2), HCN-1A, NT2/D1.

The examples of the present invention is based on CHO cells. Therefore fibroblast derived cell lines such as BALB, NIH-3T3 and BHK cells are preferred.

In another aspect of the invention the cells could be from an organism carrying in at least one of its component cells a nucleic acid sequence encoding a fusion polypeptide as defined herein and be capable of expressing said nucleic acid sequence. The organism is selected from the group consisting of unicellular and multicellular organisms, such as a mammal.

Recording the intracellular distribution of the PDE4 in cells can be performed in numerous ways, known to the person skilled in the art. One example is antibody staining of the PDE4s wherein antibodies are raised essentially as described by Shakur et al. (1995); the cells are treated and stained essentially as described by Pooley et al. (1997). In raising the antisera, it is desirable to use isoform-specific epitopes in order to allow the distribution of specific PDE4 isoforms to be identified and recorded. The four families of PDE4s can be indvidually recognised using antisera raised against peptides that copy all or part of the C-terminal protein sequences that are unique to each family. Individual PDE4 isoforms may be recognised by antisera raised in the same way against the unique N-terminal portions of these enzymes.

A preferred way of recording the intracellular distribution of the PDE4 in the cells is by, prior to the initial recording, constructing a probe allowing the location of the PDE4 to be recorded and subsequently transfecting cells with the constructed probe.

Throughout this application a probe must be understood as a nucleotide sequence genetically encoding an identifiable protein comprising the PDE4 or part thereof.

Identification of the probe protein in a way which allows the location of the PDE4 to be recorded can be performed in several ways. Examples are:

immunodetection, wherein an engineered antigenic tag is incorporated into the probe, such as the “flag” or “myc” tags which are foreign and therefore unique antigens within mammalian cells, and for which mass-produced antibodies are available so that the need to develop an antibody to each new probe produced is avoided.

direct detection, wherein the probe is engineered to include a protein sequence able to trap or chelate a luminophore, or to breakdown luciferin and thereby generate light, or to convert a substrate to a coloured product thereby directly revealing its own cellular distribution.

A preferred method of recording the localisation of the PDE4 is by fluorescence detection, wherein the probe is a fusion of a luminophore and a PDE4, wherein the luminophore encodes a fluorescent protein such as the fluorophore GFP. Using fluorescence detection methods the distribution of GFP can be visualised continuously.

In the present context, the term “green fluorescent protein” (GFP) is intended to indicate a protein which, when expressed by a cell, emits fluorescence upon exposure to light of the correct excitation wavelength (e.g. as described by Chalfie, M. et al. (1994) Science 263, 802-805). Such a fluorescent protein in which one or more amino acids have been substituted, inserted or deleted is also termed “GFP”. “GFP” as used herein includes wild-type GFP derived from the jelly fish Aequorea victoria, or from other members of the Coelenterata, such as the red fluorescent protein from Discosoma sp. (Matz, M. V. et al. 1999, Nature Biotechnology 17: 969-973) or fluorescent proteins from other animals, fungi or plants, and modifications of GFP, such as the blue fluorescent variant of GFP disclosed by Heim et al. (Heim, R. et al., 1994, Proc.Natl.Acad.Sci. 91:26, pp 12501-12504), and other modifications that change the spectral properties of the GFP fluorescence, or modifications that exhibit increased fluorescence when expressed in cells at a temperature above about 30° C. described in PCT/DK96/00051, published as WO 97/11094 on Mar. 27, 1997 and hereby incorporated by reference, and which comprises a fluorescent protein derived from Aequorea Green Fluorescent Protein or any functional analogue thereof, wherein the amino acid in position 1 upstream from the chromophore has been mutated to provide an increase of fluorescence intensity when the fluorescent protein of the invention is expressed in cells. Preferred GFP variants are F64L-GFP, F64L-Y66H-GFP F64L-S65T-GFP, F64L-E222G-GFP. One especially preferred variant of GFP for use in all the aspects of this invention is EGFP (DNA encoding EGFP which is a F64L-S65T variant with codons optimized for expression in mammalian cells is available from Clontech, Palo Alto, plasmids containing the EGFP DNA sequence, cf. GenBank Acc. Nos. U55762, U55763). Another especially preferred variant of GFP is F64L-E222G-GFP.

Another method of recording the localisation of the PDE4 by fluorescence methods uses standard chemical means to label purified PDE4 proteins with fluorophores such as fluorescein, BODIPY or Cy dyes, or rhodamine. Labelling is performed using reactive forms of these dyes as supplied by, for example, Molecular Probes Inc. (Oregon, USA), and allowing the proteins to react under the conditions and protocols recommended by the manufacturers of these reagents. Following chemical labelling and appropriate purification the probes can be microinjected into cells by standard techniques known to the art, and their behaviour within cells observed by fluorescence techniques.

It is desirable, but not always necessary, that the identity of the probe, and hence its cellular distribution, can be followed in living cells. This allows for the progress of transient changes in distribution to be recorded. Thus a preferred aspect of the invention is a method as described, wherein the comparison between the effect of the reagent and the effect of the compound is based on a time series of measurements.

When an assay as been set up, one aspect of such assay is precise knowledge on when (in a time series) the effect is seen. Then, in order to optimise the screening and to minimise the data-output, the invention relates to a method as described, wherein the comparison between the effect of the reagent and the effect of the compound is based on an end-point measurement.

In general, a probe, i.e. a “GeneX”-GFP fusion or a GFP-“GeneX” fusion, is constructed using PCR with “GeneX”-specific primers followed by a cloning step to fuse “GeneX” in frame with GFP. The fusion may contain a short vector derived sequence between “GeneX” and GFP (e.g. part of a multiple cloning site region in the plasmid) resulting in a peptide linker between “GeneX” and GFP in the resulting fusion protein.

Some of the steps involved in the development of a probe include the following: Identify the sequence of the gene. This is most readily done by searching a depository of genetic information, e.g. the GenBank Sequence Database, which is widely available and routinely used by molecular biologists. In the specific examples below the GenBank Accession number of the gene in question is provided.

Design the gene-specific primers. Inspection of the sequence of the gene allows design of gene-specific primers to be used in a PCR reaction. Typically, the top-strand primer encompasses the ATG start codon of the gene and the following ca. 20 nucleotides, while the bottom-strand primer encompasses the stop codon and the ca. 20 preceding nucleotides, if the gene is to be fused behind GFP, i.e. a GFP-“GeneX” fusion. If the gene is to be fused in front of GFP, i.e. a “GeneX”-GFP fusion, a stop codon must be avoided. Optionally, the full-length sequence of GeneX may not be used in the fusion, but merely the part that localizes and redistributes like GeneX in response to a signal. In addition to gene-specific sequences, the primers contain at least one recognition sequence for a restriction enzyme, to allow subsequent cloning of the PCR product. The sites are chosen so that they are unique in the PCR product and compatible with sites in the cloning vector. Furthermore, it may be necessary to include an exact number of nucleotides between the restriction enzyme site and the gene-specific sequence in order to establish the correct reading frame of the fusion gene and/or a translation initiation consensus sequence. Lastly, the primers always contain a few nucleotides in front of the restriction enzyme site to allow efficient digestion with the enzyme.

Identify a source of the gene to be amplified. In order for a PCR reaction to produce a product with gene-specific primers, the gene-sequence must initially be present in the reaction, e.g. in the form of cDNA. Information in GenBank or the scientific literature will usually indicate in which tissue(s) the gene is expressed, and cDNA libraries from a great variety of tissues or cell types from various species are commercially available, e.g. from Clontech (Palo Alto), Stratagene (La Jolla) and Invitrogen (San Diego). Many genes are also available in cloned form from The American Type Tissue Collection (Virginia).

Optimise the PCR reaction. Several factors are known to influence the efficiency and specificity of a PCR reaction, including the annealing temperature of the primers, the concentration of ions, notably Mg²⁺ and K⁺, present in the reaction, as well as pH of the reaction. If the result of a PCR reaction is deemed unsatisfactory, it might be because the parameters mentioned above are not optimal. Various annealing temperatures should be tested, e.g. in a PCR machine with a built-in temperature gradient, available from e.g. Stratagene (La Jolla), and/or various buffer compositions should be tried, e.g. the OptiPrime buffer system from Stratagene (La Jolla).

Clone the PCR product. The vector into which the amplified gene product will be cloned and fused with GFP will already have been taken into consideration when the primers were designed. When choosing a vector, one should at least consider in which cell types the probe subsequently will be expressed, so that the promoter controlling expression of the probe is compatible with the cells. Most expression vectors also contain one or more selective markers, e.g. conferring resistance to a drug, which is a useful feature when one wants to make stable transfectants. The selective marker should also be compatible with the cells to be used.

The actual cloning of the PCR product should present no difficulty as it typically will be a one-step cloning of a fragment digested with two different restriction enzymes into a vector digested with the same two enzymes. If the cloning proves to be problematic, it may be because the restriction enzymes did not work well with the PCR fragment. In this case one could add longer extensions to the end of the primers to overcome a possible difficulty of digestion close to a fragment end, or one could introduce an intermediate cloning step not based on restriction enzyme digestion. Several companies offer systems for this approach, e.g. Invitrogen (San Diego) and Clontech (Palo Alto).

Once the gene has been cloned and, in the process, fused with the GFP gene, the resulting product, usually a plasmid, should be carefully checked to make sure it is as expected. The most exact test would be to obtain the nucleotide sequence of the fusion-gene.

Once a DNA construct for a probe has been generated, its functionality and usefulness may be evaluated by transfecting it into cells capable of expressing the probe.

Some of the advantages of using live cells in the design and operation of assays to screen for therapeutic drugs include the inherent ability of the assay to determine the availability of any compound to targets in the cell interior, and also an inherent assessment of the possible toxicity of a test compound or its cellular metabolites over the period of the assay, for example the PDE4A4 spot assay can involve up to 24 hours exposure of cells to test compounds before the readout, or measurement, is made, during which time any immediate toxic effects of test compounds or their cellular metabolites on cells can be observed.

Numerous cell systems for transfection exist. A few examples are Xenopus oocytes or insect cells, such as the sf9 cell line, or mammalian cells isolated directly from tissues or organs taken from healthy or diseased animals (primary cells), or transformed mammalian cells capable of indefinite replication under cell culture conditions (cell lines). However, it is preferred that the cells used are mammalian cells. This is due to the complex biochemical interactions specific for each cell type.

The fluorescence of the cell is inspected soon after, typically the next day. At this point, two features of cellular fluorescence are noted: the transfection intensity and the sub-cellular localisation.

The intensity should usually be at least as strong as that of unfused GFP in the cells. If it is not, the sequence or quality of the probe-DNA might be faulty, and should be carefully checked. Other causes of poor expression can often be corrected by linearising the plasmid DNA prior to transfection, or by increasing the concentration of DNA used for a transfection process, or by choosing a different transfection agent or method, of which many are known to those skilled in the art.

The sub-cellular localisation is an indication of whether the probe is likely to perform well. If it localises as expected for the gene in question, e.g. form spots upon treatment with Rolipram, it can immediately go on to a functional test. If the probe is not localised soon after the transfection procedure, it may be because of overexpression at this point in time, as the cell typically will have taken up very many copies of the plasmid, and localisation will occur in time, e.g. within a few weeks, as plasmid copy number and expression level decreases. If localisation does not occur after prolonged time, it may be because the fusion to GFP has destroyed a localisation function, e.g. masked a protein sequence essential for interaction with its normal cellular anchor-protein. In this case the opposite fusion might work, e.g. if GeneX-GFP does not work, GFP-GeneX might, as two different parts of GeneX will be affected by the proximity to GFP. If this does not work, the proximity of GFP at either end might be a problem, and it could be attempted to increase the distance by incorporating a longer linker between GeneX and GFP in the DNA construct. Lack of proper localisation may also be due to a lack of suitable anchorage or scaffold sites within the cell, which can often be corrected by co-transfection of genes coding for the the protein component or components responsible for providing the appropriate anchorage or scaffold sites.

If there is no prior knowledge of localisation, and no specific localisation is observed, it may be because the probe should not be localised at this point, because such is the nature of the protein fused to GFP. It should then be subjected to a functional test.

In a functional test, the cells expressing the probe are treated with at least one Rolipram-like reference compound. If a redistribution is observed and if prior knowledge suggests that it should translocate from location X to location Y, then the probe has passed the first critical test. In this case it can go on to further characterisation and quantification of the response.

If it does not perform as expected, it may be because the cell lacks at least one component of the signalling pathway, e.g. a cell surface receptor, or the anchoring site is absent or saturated, or there is species incompatibility, e.g. if the probe is modelled on sequence information of a human gene product, and the cell is of hamster origin. In both instances one should identify other cell types for the testing process where these potential problems would not apply.

In the present invention preferred fusion probes are listed in Table 3. Most preferred probes are PDE4 probes of human origin, fusion probes like HSPDE4A1-EGFP, HSPDE4A4-EGFP, HSPDE4A4-H506N-EGFP, and HSPDE4A4-ΔLR2-EGFP. The construction and testing of the probes used in the scientific findings of the present invention are described in examples 1 and 2. Terminology used here for PDE4 proteins follows the recommendations of the nomenclature committee (Beavo et al., 1994). The recommendations of the committee are that the first two letters should indicate the source species (HS, Homo sapiens; RN, Rattus norvegicus), that PDE be used to designate cyclic nucleotide phophodiesterase, that an arabic numeral indicate the superfamily (4, in this case), that a single letter indicate the gene family (A, B, C, D for PDE4), that another arabic numeral indicate the splice variant, and finally a single letter be used for the report describing the enzyme in question (generally omitted after validation and full description of the isoform).

The behaviour and use of probes based on PDE4A may also be applied to gene families B, C and D of PDE4 through exchange of their catalytic domains into the structure of PDE4As. Since there exist identifiably homologous regions within the catalytic domains of the four gene families, hybrid molecules can conveniently be constructed as follows: A conserved amino acid is chosen within a stretch of the PDE4A protein's primary sequence, within the catalytic domain, that is homologous, or preferably identical, to the corresponding region in an enzyme from a different gene family of PDE4: one such region of amino acid identity is found for example between amino acids 457 and 467 (numbering from HSPDE4A4). Standard molecular biology techniques are then used to remove all codons for amino acids in the PDE4A gene sequence that are C-terminal to that position, and they are replaced with the corresponding coding sequence from a different PDE4 gene (say that of PDE4B). The PDE4 hybrid sequence is then fused to a sequence coding for a label/marker such as EGFP, such that the gene product will have EGFP attached to the C-terminal of the enzyme.

These hybrid probes, when expressed in cells, bind Rolipram and other PDE4-specific inhibitors with affinities reflecting the particular properties of the substitute catalytic site (of PDE4B in the example above), but exhibit the redistribution behaviour of the chosen PDE4A. Measurement of the redistribution behaviour reflects the binding properties specific to the imported catalytic site.

It is published that the association of certain partner proteins with certain PDE4s will affect the affinity of the enzymes for Rolipram. It is presently contemplated that specific compounds binding to the enzyme (e.g. Rolipram) will alter the mobility of the enzyme (that is the binding to an anchorage or docking partner) altering the freedom of the enzyme to move in 3 dimensional space.

One major aspect of this invention is based on the finding that incubation of cells with Rolipram causes redistribution of PDE4A probes. This redistribution is not a consequence of an increase in cAMP brought about by inhibition of PDE4 activity, since the redistribution is not simply mimicked by treatment with IBMX, or Forskolin±IBMX (example 10).

Rather, it seems that binding of Rolipram and certain other PDE4 inhibitors induces conformational changes in PDE4A leading to a change in the affinity of the PDE4A probes for their docking or anchorage partners, which results in their subsequent redistribution in the cell.

It is further speculated, from the known properties of Rolipram and the other compounds that mimic the effects of Rolipram on PDE4A redistribution, that it is the binding of these compounds to what is commonly referred to as the “high affinity Rolipram binding site” (HARBS) or “Sr”, or HPDE4 form, (Souness and Rao, 1997) that specifically triggers the changes that lead to redistribution of the PDE4A probes. Specific PDE4 inhibitors such as RP73401 and SB207499 do not recognise HARBS, but apparently recognise an alternative site within the catalytic domain and inhibit the cAMP hydrolysing ability of the catalytic site (Sc) in a somewhat different way; Rolipram also binds in this second way, and in this case it is said to bind to a “low affinity site”, or to the LPDE4 form (Souness and Rao, 1997). Since RP73401 does not cause redistribution of PDE4A probes, it is predicted that SB207499 will also fail to cause redistribution of these probes, and further, that like RP73401, SB207499 will compete against and reverse the redistribution caused by Rolipram and other compounds that bind to the Sr. It is presently unknown whether HARBS and the low affinity site (or sites), are truly distinct and separate positions within the catalytic cleft of PDE4 enzymes, or whether they represent different conformational states of the same site. The role of binding to the Sc or the Sr, in terms of elevation of cAMP and inhibition of cell responses, is not yet fully understood. The importance of the Sr in influencing the pharmacological profile of PDE4 inhibitors may have implications in predicting not only efficacy but also the side-effects of these drugs, such as nausea, emesis, excess gastric acid secretion and headache, which have hampered the clinical development of PDE4 inhibitors to date. (Souness & Rao, 1997)

The majority of the experiments in the present application are based on the effects of Rolipram. Thus, it is preferred that the method of the invention is carried out using Rolipram as a reference compound. However, in another embodiment of the present invention, a Rolipram-like reference compound is used. A Rolipram-like reference compound is a compound, sharing the properties of Rolipram with regards to their ability to cause redistribution of the PDE probe being used and with regards to the ability to inhibit the catalytic activity of the PDE4.

It is an important aspect of the present invention that a change in localisation of the PDE4 is detected as a consequence of the treatment with the Rolipram-like reference compound. Examples 1-14 illustrates the formation of spots caused by Rolipram. Thus it is preferred that the method relates to a change in localisation of the PDE4 as formation of spots. Example 15 illustrates the dispersal of spots caused by Rolipram treatment. Thus, it is another preferred embodiment that the method relates to a change in localisation of the PDE4 as dispersal of spots.

One aspect of the present invention relates to the identification of a compound that produces a distinct change in intracellular distribution of the probe, such as a test compound that will mimic the distinct change in localisation caused by the Rolipram-like reference compound, and by inference that the test compound and the Rolipram-like reference compound will share a common pharmacological profile.

This identification of compounds with an agonistic effect is preferably carried out as a method to monitor changes in intracellular distribution of PDE4s in living cells caused by a test compound, the method comprising the steps of:

O1) optionally constructing a probe allowing the location of the PDE4 to be recorded;

O2) optionally transfecting cells with the constructed probe of step (O1);

(a) recording the intracellular distribution of the PDE4;

(b) adding a Rolipram-like reference compound to the cells in (a) or to similar cells, the Rolipram-like reference compound being able to bind to the catalytic cleft of the PDE4;

(b1) adding the test compound to the cells in (a) or similar cells;

(b2) recording the intracellular distribution of the PDE4 in the cells in step (b1);

(c) recording the intracellular distribution of the PDE4 in the cells in step (b);

(d) determining the effect on the intracellular distribution of the PDE4 of the Rolipram-like reference compound by comparing the intracellular distribution recorded in step (a) with the intracellular distribution recorded in step (c);

(d1) determining the effect of the test compound by comparing the intracellular distribution recorded in step (b2) with the intracellular distribution recorded in step (a);

the pharmacology of the test compound being established by comparing the determined effect in step (d1) with the determined effect in step (d) a substantial copy of the effect determined in step (d), in step (d1), being indicative of an agonistic effect of the test compound to the Rolipram-like reference compound in regards to the change in intracellular distribution of the PDE4.

Agonists induce the formation of very bright spots, often a single pair, in cells expressing the HSPDE4A4-EGFP probe. Certain agonists (RS25344, but not Rolipram) will induce formation of the same kind of spots in cells expressing the H506N mutant of this probe, indicating their ability to “bridge” or compensate for the mutation which removes Rolipram agonism. In all cases, formation of bright spots requires protein synthesis and accumulation of probe: two hour incubation with Rolipram, or other agonists, is sufficient to determine that spot formation is under way, but the spots become larger and brighter, and therefore easier to measure, after a total of about 6 hours incubation with the test compound. Between 6 to 24 hours, spot numbers do not increase greatly, although their size and brightness does continue to grow. Incubation of cells with test compounds for a period of 16 hours before fixation proves to be a convenient and reliable method to screen batches of compounds, allowing many plates of cells to be treated in the evening, incubated overnight, and fixed, stained and analysed the following morning. With the HSPDE4A1-EGFP probe, agonists induce dispersal of the bright spots that normally lie in the perinuclear area of cytoplasm. Dispersal of spots is easily measurable after 60 to 90 minutes, and therefore is a faster process than spot formation with HSPDE4A4-EGFP.

Agonists found through use of either probe may be expected to be specific PDE4 inhibitors, and a suitable secondary screen for PDE4 specific inhibition is desirable to confirm this property.

Another aspect of the present invention relates to the identification of a test compound that will prevent and reverse the distinct change in localisation produced by action of the Rolipram-like reference compound e.g. by displacing the Rolipram-like reference compound.

This identification of test compounds with an antagonistic effect is preferably carried out as a method to monitor changes in intracellular distribution of PDE4s in living cells, the method comprising the steps of:

O1) optionally constructing a probe allowing the location of the PDE4 to be recorded;

O2) optionally transfecting cells with the constructed probe of step (O1);

(a) recording the intracellular distribution of the PDE4;

(b) adding a Rolipram-like reference compound to the cells in (a) or to similar cells, the Rolipram-like reference compound being able to bind to the catalytic cleft of the PDE4;

(b1) adding a test compound to the cells with the Rolipram-like reference compound in step (b) or similar cells;

(b2) recording the intracellular distribution of the PDE4 in the cells in step (b1);

(c) recording the intracellular distribution of the PDE4 in the cells in step (b);

(d) determining the effect on the intracellular distribution of the PDE4 of the Rolipram-like reference compound by comparing the intracellular distribution recorded in step (a) with the intracellular distribution recorded in step (c).;

(d1) determining the effect of the test compound by comparing the intracellular distribution recorded in step (b2) with the intracellular distribution recorded in step (a);

the pharmacology of the test compound being established comparing the determined effect in step (d1) with the determined effect in step (d) a reversal, in step (d1), of the effect determined in step (d) to an effect substantially identical to the effect determined in step (a) being indicative of an antagonistic effect of the test compound on the Rolipram-like reference compound in regards to the change in intracellular distribution.

The pharmacology of the test compound can also be established by comparing the determined effect in step (d1) with the determined effect in step (d) an increased effect, in step (d1), of the effect determined in step (d), comparable to an effect in step (d) obtained with a higher doses of the Rolipram-like reference compound, being indicative of the augmenting effect of the test compound on the Rolipram-like reference compound in regards to the change in intracellular distribution.

Antagonists induce the dispersal of the very bright spots formed by the Rolipram-like reference compound in cells expressing the HSPDE4A4-EGFP probe. Dispersal of bright spots does not require protein synthesis, and is generally easily measurable after 30 to 60 minutes. Some compounds at higher concentrations, such as RP73401, can disperse spots very rapidly; spots formed by 2 μM Rolipram over 16 hours will disperse within 10 minutes with 1 μM RP73401. A screen for antagonists may involve incubating cells with Rolipram-like reference compound (say 3 μM Rolipram, or 0.5 μM RS25344) for a period of 16 hours, then adding the test compound and incubating further for a period of 60 minutes before being fixed, stained and analysed.

With the HSPDE4A1-EGFP probe, antagonists reverse the dispersal of perinuclear bright spots that normally results from treatment with Rolipram-like reference agonists such as Rolipram. Compounds may be added simultaneously with Rolipram-like reference compounds, or at some time later (such as after 60 to 90 minutes incubation with the Rolipram-like reference compound). Reappearance of spots is easily measurable after 240 minutes.

As detailed in examples 10, 11 and 12, certain treatments are known to disperse PDE4A4 Rolipram spots in CHO cells. These include [Forskolin+IBMX] (example10) and [PMA±ionomycin] (example 11). Appropriate counterscreens will help to identify compounds that redistribute PDE4s through dislocation: dislocator compounds will not bind to the catalytic cleft, so will not inhibit catalytic activity of PDE4s, will not induce increase in cAMP/activation of PKA in cells (as forskolin+IBMX does), will not mimic the effects of PMA±ionomycin, i.e. directly stimulate PKC isoforms, perhaps through prolonged increase of intracellular Ca²⁺ or through increase in levels of diacyl glycerol. Since the antagonist assay using the PDE4A4 probe detects compounds by their ability to disperse spots, this assay is also useful in detecting compounds that dislocate PDEs, or their anchor protein(s), from their preferred cellular location. If a compound is found to disperse spots in the PDE4A4 antagonist assay, AND causes spots to reform, or persist, in the 4A1 antagonist assay, that compound is most likely to be a PDE4 specific inhibitor with little affinity for the HARB site, and should have properties similar to RP73401 (and, as predicted, to Ariflo®, or SB207499). If a compound found in the PDE4A4 antagonist assay fails to cause spots to reform or persist in the 4A1 antagonist assay, and does not screen as positive in the suggested counterscreens, that compound is likely to be a dislocator of PDE4A4, or it's anchor protein(s). By extension, a compound with activity in the 4A1 agonist assay, but no activity in the PDE4A4 agonist assay, and which does not screen positive in a PDE4 inhibition assay, is likely to be a dislocator of PDE4A1 or it's anchor protein(s).

Test compounds identified by the method of the present invention include specific inhibitors of PDE4 enzymes, which can be categorised from their effect on the distribution of PDE4A probes as being either Rolipram-like, or non Rolipram-like inhibitors. It is speculated that the crucial property all Rolipram-like inhibitors share, is the ability to bind to the high affinity Rolipram binding site of PDE4 enzymes, and/or the ability to trigger a conformational change in PDE4A enzymes from an interaction within the catalytic cleft. Test compounds identified by the method of the present invention are also predicted to include dislocator compounds, which either disrupt/enhance the association of PDE4 isoenzymes with particular anchor proteins or to disrupt/enhance machinery responsible for the trafficking of PDE4 proteins between different locations within the cell. In so doing, compounds are identified whose usage would be in disrupting or relocalising the placement of a PDE4 isoenzyme in its established place in the cell so as to enhance compartmentalised cAMP function. Through this novel approach and the derivation of appropriate assays an entirely new way of generating PDE4 isoform-selective therapeutics is envisaged.

It is preferred that the test compound identified, e.g. as an agonist or an antagonist, is a single substance composed of one or more chemical elements. An example of such a test compound is a peptide.

The term “compound” is intended to indicate any sample which has a biological function or exerts a biological effect in a cellular system. The sample may be a sample of a biological material such as a sample of a body fluid including blood, plasma, saliva, milk, urine, or a microbial or plant extract, an environmental sample containing pollutants including heavy metals or toxins, or it may be a sample containing a compound or mixture of compounds prepared by organic synthesis or genetic techniques.

In a further aspect of the invention it is preferred that the test compound binds to the catalytic cleft of the PDE4. The catalytic cleft of the PDE4 is the cleft within the protein macromolecule to which a substrate for the enzyme is introduced, and where conditions for a specific chemical (or physical) reaction involving the substrate are thermodynamically optimised for the reaction to run in a particular direction. For the PDE4s it lies within the region recognised as the conserved catalytic domain, which (using amino acid numbering from HSPDE4A4) has been determined from a combination of truncation and deletion experiments as comprising some 315 to 348 amino acids located between residues 332/365 and 680 (Houslay, Sullivan and Bolger, 1998). Mutations and deletions within this consensus region are likely to ablate or decrease cAMP binding and hydrolytic abilities of PDE4 enzymes. The terms catalytic site and active site have a similar meaning in this regard.

The affinity with which a test or Rolipram-like reference compound binds to the catalytic cleft may be determined through use of standard radioligand binding assays, wherein the test compound is radiolabelled and incubated with a more or less purified preparation of the target PDE4 enzyme. Such an enzyme preparation may be obtained from cells transfected with and expressing the PDE4 enzyme chosen. Typical systems used in such a procedure may be found described in Saldou et al. (1998). Alternatively displacement of tritiated Rolipram from brain-derived microsomal preparations can be used to determine the affinity of a test compound for the so-called high-affinity Rolipram binding site of PDE4 enzymes.

Binding affinity, effects on PDE4A probe redistribution and inhibitory effects on catalytic activity are not necessarily correlated. In certain aspects of the invention is preferred that test compounds found through such use of the invention also inhibit the catalytic activity of PDE4s. Effects of a test compound on the catalytic activity of a PDE4 can easily be determined by standard competitive binding experiments between PDE inhibitors and cAMP on enzyme activity for which known amounts of cAMP substrate and fixed amounts of enzyme are incubated together with various amounts of inhibitor substance for fixed periods of time, after which the reaction is stopped and the residual amount of unhydrolysed cAMP is measured. This may be done for any test sample by use of a scintillation proximity based assay (SPA) designed to measure the competition between cAMP in the test sample and a known amount of radiolabelled cAMP for binding to a cAMP-specific antibody attached to scintillant beads (Hancock et al., 1995). The assay is read in a scintillation counter where the counts per sample are inversely related to the amount of cAMP present in the test sample. SPA kits for measurement of cAMP are available from Amersham Pharmacia Biotech (Amersham, UK).

Yet another class of compounds that can be detected by the method of the invention is described inter alia in example 13. These compounds inhibit the reappearance of spots in cells expressing the PDE4A4 probe, where the spot reappearance process is triggered by various imposed conditions, and is specific to cells that have formed spots under the influence of a Rolipram-like PDE4 inhibitor (an agonist compound selected from Rolipram, Ro 20-1724, RS25344 etc.), and where the cells have then been cleared of spots by removal of the agonist compound. Thalidomide is an example of a compound that is a spot reappearance inhibitor, as described in example 13.

Spot reappearance inhibitors may be compounds that inhibit cellular stress responses. The identification of test compounds as spot reappearance inhibitors is preferably carried out as a method comprising the steps of:

(1) treating PDE4A4 expressing cells with a reference agonist compound for a period to induce spots (e.g. 7-24 hours);

(2) checking that spots have formed;

(3) washing away the Rolipram-like reference compound and leaving cells in incubator for spots to completely disappear (about 150 minutes);

(4) checking that all spots have disappeared;

(5) adding the test compounds, keeping some wells as negative controls;

(6) exposing all cells to 100 mM salt and 4° C. for 4 hours, or leaving the cells at ambient conditions for 4 hours to allow cooling to 22° C., alkalinisation of medium (pH=6.5 shifting to pH=8.2) and partial evaporation (about 20% decrease in volume);

(7) determining the degree of spot reappearance compared to control wells that have not been treated with any test compound.

As will be evident to the person skilled in the art, compounds capable of inhibition of the function of PDE4 are capable of preventing/decreasing inflammation and/or depression. The present invention provides at least two novel approaches to identifying such compounds. All approaches are based on the initial discovery that Rolipram, by binding to the catalytic cleft of the PDE4, induces a change in the cellular distribution of the PDE4.

One method is a method to determine if a compound is a dislocator of PDE4, comprising the steps of:

testing if the compound removes PDE4-spots, where PDE4-spots may optionally be induced by a Rolipram-like reference compound and

testing if the compound inhibits the catalytic activity of the PDE4; the compound being a disclocator of PDE4, if the compound removes PDE4-spots and if the compound does not inhibit the catalytic activity of PDE4.

PDE4 dislocators will remove the PDE4 away from the native location in the cell and thereby increase the concentration of cAMP in said native location (‘compartment’) in the cell. Such increased concentration of cAMP is also seen upon inhibition of the catalytic activity of the PDE4, however, by dislocating the PDE4, and thereby not acting directly on the catalytic well conserved site, the compound will act e.g. at the binding domain of the PDE4, thereby providing isoform-specific ‘inhibitors’ of PDE4.

One aspect of the present invention thus relates to a PDE4 dislocator obtainable by the method described. Such PDE4 dislocator is preferably included into a pharmaceutical composition comprising a compound, the compound being a dislocator of PDE4, and the pharmaceutical composition having a market authorisation, the market authorisation being based on an application for market authorisation comprising data showing removal of PDE4-spots, optionally induced by a Rolipram-like reference compound, by the compound and lack of inhibition of the catalytic activity of PDE4 by the compound.

An example of the market authorisation is described in 65/65/EEC. The data required specified in Article 4,8. of said directive.

Preferred dislocators of PDE4 are dislocators of PDE4A isoforms, such as the PDE4A1 isoform and/or the PDE4A4 isoform.

PDE4A1 dislocators are identified by a method comprising the steps of:

testing if the compound removes PDE4A1-spots, and

testing if the compound inhibits the catalytic activity of the PDE4A1;

the compound being a disclocator of PDE4A1, if the compound removes PDE4A1-spots and if the compound does not inhibit the catalytic activity of PDE4A1.

PDE4A1 dislocators obtainable by the method described are preferably included in a pharmaceutical composition wherein the indication on the market authorisation is diseases in the central nervous system such as depression.

PDE4A4 dislocators are identified by a method comprising the steps of:

testing if the compound removes PDE4A4-spots induced by a Rolipram-like reference compound and

testing if the compound inhibits the catalytic activity of the PDE4A4;

the compound being a disclocator of PDE4A4, if the compound removes PDE4A4-spots and if the compound does not inhibit the catalytic activity of PDE4A4.

PDE4A4 dislocators obtainable by the method described are preferably included in a pharmaceutical composition wherein the indication on the market authorisation is inflammatory diseases. Examples of inflammatory diseases are joint inflammation, Crohn's disease, inflammatory bowel disease, respiratory diseases, chronic obstructive pulmonary disease (COPD), including asthma, chronic bronchitis, pulmonary emphysema, endotoxic shock, toxic shock syndrome, systemic lupus erythematosis, psoriasis, bone resorption diseases, reperfusion injury, cancer and HIV infection.

Another method according to the invention, is a method to determine if a compound is a low emesis PDE4 inhibitor comprising the steps of:

testing if the compound causes PDE4A4-spots induced by a Rolipram-like reference compound to dissolve,

testing if the compound induces re-appearance of PDE4A1-spots in cells exposed to a Rolipram-like reference compound, and

testing if the compound inhibits the catalytic activity of PDE4;

the compound being a low emesis PDE4 inhibitor if the compound removes spots induced by the Rolipram-like reference compound and induces re-appearance of PDE4A1 spots in cells exposed to the Rolipram-like reference compound and if the compound inhibits the catalytic activity of PDE4.

Low emesis PDE4 inhibitors will inhibit the catalytic activity of the PDE4, causing the anti-inflammatory and anti-depressant effects without causing the side effects as emesis, nausea, headaches, and excess gastric acidity.

It is preferred that the low emesis PDE4 inhibitor is included in a pharmaceutical composition comprising a compound, the compound being a low emesis PDE4 inhibitor, and the pharmaceutical composition having a market authorisation, the market authorisation being based on an application for market authorisation comprising data showing that the compound removes spots induced by the Rolipram-like reference compound, and that the compound induces re-appearance of PDE4A1 spots in cells exposed to the Rolipram-like reference compound, and that the compound inhibits the catalytic activity of PDE4.

In one aspect the indication on the market authorisation is inflammatory diseases.

Another aspect relates to the use of a Low emesis PDE4 inhibitor or a PDE4 dislocator, or a pharmaceutically acceptable salt, ester, amide or prodrug thereof, for the preparation of medicament for the treatment of inflammatory diseases including joint inflammation, Crohn's disease, and inflammatory bowel disease; respiratory diseases such as chronic obstructive pulmonary disease (COPD) including asthma, chronic bronchitis, and pulmonary emphysema; infections diseases including endotoxic shock and toxic shock syndrome; immune diseases including systemic lupus erythematosis and psoriasis; and other diseases including bone resorption diseases and reperfusion injury and conditions associated with proliferating hematopoitic cells, such as cancer and HIV infection; diseases in the central nervous system including depression.

Another important aspect of the present invention relates to the use of a Low emesis PDE4 inhibitor, or a pharmaceutically acceptable salt, ester, amide or prodrug thereof, capable of competing against and reversing the effect of the Rolipram-like reference compound on the intracellular distribution of the PDE4 and being capable of inhibiting the catalytic activity of the PDE4 for the preparation of medicament for the treatment of inflammatory diseases, e.g. rheumatoid arthritis.

Yet another aspect of the invention is a method for treating inflammatory diseases, e.g. asthma, or depression in an individual comprising administering to the individual an effective amount of a compound, or a pharmaceutically acceptable salt, ester, amide or prodrug thereof, the compound being capable of competing against and reversing or mimicking the effect of the Rolipram-like reference compound on the intracellular distribution of the PDE4 and the compound being capable of mimicking the effect of the Rolipram-like reference compound on the catalytic activity of PDE4s.

The invention further relates to the test compound identified, or identifiable, by a method according to the invention. E.g. a Low emesis PDE4 inhibitor or a PDE4 dislocator.

It is evident to the skilled person, that a PDE4 dislocator or a Low emesis PDE4 inhibitor identified by the methods described will need further testing prior to human trials. Apart from the toxicology requirements, the PDE4 dislocator is tested in functional assays for relevant action, and counter indications, at both the cellular and organismal level. Examples of such assays are the in vitro measurement of LPS-stimulated TNFα release from human peripheral blood mononucleocytes (e.g. as described by Barnette et al. 1998), and an in vivo measurement of anti-inflammatory action such as suppression of antigen-induced eosinophilia and bronchoconstriction in rat lung (e.g. as described by Hughes et al. 1996, an Asthma model) or in vivo measurement of an anti-depressant function such as the induction of brain-derived neurotrophic factor (BDNF) in rat hippocampus (a depression model; Fujimaki et al. 2000) or in vivo measurement of the amelioration of collagen II-induced arthritis in rats (a rheumatoid arthritis model; Nyman et al. 1997). Additionally, PDE4 dislocator compounds are screened for unwanted potential emetic properties in a ferret emesis test (e.g. as described by Robichaud et al. 1999).

The pharmaceutical compositions described herein may be formulated according to conventional pharmaceutical practice, see, e.g., “Remington's Pharmaceutical Sciences” and “Encyclopedia of Pharmaceutical Technology”.

It is likely that mutations in an individual within the PDE4 can be the cause of certain disease states e.g. immunological illnesses and depression. Those individuals can, in one aspect of the invention, be diagnosed for functional mutations in the the Rolipram binding site, or in the anchor binding site

by fishing the PDE4 subtype (e.g. by PCR)

fuse the PDE4 subtype to GFP

add Rolipram

measure change in distribution of the PDE4.

Another important aspect of the invention is a method for providing the basis for diagnostic methods for the early and accurate detection and/or quantitation of PDE distribution associated with joint inflammation, Crohn's disease, and inflammatory bowel disease; respiratory diseases such as chronic obstructive pulmonary disease (COPD) including asthma, chronic bronchitis, and pulmonary emhpysema; infections diseases including endotoxic shock and toxic shock syndrome; immune diseases including systemic lupus erythematosis and psoriasis; and other diseases including bone resporption diseases and reperfusion injury and conditions associates with proliferating hematopotietic cells, such as cancer and HIV infection.

### REFERENCES

Ahluwalia, G. S., et al. (1982) Biochem. Pharmacol. 31: 665

Barnette M S, Christensen S B, Essayan D M, Grous M, Prabhakar U, Rush J A, Kagey-Sobotka A, Torphy T J (1998) S B 207499 (Ariflo), a potent and selective second-generation phosphodiesterase 4 inhibitor: In vitro anti-inflammatory actions. Journal of Pharmacology & Experimental Therapeutics 284:420-426

Beavo, J. A., Conti, M., Heaslip, R. J. (1994) Multiple cyclic nucleotide phosphodiesterases. Mol. Pharmacol. 46:399-405

Conti M, Jin S L C (1999) The molecular Biology of cyclic nucleotide phosphodiesterases. Progress in Nucleic Acid Research 63:1-38

Fujimaki K, Morinobu S, Duman R S (2000) Administration of a cAMP phosphodiesterase 4 inhibitor enhances antidepressant-induction of BDNF mRNA in rat hippocampus. Neuropsychopharmacology 1:42-51

Hancock, A. A., Vodenlich, A. D., Maldonado, C., Janis, R. (1995) a2-adrenergic agonist-induced inhibition of cyclic AMP formation in transfected cell lines using a microtiter-based Scintillation Proximity Assay. J. of Receptor and Signal Transduction research 15:557-579

Hempel C M, Vincent P, Adams S R, Tsien R Y, Selverston A I (1996) Spatio-temporal dynamics of cyclic AMP signals in an intact neural circuit. Nature 384:166-169

Houslay M D (1998) Adaptation in cyclic AMP signalling processes: a central role for cyclic AMP phosphodiesterases. Semin Cell Dev Biol 9:161-7

Houslay M D, Milligan G (1997) Tailoring cAMP signalling responses through isoform multiplicity. Trends in Biochemical Sciences 22:217-224

Houslay M D, Sullivan M, Bolger G B (1998) The multi-enzyme PDE4 cyclic AMP specific phosphodiesterase family: intracellular targeting, regulation and selective inhibition by compounds exerting anti-inflammatory and anti-depressant actions. Advances in Pharmacology 44:225-342

Hughes B, Howat D, Lisle H, Holbrook M, James T, Gozzard N, Blease K, Hughes P, Kingaby R, Warrellow G, Alexander R, Head J, Boyd E, Eaton M, Perry M, Wales M, Smith B, Owens R, Catterall C, Lumb S, Russell A, Allen R, Merriman M, Bloxham D, Higgs G (1996)The inhibition of antigen-induced eosinophilia and bronchoconstriction by CDP840, a novel stereo-selective inhibitor of phosphodiesterase type 4. Br. J. Pharmacol. 18:1183-91

Hughes, B., Owens, R., Perry, M., Warrellow, G., Allen, R. (1997) Drug Discovery Today 2:89-101

Jacobitz, S., Ryan, M. D., McLaughlin, M. M., Livi, G. P., DeWolf, W. E. Jr., Torphy, T. J. (1997) Mol. Pharmacol. 51: 999-1006

Manganiello V C, Murata T, Taira M, Belfrage P, Degerman E (1995) Diversity in cyclic nucleotide phosphodiesterase isoenzyme families. Archives of Biochemistry and Biophysics 322:1-13

Nyman U, Mussener A, Larsson E, Lorentzen J, Klareskog L (1997) Amelioration of collagen II-induced arthritis in rats by the type IV phosphodiesterase inhibitor Rolipram. Clin Exp Immunol 108:15-9

Pooley L, Shakur Y, Rena G, Houslay M D (1997) Intracellular localisation of the PDE4A cAMP-specific phosphodiesterase splice variant RD1 (RNPDE4A1A) in transfected human thyroid carcinoma FTC cell lines. Biochemical Journal 271:177-185

Robichaud, A. Savoie, C., Stamatiou, P. B., Tattersall, F. D., Chan, C. C. (1999) PDE4 inhibitors induce emesis in ferrets via a noradrenergic pathway. Neuropharmacology 38:289-297

Rubin, L. L. et al. (1991) J. Cell Biol. 115: 1725

Saldou, N., Obernolte, R., Huber, A., Baecker, P. A., Wilhelm, R., Alvarez, R., Li, B., Xia, L., Callan, O., Su, C., Jarnagin, K., Shelton, E. R. (1998) Cell. Signal. 10: 427-440

Schneider, H. H., et al. (1993) Eur. J. Pharmacol. 127:105-115

Shakur Y, Wilson M, Pooley L, Lobban M, Griffiths S L, Campbell A M, Beattie J, Daly C, Houslay M D (1995) Identification and characterization of the type-IVA cyclic AMP-specific phosphodiesterase RD1 as a membrane-bound protein expressed in cerebellum. Biochemical Journal 306:801-809

Souness J E, Rao S (1997) Proposal for pharmacologically distinct conformers of PDE4. Cellular Signalling 9:227-236.

### EXAMPLES

### Example 1

Cloning and Construction of the GFP-Labelled PDE Probes

Here is described the cloning and construction of specific PDE4A variants fused to a GFP. Currently at least 5 PDE4A splice variants are known. These all share C-terminal sequences but differ in their N-termini, where targeting sequences of PDE4As are believed to be located. To best preserve the normal distribution of PDE4As, the fusions are made between the C-terminus of the PDE4A species and the N-terminal of the GFP.

To construct the HSPDE4A1-EGFP fusion, the ca. 1.95 kb coding region of HSPDE4A1 (GenBank Acc.no. U97584) is amplified using PCR and primers 4A1-top and 4A-bottom described below. The top primer includes specific HSPDE4A1 sequences following the ATG, a Kozak sequence, and a Hind3 cloning site. The bottom primer includes the common PDE4A C-terminal sequence minus the stop codon, a BamH1 cloning site, and two extra nucleotides to preserve the reading frame when inserted into in pEGFP. The PCR product is digested with restriction enzymes hind3 and BamH1, and cloned into pEGFP (Clontech, Palo Alto; GenBank Accession number U55762) cut with Hind3 and BamH1. This produces a HSPDE4A1-EGFP fusion under the control of the CMV promoter. The resulting plasmid is referred to as PS461 and is deposited under the Budapest Treaty with Deutsche Sammlung von Mikroorganismen und Zellkulturen GmbH (DSMZ) on Apr. 17, 2000 with DSM 13449.

4A1-top:

(SEQ ID NO: 9)

5′-GTAAGCTTAAGATGCCCTTGGTGGATTTCTTC-3′,
specific for PDE4A1, 4A-bottom:

(SEQ ID NO: 10)

5′-GTGGATCCCAGGTAGGGTCTCCACCTGA-3′

To construct the HSPDE4A4-EGFP fusion, the common ca. 1.9 kb C-terminal part of HSPDE4A4 (GenBank Acc.no. L20965) is amplified using PCR with primers 4A-Ct-top and 4A -bottom described below. The sequence of the top primer contains a silent mutation which introduces a Dra1 site exactly at the beginning of the shared 4A region. The bottom primer includes the common C-terminal sequence minus the stop codon, a BamH1 cloning site, and two extra nucleotides to preserve the reading frame when cloned into pEGFP. The unique ca. 0.8 kb N-terminal part of HSPDE4A4 is amplified using PCR in the presence of 5% DMSO with primers 4A4-top and 4A4N-bottom described below. The top primer includes specific HSPDE4A4 sequences following the ATG, a Kozak sequence, and a Hind3 cloning site. The bottom primer spans the junction of the unique 4A4 N-terminal part and the common 4A C-terminal part, and it contains a silent mutation which introduces a Dra1 site exactly at the beginning of the shared 4A region. The PCR products are digested with the relevant restriction enzymes (Hind3 and Dra1 for the unique N-terminal part and Dra1 and BamH1 for the common C-terminal part), and ligated together into pEGFP (Clontech, Palo Alto; GenBank Accession number U55762) digested with Hind3 and BamH1. This produces a HSPDE4A4-EGFP fusion under the control of a CMV promoter. The resulting plasmid is referred to as PS462 and is deposited under the Budapest Treaty with Deutsche Sammlung von Mikroorganismen und Zellkulturen GmbH (DSMZ) on Apr. 17, 2000 with DSM 13450.

4A-Ct-top:

(SEQ ID NO: 11)

5′-GTTTAAAAGGATGTTGAACCGTGAGCTC-3′
4A-bottom:

(SEQ ID NO: 12)

5′-GTGGATCCCAGGTAGGGTCTCCACCTGA-3′
4A4-top:

(SEQ ID NO: 13)

5′-GTAAGCTTGCGCCATGGAACCCCCGACC-3′
4A4N-bottom:

(SEQ ID NO: 14)

5′-GGTTTTAAACTTGTGCGAGGCCATCTCGCTGAC-3′

Catalytically inactive PDE4 fusions that redistribute normally within the cell, can be constructed by introducing specific point mutations in the catalytic domain. Use of such fusions may be advantageous if a cell is sensitive to some overexpression of a catalytically active PDE4. Many mutations are known in PDE4As that greatly reduces catalytic activity, e.g. H506N in HSPDE4A4.

Plasmid PS535 (HSPDE4A4-H506N-EGFP) is a variant of PS462 (HSPDE4A4-EGFP) containing a substitution of His-506 to Asn in HSPDE4A4. This substitution is introduced using the PCR-based Quickchange mutagenesis kit (Stratagene, La Jolla). The PCR reaction leading to this substitution utilises plasmid PS462 as template and the complementary primers 4AH-N-forward and 4AH-N-reverse shown below. In addition to the substitution, these two primers contain a silent mutation that removes an Xho I restriction site, a feature that can be used to quickly distinguish mutants from the original template. Plasmid PS535 is deposited under the Budapest Treaty with Deutsche Sammlung von Mikroorganismen und Zellkulturen GmbH (DSMZ) on Apr. 17, 2000 with DSM 13451.

Plasmid PS533 (HSPDE4A4-ΔLR2-EGFP) is a deletion mutant of plasmid PS462 (HSPDE4A4-EGFP). In plasmid PS533, 8 amino acid residues comprising the region Ala-313 to Gln-320 in the linker region 2 (LR2) of HSPDE4A4 is deleted using the PCR-based Quickchange mutagenesis kit (Stratagene, La Jolla). The PCR reaction leading to this deletion uses plasmid PS462 as template and the primers 4AΔLR2-forward and 4AΔLR2-reverse shown below. In addition to the deletion, these two primers introduce an Acc65 I restriction site by a silent mutation, which can be used to quickly distinguish mutants from the original template. Plasmid PS533 is deposited under the Budapest Treaty with Deutsche Sammlung von Mikroorganismen und Zellkulturen GmbH (DSMZ) on Apr. 17, 2000 with DSM 13452.

4AH-N-forward:

(SEQ ID NO: 15)

5′-GATGAGTCGGTGCTCGAAAATCACAACCTGGCCGTGGGCTTCAAGCT
GC
4AH-N-reverse:

(SEQ ID NO: 16)

5′-GCAGCTTGAAGCCCACGGCCAGGTTGTGATTTTCGAGCACCGACTCA
TC
4AΔLR2-forward primer:

(SEQ ID NO: 17)

5′-CCCATCACCCACGATGAAGGAACGAGAAAAACAGCAACCGCCCCCGC
CCCCGGTACCACACTTACAGCCC
4AΔLR2-reverse primer:

(SEQ ID NO: 18)

5′GGGCTGTAAGTGTGGTACCGGGGGCGGGGGCGGTTGCTGTTTTTCTCG
TTCCTTCATCGTGGGTGATGGG

To construct HSPDE4A4catD-EGFP fusion, a 5′-end fragment of HSPDE4A4 and the common 3′-end catalytic region of HSPDE4D isoforms are amplified separately by PCR and ligated into pEGFP.

The 5′end of HSPDE4A4 (nucleotides 1-1023; GenBank accession number L20965) is amplified by PCR in the presence of 5% DMSO using plasmid PS462 (HSPDE4A4 in pEGFP) as template and primers 4A4-EcoRI-top and 4A4-HindIII-bottom described below. The 4A4-BamHI-top primer contains an EcoRI restriction site followed by a Kozak sequence and the ATG of HSPDE4A4. The 4A4-HindIII-bottom primer contains a HindIII site that has been introduced into the HSPDE4A4 sequence of the primer by two silent mutations (underlined in the primer sequence). These mutations preserve correct translation of the protein and allow ligation to the HSPDE4Dcat fragment. For the amplification of the common 3′-end catalytic region of HSPDE4D isoforms, HSPDE4Dcat, the HSPDE4D3 isoform (GenBank accession number L20970) is amplified from a pool of placenta and fetal brain cDNA (Clontech, Palo Alto) using the primers 9855 and 9858 described below. Primer 9855 contains a HindIII restriction site and nucleotides specific for the 5′-end of HSPDE4D3 including the ATG start codon. Primer 9858 contains an EcoRI restriction site and nucleotides specific for the 3′-end of HSPDE4D except for the stop-codon. This fragment is digested with HindIII and EcoRI and ligated into the corresponding sites in pEGFP (Clontech, Palo Alto). The resulting plasmid is termed PS449. From this plasmid, the 3′-end catalytic region of HSPDE4D isoforms (nucleotides 700-2019 of HSPDE4D3 cDNA; GenBank accession number L20970) is amplified by PCR using primers 4Dcat-HinDIII-top and 4Dcat-SacII-bottom as described below. The 4Dcat-HinDIII-top contains a HindIII restriction site that has been integrated into the HSPDE4D sequence by two silent mutations (underlined in the primer sequence). The 4Dcat-SacII-bottom primer contains the 3′-end sequence of PDE4D isoforms except for the stop-codon in order to make fusion to EGFP possible.

The HSPDE4A4 5′-end fragment is digested with EcoRI and HindIII. The HSPDE4Dcat fragment is digested with HindIII and SacII. These two fragments are ligated in a three-part ligation into pEGFP (Clontech, Palo Alto; GenBank accession number U55762) digested with EcoRI and SacII. This results in an HSPDE4A4catD-EGFP fusion under control of a CMV promoter. The plasmid is referred to as PS687.

4A4-EcoRI-top

(SEQ ID NO: 19)

5′-CCGGAATTCCGCCATGGAACCCCCGACCGTCCCCTC
4A4-HindIII-bottom

(SEQ ID NO: 20)

5′-GGCAAGCTTTTTCAACCCTGTGATTTGGGACATGGGCTGTAAGTG
4Dcat-HinDIII-top

(SEQ ID NO: 21)

5′-GGCAAGCTTATGCACAGCTCTAGTCTGACTAATTCAAGTATCCCAAG
GTTTGG
4Dcat-SacII-bottom

(SEQ ID NO: 22)

5′-GCCCCGCGGCGTGTCAGGAGAACGATCATCTATGACACAGGCTTCAG
GC
9855:

(SEQ ID NO: 27)

5′-GTAAGCTTGCGAACATGATGCACGTGAAT
9858:

(SEQ ID NO: 28)

5′-GTGAATTCCCGTGTCAGGAGAACGATCAT

Plasmid PS716 expresses a fusion between PDE4A1 and a E222G derivative of GFP, and plasmid PS717 expresses a fusion between PDE4A4 and a E222G derivative of GFP. They were made by replacing a ca. 0.8 kb BamH1-Xba1 fragment containing EGFP sequence with a similar fragment containing the E222G derivative of GFP from plasmid PS699.

PS699 was constructed as described below.

Construction of GFP plasmid combining F64L and E222G and mammalian codon usage.

Plasmid pEGFP (GenBank accession number U55762) contain a derivative of GFP in which one extra amino acid has been added at position two to provide a better translational start sequence (a Kozak sequence) and so the total number of amino acids is increased by one to 239 instead of the 238 found in wildtype GFP. Therefore the denomination of mutations in GFP in these plasmids strictly should be referred to as e.g. F65L rather than F64L. However, to avoid this source of confusion and because the GFP community has adopted the numbering system of wildtype GFP in its communications, the numbers used here conform to the commonly used naming of mutations in wildtype GFP. The relevant mutations in this respect are F64L, S65T, and E222G.

Plasmid pEGFP contains the following mutations in the chromophore: F64L and S65T. The codon usage of the GFP DNA sequence has been optimized for expression in mammalian cells. N1 and refers to the position of multiple cloning sites relative to the GFP sequence.

To construct a plasmid combining F64L and E222G, pEGFP is first subjected to PCR with primers 9859 and 9860 described below. The primers are complementary to the DNA sequence around the chromophore region and introduce a point mutation changing the threonine at position 65 to serine. In addition the primers introduce a unique Spe1 restriction site by silent mutation. The 4.7 kb PCR product is digested with Spe1, religated, and transformed into E.coli. The resulting plasmid is referred to as PS399. This plasmid contains the chromophore sequence 64-LSYG-67. Plasmid PS399 is subjected to Quick-Change mutagenesis (Stratagene) employing PCR with primers 0225 and 0226 described below. These primers are complementary to sequences near the C-terminus of the GFP and change glutamate at position 222 to glycine, and in addition they introduce an Avr2 restriction site by silent mutation. The resulting plasmid is referred to as PS699. It combines an LSYG chromophore with E222G.

9859-top:

(SEQ ID NO: 33)

5′-TGTACTAGTGACCACCCTGTCTTACGGCGTGCA-3′
9860-bottom:

(SEQ ID NO: 34)

5′-CTGACTAGTGTGGGCCAGGGCACGGGCAGC-3′
0225-bottom:

(SEQ ID NO: 35)

5′-CCCGGCGGCGGTCACGAACCCTAGGAGGACCATGTGATCGCG-3′
0226-top:

(SEQ ID NO: 36)

5′-CGCGATCACATGGTCCTCCTAGGGTTCGTGACCGCCGCCGGG-3′

TABLE 3

| | | Predicted amino |
|---|---|---|
| Name | Nucleotide sequence | acid sequence |
| HSPDE4A1-EGFP | SEQ ID NO: 1 | SEQ ID NO: 2 |
| HSPDE4A4-EGFP | SEQ ID NO: 3 | SEQ ID NO: 4 |
| HSPDE4A4-deILR2-EGFP | SEQ ID NO: 5 | SEQ ID NO: 6 |
| HSPDE4A4-H506N-EGFP | SEQ ID NO: 7 | SEQ ID NO: 8 |
| HSPDE4A4catD-EGFP | SEQ ID NO: 23 | SEQ ID NO: 24 |
| HSPDE4D3-EGFP | SEQ ID NO: 25 | SEQ ID NO: 26 |
| HSPDE4A1-E222G | SEQ ID NO: 29 | SEQ ID NO: 30 |
| HSPDE4A4-E222G | SEQ ID NO: 31 | SEQ ID NO: 32 |

### Example 2

In vivo Expression, Visualisation and Measurement of Changes Undergone by the Probes

Transfection and Cell Culture:

Chinese hamster ovary cells (CHO), are transfected with the plasmids described in Example 1 above using the transfection agent FuGENE™ 6 (Boehringer Mannheim Corp, USA) according to the method recommended by the suppliers. Stable transfectants are selected using 1 mg/ml G418 sulphate (Calbiochem) in the growth medium (HAM's F12 nutrient mix with Glutamax-1, 10% foetal bovine serum (FBS), 100 μg penicillin-streptomycin mixture ml⁻¹ (GibcoBRL, supplied by Life Technologies, Denmark). Cell are cultured at 37° C. in 100% humidity and conditions of normal atmospheric gases supplemented with 5% CO₂.

Clonal cell lines with particular properties are subcultured from mixed populations of stably transfected cells by isolating individual cells and removing them to sterile culture flasks containing fresh culture medium with 1 mg/ml G418 sulphate.

For fluorescence microscopy, cells are allowed to adhere to Lab-Tek chambered coverglasses (Nalge Nunc International, Naperville USA) for at least 24 hours and are then cultured to about 80% confluence. Cells can also be grown in plastic 96-well plates (Polyfiltronics Packard 96-View Plate or Costar Black Plate, clear bottom; both types tissue culture treated) for imaging purposes. Prior to experiments, the cells are cultured over night without G418 sulphate in HAM F-12 medium with glutamax, 100 μg penicillin-streptomycin mixture ml⁻¹ and 10% FBS. This medium has low autofluorescence enabling fluorescence microscopy of cells straight from the incubator. For certain tests requiring medium of defined composition, particularly with regard to the presence of specific cell growth factors, the HAM's culture medium is replaced prior to imaging with a buffered saline solution (KRW buffer) containing (in mM) 3.6 KCl, 140 NaCl, 2 NaHCO₃, 0.5 NaH₂PO₄, 0.5 MgSO₄, 1.5 CaCl₂, 10 Hepes, 5 glucose, pH7.4.

Confocal Imaging:

Confocal images are collected using a Zeiss LSM 410 microscope (Carl Zeiss, Jena, Germany) equipped with an argon ion laser emitting excitation light at 488 nm. In the light path are a FT510 dichroic beamsplitter and a 515 nm long-pass filter or a 510 to 525 nm bandpass emission filter. Images are typically collected with a Fluar 40X, NA: 1.3 oil immersion objective, the microscope's confocal aperture set to a value of 10 units (optimum for this lens). Typical regions of CHO cells containing HSPDE4A1-EGFP, HSPDE4A4-EGFP, HSPDE4A4-ΔLR2-EGFP and HSPDE4A4-H506N-EGFP probes are shown in FIGS. 1, 2, 3, and 4 respectively.

Time Lapse Sequences and Analysis:

Image sequences of live cells over time (time lapse) are gathered using a Zeiss Axiovert 135M fluorescence microscope fitted with a Fluar 40X, NA: 1.3 oil immersion objective and coupled to a Photometrics CH250 charged coupled device (CCD) camera (Photometrics, Tucson, Ariz. USA). The cells are illuminated with a 100 W HBO arc lamp. In the light path are a 470±20 nm excitation filter, a 510 nm dichroic mirror and a 515±15 nm emission filter for minimal image background. The cells are maintained at 37° C. with a custom built stage heater.

Time lapse response profiles are extracted from image sequences using a region of interest (ROI) defined over the same coordinates or pixels for each successive image in a sequence: pixel values are summed and averaged over the ROI in each image, and the resulting values plotted against image number to generate a time lapse response profile for that defined region of the sequence. A ROI can include many cells, a single cell, or a region within a single cell.

Automated Imaging and Analysis:

Changes in cellular distribution of a transfected probe can be imaged and quantified in an automated fashion. For this purpose cells are cultured to near 80% to 90% confluence in coverglass chambers or plastic 96-well plates, given the relevant treatment and allowed to respond. At the end of the response period, cells are fixed in 4% formaldehyde buffer (Lillies fixative buffer, pH7.0: Bie and Berntsen A/S, Denmark) for 30 minutes to 2 hours, then washed in phosphate buffered saline (PBS, Life Technologies, Denmark). Nuclear DNA is stained with 1 μM Hoechst 33258 (Molecular Probes, Eugene, Oreg., USA) in PBS for 10 minutes at 25° C., then washed twice in PBS. Automated images are collected on a Nikon Diaphot 300 (Nikon, Japan) using a Nikon Plan Fluor 20X/0.5NA objective lens. The basic microscope is fitted with a motorised specimen stage and motorised focus control (Prior Scientific, Fulbourn, Cambridge UK), excitation filter wheel (Sutter Instruments, Novato Calif. USA) and Photometrics PXL series camera with a KAF1400 CCD chip (Photometrics, Tucson, Ariz. USA), each of these items being under the control of an Macintosh 7200/90 computer (Apple Computer, Cupertino, Calif. USA). Automation of stage positioning, focus, excitation filter selection, and image acquisition is performed using macros written in-house, running under IPLab Spectrum for Macintosh (Scanalytics, Fairfax, Va. USA). Fluorescence illumination comes from a 100 W HBO lamp. Images are collected in pairs, the first using a 340/10 nm excitation filter, the second with a 475RDF40 excitation filter (Chroma, Brattleboro, Vt.). Both images are collected via the same dichroic and emission filters, which are optimised for EGFP applications (XF100 filter set, Omega Optical, Brattleboro, Vt.). While the choice of filters for imaging the nuclear stain (Hoechst 33258) is not well matched to that dye's spectral properties, resulting in lower image intensity, it greatly improves the throughput of the procedure by allowing both images to be collected using the same dichroic and emission filter. This eliminates any image registration problems and focus shifts which would result from using two different filter sets, which would require more steps in the acquisition procedure and more extensive image processing to overcome.

The necessary images are collected as follows: A holder containing four 8-well coverglass chambers, or a single 96-well plate, is loaded onto the microscope. The program is started, and the first well of cells is moved into position and manually coarse-focused by the operator. The image is fine-focused by an auto-focus routine using the 340/10 excitation. An image is captured and stored at this excitation wavelength (the nuclear image), and then a second image is captured and stored at the longer wavelength excitation (the GFP image). The stage is automatically repositioned and microscope automatically refocused to capture a second pair of images within the same well. This process is repeated a set number of times (typically 4 to 8) for the first well. The stage then advances the next well to the imaging position, and the process repeats itself until the set number of image pairs has been captured from each well of cells.

Image pairs are automatically analysed in the following way using a suite of macros running under the IPLab Spectrum software: First the nuclear image of a pair is filtered with a digital filter to simultaneously sharpen the edges of and suppress differences in intensity of the nuclei. The choice of filter, and the filter constants, were arrived at through experimentation with various data sets. The filtered image is then segmented at a predetermined intensity value, such that pixels below this threshold are very likely not within a nuclear region, and pixels above this threshold are very likely within a nuclear region. The contiguous regions above the threshold are then counted, after rejecting contiguous regions that are larger than a certain area or smaller than a certain (different) area, the areas having been previously determined to provide a sufficiently accurate distinction between nuclei and other objects that are not nuclei. The final count is the estimated number of nuclei in the field. The GFP image of each pair is then digitally filtered with a filter chosen experimentally to suppress the variation of intensity due to the typical non-localised distribution of GFP, while accentuating the intensity of any bright point-like objects relative to this background. This filtered image is then segmented at a threshold that has been experimentally determined to divide the image into pixels that are very likely to be in a spot (above the threshold) and pixels that are very likely not to be in a spot (below the threshold). The contiguous regions of pixels that are above the threshold are counted, after rejecting regions that do not have certain morphological properties which were previously determined to be characteristic of spots. The ratio of spot count to nuclear count for each pair represents an estimate of the average number of spots per cell in that image pair. All image pairs are treated in this way, and the final table of values is used to establish the cellular response to a given treatment. Data derived from automated imaging experiments are shown in FIGS. 15 to 30, and 35 to 37.

### Example 3

Redistribution of Probe HSPDE4A4-EGFP Caused by Rolipram Treatment

This example illustrates how Rolipram affects the physical properties and behaviour of the HSPDE4A4-EGFP probe as expressed in stably transfected CHO cells.

Stably transfected (non-clonal) cells are allowed to adhere to chambered coverglasses in HAM's F12 medium containing 10% FBS. Once adhered, 2 μM Rolipram (4-(3-cyclopentyloxy-4-methoxyphenyl)-2-pyrolidone; Calbiochem) is added to the medium, and cells incubated further at 37° C. with 5% CO₂+air. At certain times after addition of Rolipram cells are checked on a fluorescence microscope for changes in cellular distribution of GFP fluorescence.

These experiments show that the general cytoplasmic distribution of fluorescence in most cells gradually changes to one consisting of bright concentrations of fluorescence located at several distinct spots within the cytoplasm, some fluorescence remaining more evenly distributed within the rest of the (non nuclear) cytoplasm. A common pattern appears to be the presence of only 2 major accumulations of fluorescence diametrically separated across the nucleus of the cell (FIG. 5). Spots are stable in cells as long as Rolipram continues to be present.

Spots begin to be visible about 3 hours after addition of 2 μM Rolipram. The effect is qualitatively similar at concentrations spanning the range 100 μM to 0.5 μM Rolipram. Pre-treatment of cells with 5 μg/ml cycloheximide prevents formation of spots induced by Rolipram, indicating that protein synthesis is a necessary part of spot formation. Once spots have formed, removal of Rolipram results in their rapid dissolution, within 60 minutes at 37° C. However, replacement of Rolipram causes the bright spots to reform, also within 60 minutes. This is more rapid than is seen for de novo production of spots by Rolipram in these cells.

These experiments indicate that the spots are built around anchor proteins that require time to be synthesised and to accumulate. Rolipram treated PDE4 appears to be necessary for this accumulation to occur. Once the accumulations of anchor proteins are formed, they remain stable within the cells, for periods of at least 4 hours.

FIG. 6 shows the homogenous response to 2 μM Rolipram of a clonal population of cells derived from a single progenitor cell transfected with probe HSPDE4A4-EGFP—more than 95% of cells have produced bright spots after 6.7 hours of exposure to Rolipram. The presence of 10% FBS is not necessary for the formation of bright spots in response to Rolipram treatment (FIG. 6).

Example 4

Redistribution of Probes HSPDE4A4-ΔLR2-EGFP and HSPDE4A4-H506N-EGFP Caused by Rolipram Treatment

This example illustrates how Rolipram affects the physical properties and behaviour of the HSPDE4A4-ΔLR2-EGFP and HSPDE4A4-H506N-EGFP probes as expressed in stably transfected CHO cells. The change in behaviour of the probe(s) is easily measurable by means of fluorescent imaging and allows this method to be used in the search for compounds that have similar properties to the PDE4 inhibitor Rolipram. Comparison of the behaviours of the 4A4 variants to that of the wild-type enzyme indicates which regions of the molecule are important in effecting the Rolipram response.

Stably transfected (non-clonal) cells are allowed to adhere to chambered coverglasses in HAM's F12 medium containing 10% FBS. Once adhered, 2 μM Rolipram (Calbiochem) is added to the medium, and cells incubated further at 37° C. with 5% CO₂+air. At certain times after addition of Rolipram cells are checked on a fluorescence microscope for changes in cellular distribution of GFP fluorescence.

Cells transfected with the HSPDE4A4-ΔLR2-EGFP probe had the same initial appearance (FIG. 3) as those transfected with the HSPDE4A4-EGFP probe, and subsequently reacted to treatment with 3 μM Rolipram in a similar way to those transfected with the HSPDE4A4-EGFP probe (FIG. 7), indicating that deletion of the region of the enzyme designated LR2 did not ablate the Rolipram response. Cells transfected with the HSPDE4A4-H506N-EGFP probe had the appearance shown in FIG. 4. Subsequent treatment with 100 μM Rolipram for 23.5 hours in HAM's F12+10% FBS causes spots to appear in only about 15% of the cells (FIG. 8). This result indicates that the histidine at position 506 in the primary sequence of the protein, located in the catalytic cleft of the enzyme, is essential to the Rolipram response. Mutation of this histidine is known to leave the Rolipram binding affinity of a shortened version of a recombinant human PDE4A enzyme virtually unchanged (Jacobitz et al., 1997), although the Km of the enzyme for cAMP increases 11 fold, and hence it's activity decreases by approximately 90%. The conclusion can therefore be drawn that the binding of Rolipram within the catalytic cleft of the enzyme initiates the change in the cellular distribution and behaviour of the HSPDE4A4-EGFP and HSPDE4A4-ΔLR2-EGFP probes via a mechanism that is dependent on the presence of this critical histidine residue at position 506. The ability of the enzyme to simultaneously bind cAMP and Rolipram may also be important in the formation of spots in cells.

### Example 5

Effects on the Cellular Distribution of HSPDE4A4-EGFP of Treatments with Several Compounds Known either to Inhibit PDE4 Enzymes Directly or Processes that are Known to be Inhibited by Rolipram

This example illustrates how various compounds with either general or specific inhibitory activity against PDEs, and/or with proven anti-inflammatory or anti-depressive properties, affect the physical properties and behaviour of the HSPDE4A4-EGFP probe as expressed in stably transfected CHO cells. The results of these experiments show how changes in cellular distribution of the HSPDE4A4-EGFP probe following treatment with a compound can be used to predict or evaluate the biological activity and therapeutic consequences of administering that compound to mammals, especially to humans.

Stably transfected (clonal and non-clonal) CHO cells are allowed to adhere to chambered coverglasses in HAM's F12 medium containing 10% FBS. Once adhered, compounds are added singly to each test chamber, and cells incubated further at 37° C. with 5% CO₂+air. At certain times after addition of the compounds cells are checked on a fluorescence microscope for changes in cellular distribution of GFP fluorescence. Different compounds cause different changes in the pattern of GFP fluorescence in these cells.

The compounds trequinsin (HL-725; 9,10-dimethoxy-2-mesitylimino-3-methyl-3,4,6,7-tetrahydro-2H-pyrimido[6,1-a]isoquinolin-4-one) and etazolate (SQ20009; 1-ethyl-4-[(1-methylethylidene)hydrazino]-1H-pyrazolo[3,4-b]pyridine-5-carboxylic acid ethyl ester, HCl; Calbiochem) both have some inhibitory activity towards PDE4s, although they are not specific inhibitors of these enzymes; IC₅₀ values of trequinsin against PDE4 is 1 μM (Saldou et al.; 1998) and of etazolate is 2 μM (Ahluwalia et al.; 1982). Neither compound causes formation of spots in clonal CHO cells transfected with the HSPDE4A4-EGFP probe.

Denbufylline (BRL 30892, Beecham), a selective PDE4 inhibitor (IC₅₀ 1 μM against PDE4) which is structurally unrelated to Rolipram, produces spots in cells transfected with the HSPDE4A4-EGFP probe (FIG. 9). The spots it produces are indistinguishable from those induced by Rolipram treatment, although it is less effective; only 40% of cells develop spots after 24.5 hours with 10 μM of the compound.

RS 25344 is a specific inhibitor of PDE4 enzymes, also structurally unrelated to Rolipram, which has an IC₅₀ of 0.00028 μM against PDE4s (Saldou et al.; 1998). At 0.03 μM RS25344, spots appeared in about 40% of clonal cells transfected with the HSPDE4A4-EGFP probe after 24.5 hours (FIG. 10a), the spots being indistinguishable from those caused by Rolipram. Under similar conditions, 1 μM of the compound produces very large and bright spots in more than 95% of cells (FIG. 10b).

RP 73401 (also known as Piclamilast; Rhone-Poulenc Rorer), a specific inhibitor of PDE4 enzymes, produces no spots in clonal cells transfected with the HSPDE4A4-EGFP probe when tested over the range 0.3 nM to 3 μM. The IC₅₀ of RP73401 for PDE4 is 0.3 nM (Saldou et al. 1998). Rolipram (2 μM) plus 0.001 μM RP73401 will produce spots in less than 5% of cells (FIG. 11b) 7.5 hours. Under similar conditions, but without RP73401, more than 95% of the same cells respond to 2 μM Rolipram by producing bright spots in their cytoplasm (FIG. 11a). With 0.003 μM RP73401, 2 μM Rolipram is unable to elicit the production of any spots in these cells (FIG. 12). Furthermore, spots take over 4 hours to appear following subsequent replacement of the [Rolipram+RP73401] with 2 μM Rolipram alone, indicating that no anchor proteins accumulate in the combined presence of Rolipram and RP73401.

The Rolipram-like compound Ro-20-1724 (Calbiochem) is a specific inhibitor of PDE4 enzymes with an IC₅₀ of 2 μM (Rubin et al; 1991). At 10 μM Ro-20-1724 spots appear in about 80% of clonal cells transfected with the HSPDE4A4-EGFP probe after 4.5 hours (FIG. 13), the spots being indistinguishable from those caused by Rolipram.

Incubation of non-clonal cells with 500 μM of the non-selective PDE inhibitor IBMX (Sigma Aldrich) causes spots to become visible in only about 5 to 10% of cells after 14 hours incubation. These spots are rather smaller and more numerous within each cell than those formed in the presence of Rolipram (FIG. 14). IBMX is a general inhibitor of all PDEs, and its presence will therefore encourage cAMP levels to rise in treated cells, which is not the case for PDE4 selective inhibitors which leave the activity of other families of PDEs unaffected.

CHO cells transfected with the HSPDE4A4-EGFP probe do not produce spots when treated, in HAM's F12 with or without 10% FBS, with 500 μM theophylline (a general PDE inhibitor), or 100 μM caffeine (a weak and general PDE inhibitor), or 10 μM milrinone (a strong PDE3 inhibitor but also reported to have IC₅₀ for PDE4s of about 10 μM), or 0.5 μM cilostamide (a potent PDE3 inhibitor, IC₅₀ 70 nM), or 100 μM zaprinast (potent PDE5 inhibitor, IC₅₀ 0.4 μM), or 400 μM thalidomide (an anti-inflammatory compound with unspecified mode of action); all these incubations are carefully observed over the period of 1 to 24 hours and none produces spots. Cells treated with 2 μM Rolipram plus either theophylline, caffeine, milrinone, cilostamide or zaprinast (same concentrations as previously, same treatment times and conditions) form the same number and type of bright spots as they do when treated with 2 μM Rolipram alone.

Together, these experiments where PDE inhibitors are simply incubated with cells transfected with the HSPDE4A4-EGFP probe show that the formation in these cells of spots similar to those produced by Rolipram is apparently associated only with the particular sub-class of PDE inhibitors that are potent and specific inhibitors of PDE4. The example shows how screening of compounds for their ability to form spots in these cells can be used to identify PDE4 inhibitors, and that the compounds identified will have similar properties to Rolipram. Further, the example shows how the HSPDE4A4-EGFP-transfected cells can be used to screen for compounds that will prevent Rolipram from forming spots, and that these compounds so identified, such as RP73401, will also be potent and specific inhibitors of PDE4 with certain properties different to those of Rolipram.

### Example 6

Quantitative Assessment of the Effects of Rolipram, RS25344 and Ro 20 1724 on the Cellular Distribution of HSPDE4A4-EGFP Probe in CHO Cells.

This example shows how the number of spots per cell in CHO cells transfected with the HSPDE4A4-EGFP increases in a dose dependent fashion with certain PDE4-specific inhibitors, that this quantity is readily measurable by automated imaging, and that the dose response data from such measurements yield EC₅₀ values that are closely similar to the biological effectiveness of these compounds in therapeutic applications.

FIG. 15 shows dose response curves for spot formation in response to three different PDE4 inhibitors for a stable and clonal CHO cell line transfected with HSPDE4A4-EGFP probe (spot assays). The three inhibitors are Rolipram (▾), RS25344 (▪) and Ro 20-1724 (●). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Data sets are fitted to a 4-parameter Hill equation giving the following EC₅₀ values of 0.34 micromolar for Rolipram, 0.017 micromolar for RS25344 and 3.77 micromolar for Ro 20-1724.

### Example 7

Quantitative Assessment of the Effects of Rolipram on the Cellular Distribution of HSPDE4A4-EGFP, HSPDE4A4-ΔLR2-EGFP and HSPDE4A4-H506N-EGFP Probes in CHO Cells.

This example shows how measurement of the number of spots per cell induced by different concentrations of Rolipram, in CHO cells transfected with various N1 fusions of EGFP to wild-type and mutant forms of HSPDE4A4, can be used to quantify the importance of different amino acids in the primary sequence of the enzyme in the sensitivity of the Rolipram response.

FIG. 16 shows dose response curves for spot formation in response to Rolipram in three stable and clonal cell lines of CHO cells transfected with HSPDE4A4-EGFP (●),

HSPDE4A4-ΔLR2-EGFP (∇) and HSPDE4A4-H506N-EGFP (▾). The number of spots per cell for each concentration is the mean of 4 measurements±sem, where each measurement is itself an average taken from not less than 100 cells. Cells are grown in HAM's F12 medium plus 10% FBS plus various concentrations of Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Data sets are fitted to a 4-parameter Hill equation giving the following EC₅₀ values of 0.34 micromolar Rolipram for the HSPDE4A4-EGFP probe and 0.41 micromolar Rolipram for the HSPDE4A4-ΔLR2-EGFP probe. An EC₅₀ value can not be determined for the HSPDE4A4-H506N-EGFP probe, since the mutation makes it almost unresponsive to Rolipram in this clonal cell line.

The example shows that when 8 amino acid residues comprising the region Ala-313 to Gln-320 in the linker region 2 (LR2) of HSPDE4A4 are deleted, Rolipram-induced spot-formation is not significantly changed relative to that of the wild-type probe. However, mutation of histidine 506 to asparagine (H506N) produces an almost total loss of sensitivity to Rolipram, indicating that this is an essential residue in the protein for transducing the spot formation activated by Rolipram.

### Example 8

Quantitative Assessment of the Effects of Rolipram, RS25344 and Ro 20-1724 on the Cellular Distribution of HSPDE4A4-H506N-EGFP Probe in CHO Cells.

This example shows how the number of spots produced per cell by different PDE4 inhibitors in CHO cells transfected with the HSPDE4A4-H506N-EGFP is useful in discovering compounds that interact with an ensemble of amino acid residues in HSPDE4A4 that are different to those with which Rolipram interacts.

FIG. 17 shows dose-response curves for spot formation in response to three different PDE4 inhibitors for a stable and clonal CHO cell line transfected with the HSPDE4A4-H506N-EGFP probe. The three inhibitors are Rolipram (▾), RS25344 (▪) and Ro 20-1724 (●). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The data set for RS25344 is fitted to a 4-parameter Hill equation giving an EC₅₀ value of 0.125 micromolar. These clonal cells containing the H506N mutant of HSPDE4A4-EGFP are almost unresponsive to the other two inhibitors over the concentrations tested.

The data show that RS25344 is very different to Rolipram and Ro 20-1724 in that it does not require the presence of a histidine at position 506 to effect the production of spots, and this indicates that the RS compound interacts with an ensemble of amino acids that are different to those with which Rolipram and Ro 20-1724 interact. A spot production assay using the HSPDE4A4-H506N-EGFP probe is therefore able to identify other compounds that differ in this respect from Rolipram and Rolipram-like compounds.

### Example 9

Quantitative Assessment of the Effects of RP73401 on the Ability of Rolipram to Produce Spots in CHO Cells Transfected with the HSPDE4A4-EGFP Probe.

This example shows that the spot assay can be run in a competitive way to identify compounds that are specific PDE4 inhibitors which interfere with the ability of Rolipram to form spots, and that the spot assay can be used to quantify the competitive strength of such compounds.

FIG. 18 shows a competitive dose-response curve for Rolipram-induced spot formation in a stable and clonal CHO cell line transfected with HSPDE4A4-EGFP probe. The cells are challenged with a fixed concentration of 2 μM Rolipram and varying concentrations of the specific PDE4 inhibitor RP73401 (Piclamilast). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of RP73401 plus 2 μM Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2.

Approximately 0.003 μM RP73401 is sufficient to inhibit 50% of the spot formation response of these cells that normally results from treatment with 2 μM Rolipram.

### Example 10

Effect upon Rolipram-Induced Distribution of HSPDE4A4-EGFP Following Treatment with 3-Isobutyl-1-Methylxanthine (IBMX) Plus Forskolin Measured as Time Response Profiles in Low and High Throughput.

This example illustrates how the Rolipram-stimulated accumulations, or large spots, of the HSPDE4A4-EGFP probe may be mobilised and dispersed through the action of compounds that increase cAMP levels in transfected cells. Such treatments may be useful as positive controls in screening assays designed to search for novel compounds able to dislocate bound forms of the HSPDE4A4-EGFP probe. The example also illustrates how changes in the distribution of the HSPDE4A4-EGFP probe may be quantified with standard imaging techniques using a fluorescence microscope or a Fluorescence Imaging Plate Reader device (FLIPR, Molecular Devices, Sunnyvale, Calif., USA).

Furthermore, the example provides some evidence for the involvement of cAMP-dependent protein kinase in the dispersal of Rolipram-induced spots. This example also suggests that either assays of PKA activity or of cellular cAMP concentration are useful as secondary screens in conjunction with this PDE-dislocation assay based on the dispersal of Rolipram-induced spots, to rule out compounds causing dispersal through elevation of cAMP and possible activation of PKA.

For the microscopic assessment of spot dispersal, stably transfected (non-clonal) cells transfected with the HSPDE4A4-EGFP probe are allowed to adhere to chambered coverglasses in HAM's F12 medium containing 10% FBS. Once adhered, 2 μM Rolipram is added to the medium, and cells incubated further at 37° C. with 5% CO₂+air until spots form in about 80% of cells (approx. 12 hours or more). Chambered coverglasses are then transferred to a Zeiss 135 inverted microscope for time lapse imaging as described in Example 2, above. At a given point after starting an experiment, a mixture of IBMX and forskolin is added to give final concentrations of 1 mM and 50 μM, respectively. Images are captured at regular intervals to form a time lapse sequence of the response, also as described in Example 2. Individual frames from the sequence are shown in FIGS. 19a, b, c and d. Analysis of the sequence generated the response profiles shown in FIGS. 20a and b. FIG. 20a shows how the fluorescence intensity increases in the cytoplasm with time following application of forskolin plus IBMX; contemporaneously, the fluorescence intensity of each bright spot decreases (FIG. 20b). The average intensity for the entire image does not change significantly over the period of the time lapse sequence (data not shown). These measurements taken together confirm that the bright spots disperse into the cytoplasm under the influence of forskolin plus IBMX, treatments that serve to elevate cytosolic levels of cAMP.

For the FLIPR measurements, clonal CHO cells transfected with the HSPDE4A4-EGFP probe are cultured in a 96-well black microtitre plate (Packard Polyfiltronics ViewPlate-96, Packard Instrument Co.) to near confluency, then treated with 2 μM Rolipram for 24 hours. The plate is washed with KRW buffer plus 2 μM Rolipram. Half the plate is treated with 2 μM H-89 (Calbiochem), a kinase inhibitor especially potent against the cAMP-dependent protein kinases (PKA, IC₅₀ about 50 nM) and incubated a further 20 minutes. The plate is then run in the FLIPR system at 37° C., with addition of IBMX and forskolin to all wells at final concentrations of 500 μM and 50 μM respectively after the first minute. The experiment is continued for a further 45 minutes, readings being taken at intervals of 1 minute. Curves A and B in FIG. 21 represent averages over 8 wells each for responses to IBMX and forskolin, where wells for curve B are treated with compound H-89, and those for curve A are not.

The difference in the levels of response indicates that the inhibitor of PKA has a significant effect on the dispersal of spots induced by elevated cAMP, suggesting a role for PKA in this process.

A PKA-GFP redistribution assay, or SPA-based assay of cAMP, would be useful adjuncts to the Rolipram-induced spot-dispersal assay based on the HSPDE4A4-EGFP probe since they could counterscreen compounds that induced dispersal through elevation of cAMP.

### Example 11

Effect upon Rolipram-Induced Distribution of HSPDE4A4-EGFP Following Treatment with Phorbol-12-Myristate-13-Acetate (PMA) and/or Ionomycin

This example illustrates how the Rolipram-stimulated accumulations, or large spots, of HSPDE4A4-EGFP probe may be mobilised and dispersed through the action of compounds that increase the concentration of cytosolic calcium ([Ca²⁺]_{cyt}) and activate C-type protein kinases (PKC) in transfected cells. Such treatments may be useful as positive controls in screening assays designed to search for novel compounds able to dislocate bound forms of the HSPDE4A4-EGFP probe. This example also suggests that either assays of PKC activity or of changes in [Ca²⁺]_{cyt} are useful as secondary screens in conjunction with this PDE-dislocation assay based on the dispersal of Rolipram-induced spots, to rule out compounds causing dispersal through elevation of [Ca²⁺]_{cyt} and possible activation of PKC.

Non-clonal CHO cells stably transfected with the HSPDE4A4-EGFP probe are allowed to adhere to chambered coverglasses in HAM's F12 medium containing 10% FBS and to grow for 24 hours. After 24 hours, 2 μM Rolipram (Calbiochem) is added to the medium, and cells incubated at 37° C. with 5% CO₂+air for a further 42 hours. Approximately 70% of the cells contain bright spots when viewed on a fluorescence microscope.

Different wells are then treated with fresh HAM's F12 medium plus 10% FBS plus 2 μM Rolipram containing either 0.2% DMSO, 200 nM PMA, 2 μM ionomycin or 200 nM PMA plus 2 μM ionomycin and returned to the incubator for 55 minutes prior to imaging.

FIGS. 22, 23, 24 and 25 show that the number of cells with bright spots is unaffected in the DMSO control (70%), reduced to between 20-40% in the ionomycin treatment and that all spots dissolve entirely in the other two treatments.

In summary, 2 μM ionomycin alone is capable of dispersing Rolipram-induced spots of the HSPDE4A4-EGFP probe in FBS-replete cells, but slowly and in an incomplete manner, whereas 200 nM PMA, ±ionomycin, will disperse all spots of HSPDE4A4-EGFP probe rapidly and completely.

A PKC-GFP redistribution assay, and/or assay to detect changes in [Ca²⁺]_{cyt}, for example a fluorescence based assay with a cell permeable Ca²⁺-sensitive probe such as Fura 2-AM or Fluo 3-AM (both available from Molecular Probes, Eugene, Oreg., USA), are useful adjuncts to the Rolipram-induced spot-dispersal assay based on the HSPDE4A4-EGFP probe since they rule out compounds that induce dispersal through elevation of [Ca²⁺]_{cyt} and/or activation of PKC.

### Example 12

Effect upon Rolipram-Induced Distribution of HSPDE4A4-EGFP Following Treatment with Phorbol-12-Myristate-13-Acetate (PMA) Plus Ionomycin within Serum-Depleted Cells

This example illustrates that in transfected cells which are serum depleted, the Rolipram-stimulated accumulations, or large spots, of the HSPDE4A4-EGFP probe resist the mobilisation or dispersal that is normally elicited by compounds which either increase the concentration of cytosolic calcium ([Ca²⁺]_{cyt}) and/or activate C-type protein kinases (PKC). This example demonstrates that accumulation and dispersal of the HSPDE4A4-EGFP probe involves yet another control switch in addition to the Rolipram-, cAMP- and [PMA±ionomycin]-sensitive behaviours described in the preceding examples. However, this switch affects only the behaviour governed by [PMA±ionomycin]. As such, the system has considerable in-built complexity, analysis of which in a drug-screening setting demands assays of the highest information content, and secondary screens able to unambiguously identify compounds with the desired mode of action.

Clonal CHO cells stably transfected with the HSPDE4A4-EGFP probe are allowed to adhere to chambered coverglasses in HAM's F12 medium containing 10% FBS and to grow for 5 days without change of medium, which by this time is serum-depleted. After 4 days, 2 μM Rolipram is added to the same medium, and cells incubated at 37° C. with 5% CO₂+air for a further 22 hours. Approximately 95% of the cells contain bright spots when viewed on a fluorescence microscope.

Cells are then washed to KRW buffer, with no added FBS but containing 2 μM Rolipram. Individual wells are then treated with either 50 μM forskolin plus 500 μM IBMX or 200 nM PMA plus 2 μM ionomycin. Spots started to disperse in cells treated with forskolin plus IBMX within 10 to 20 minutes (FIGS. 26a and 26b). In cells treated with PMA plus ionomycin there is little or no change in the number or size of spots present in the cells, even after 40 minutes (FIGS. 27a and 27b)

In summary, Rolipram-induced spots of probe HSPDE4A4-EGFP resist dispersal by agents that should activate PKC and increase [Ca²⁺]_{cyt} when cells are depleted of certain substances normally found in foetal bovine serum. Spots in serum depleted cells remain sensitive to dispersal by agents that increase cAMP. Run as a primary screen for compounds that disperse Rolipram-induced spots, the assay does not require a counterscreen for PMA-like compounds or ones that increase [Ca²⁺]_{cyt} if the cells are serum depleted, as when grown in the same medium for 5 days, without addition of fresh serum, in the manner described in this example. A secondary screen for agents that act in the same manner as [IBMX+forskolin] remains a useful adjunct to such a spot disappearance assay, for example a cell-based screen for increased cAMP.

### Example 13

Description of Treatments Found to Cause Reappearance of Spots in CHO Cells Transfected with HSPDE4A4-EGFP from which Spots Have been Cleared by the Removal of Rolipram, and Use of the HSPDE4A4-EGFP Probe in an Assay to Identify Compounds which Inhibit the Reappearance of Bright Cytoplasmic Spots under Such Conditions.

This example describes conditions found to activate the reappearance of Rolipram-like spots in cells that have previously been treated with Rolipram, but then cleared of spots by removal of Rolipram. The example further shows how the reappearance of spots in cells given these appropriate conditions is sensitive to the presence of thalidomide, and therefore how such an assay can be used to screen for compounds with similar properties.

First, CHO cells stably transfected with probe HSPDE4A4-EGFP are grown in HAM's F12 medium with 10% FBS, and with 2 micromolar Rolipram for 15.5 hours. Bright spots are present in more than 95% of all cells. Rolipram is then washed from the cells, and fresh HAM's F12+10% FBS is added. Cells are returned to conditions of 37° C.+5% CO₂ in humidified air (i.e. standard incubator conditions). After 150 minutes, all GFP-bright spots disappear from cells. Spots are induced to reappear in these ways:

A) NaCl is added to the cells to increase the final concentration of salt in the medium. The cells in FIGS. 28a, 28b and 28c are all in HAM's F12+10% FBS to which NaCl has been added to increase the concentration of NaCl by 100 mM. Cells in 28c are additionally treated with 5 micromolar SB203580, a specific inhibitor of p38 mitogen activated protein kinases (p38 MAPK). The cells in FIG. 28b are returned to conditions of 37° C.+5% CO₂ in humidified air (i.e. standard incubator conditions), while those in 28a and 28c are chilled to 4° C. After 4 hours of these treatments cells were fixed with 4% formaldehyde pH 7.0 at room temperature for 1 hour, and washed with PBS buffer ready for imaging. Many small GFP-bright spots form in more than 90% of the chilled cells, but of those returned to incubator conditions (FIG. 28b) less than 5% of cells contain spots. Chilled and SB 203580-treated cells (FIG. 28c) contain significantly fewer, but larger, bright spots per cell than those in FIG. 28a. FIG. 30 shows the response of these cells to various amounts of NaCl where the concentration of salt in the medium has been increased by 0 mM, 5 mM, 50 mM or 100 mM. A second group of the same cells are treated similarly but with the addition of 5 μM SB203580. All treatments are then chilled, fixed and then stained with 1 μM Hoechst 33258 in PBS for 10 minutes at 25° C., and washed twice in PBS. Automated images are collected and analysed for the number of spots per cell as described in Example 2. The number of spots per cell increases in a dose-dependent fashion with increasing salt concentration. SB203580 decreases the number of spots per cell. Examination of the images from the SB203580 treatment (e.g. FIG. 28c) suggests that decreased spot number is accompanied by increased spot size.

B) Cells are left at ambient conditions. The cells in FIG. 29a are pre-treated with Rolipram as above, and washed to remove spots as described. They are then left under ambient conditions (normal air, 22 to 25° C.) rather than in a cell incubator, for a period of 4 hours. During this time the medium evaporates by about 20%, and the pH of the medium shifts from pH6.5 to pH8.1 as the CO₂ in the medium equilibrates with ambient conditions. After 4 hours, spots reappear in the cytoplasm which are indistinguishable from those induced by the original Rolipram treatment. As time continues the proportion of cells containing spots increases as does the size of spots in cells. Return of cells to the incubator after 4 to 6 hours under ambient conditions results in complete reversal of this effect.

The cells in FIG. 29b are treated according to the protocol described in (B) above, and also given 400 μM thalidomide at the time of removal of the Rolipram. The thalidomide appears to hasten disappearance of the spots, but also inhibits return of spots under ambient conditions. FIG. 31 is a dose-response curve for this effect, for which a set of cells are treated with a range of thalidomide concentrations at the time of removal of Rolipram. After 4 hours under ambient conditions the cells are fixed with 4% formaldehyde buffer (pH7) for 1 hour, washed with PBS and stained with 1 μM Hoechst 33258 in PBS for 10 minutes at 25° C., and washed twice in PBS. Automated images are collected and analysed for the number of spots per cell as described in Example 2. The data set for this experiment is fitted to a 4-parameter Hill equation (the curve in FIG. 31) indicating an IC₅₀ value of 33 micromolar thalidomide against the reappearance of spots under these conditions.

Thalidomide also inhibits the reappearance of spots under the NaCl treatment protocol. Cells treated with an addition of 100 mM NaCl as described in (A) above produce a mean spot count per cell (±sem) of 0.856±0.195 after 2 hours at 4° C. In similar cells treated with 400 μM thalidomide at the time of removal of Rolipram, the spot count is 0.364±0.047 after 2 hours at 4° C.

Spots do not reappear in CHO cells stably transfected with probe HSPDE4A4-EGFP under ambient or chilled and salt-supplemented conditions unless the cells are pre-treated with Rolipram. These observations allow the reappearance of spots to be used as a procedure to screen for compounds similar to thalidomide which cannot, or do not, form spots themselves in CHO cells transfected with probe HSPDE4A4-EGFP, and cannot or do not compete directly with Rolipram in the prevention of spot formation in these cells. Such compounds may share certain properties and therapeutic uses in common with thalidomide and related compounds, many of which are known to have useful anti-inflammatory properties together with mild to strong inhibitory actions against PDE4 enzymes.

### Example 14

Effect upon Distribution of Probe HSPDE4A4-EGFP Following Treatment with Ionomycin within Serum-Depleted Rolipram-Treated Cells

This example demonstrates yet another behaviour of the Rolipram-treated HSPDE4A4-EGFP probe, which is restricted to cells grown for long periods in serum-depleted media, or starved of serum in KRW buffer This behaviour involves only the fluorescence seen more or less evenly distributed within the cytoplasm of Rolipram-treated cells, and does not involve the large fluorescent accumulations characteristic of these cells. This example provides evidence that more than one component may be involved in anchoring the HSPDE4A4-EGFP probe in Rolipram-treated cells, and that direct or indirect sensitivity to changes in [Ca²⁺]_{cyt} is a characteristic of that component (or components).

Clonal CHO cells stably transfected with the HSPDE4A4-EGFP probe are allowed to adhere to chambered coverglasses in HAM's F12 medium containing 10% FBS and to grow for 4 days without change of medium. Rolipram at 2 μM is added to the medium 32 hours before the cells are used, and incubation continued at 37° C. with 5% CO₂+air. At the end of this time approximately 95% of the cells contain bright spots when viewed on a fluorescence microscope.

Cells are then washed to KRW buffer, with no added FBS but containing 2 μM Rolipram. Individual wells are then treated first with 1 or 2 μM ionomycin, and then a short time later with 50 μM forskolin plus 500 μM IBMX. Within 1 minute of treatment with ionomycin, at either 1 or 2 μM, small spots form in the cytoplasm of the cells (FIGS. 32a and 32b). This occurs in nearly all cells, whether they contain large Rolipram-induced spots or not. Large spots are not affected in any way during the formation of the smaller spots. The small spots will spontaneously disappear within 10 to 20 minutes. Application of forskolin plus IBMX clears them within minutes (FIG. 33a). The larger spots also disperse in time with this treatment, but more slowly (FIG. 33b). The formation of small spots will not occur in serum-replete cells, or in starved cells that then have been given 10% serum for more than 45 minutes. The response does not occur in cells transfected with the HSPDE4A4-EGFP probe if Rolipram is not present. The time course of transient appearance of the small spots of the HSPDE4A4-EGFP probe is consistent with that of the Ca²⁺ transient generally elicited by ionomycin in treated cells.

This example suggests that the Rolipram-inhibited HSPDE4A4-EGFP probe, or some other anchoring component it is attached to in the Rolipram-inhibited state, is Ca²⁺ sensitive. Since there is no obvious effect on the large accumulations, it is also possible that the HSPDE4A4-EGFP probe distributed throughout the cytoplasm is anchored to a different component than that or those found in the large accumulations or spots. The behaviour of the HSPDE4A4-EGFP probe under these conditions may be used to screen for compounds that remove the calcium sensitivity of the enzyme complex, or which simply prevent the formation of these minor spots. Such compounds may be useful in controlling inflammatory responses of pro-inflammatory cells such as eosinophils and other leucocytes.

### Example 15

Redistribution of HSPDE4A1-EGFP Caused by Treatment with Rolipram

This example shows that the HSPDE4A1-EGFP probe redistributes within cells when treated with Rolipram, but in a way that is very different to the behaviour of the HSPDE4A4-EGFP probe. HSPDE4A1-EGFP accumulates as small perinuclear spots in otherwise untreated CHO cells transfected with the plasmid PS461 (FIGS. 1 and 34a). Rolipram induces these spots to disperse into the cytoplasm (FIG. 34b).

The HSPDE4A1-EGFP probe is useful in the search for dislocators of this isoform, and to discover compounds that mimic or antagonise the effect of Rolipram on the probe. Such compounds will likely be therapeutically useful in the treatment of depressive disorders and inflammatory reactions in the central nervous system.

In FIG. 34a the cells are growing in only HAM's F12 medium with 10% FBS; the GFP fluorescence is restricted to bright granule-like spots within the perinuclear cytoplasm of each cell. The spots may be clustered around, in or on the Golgi membranes. In FIG. 34b similar cells to those seen in 34a have been treated with 2 micromolar Rolipram for 2 hours. The majority of GFP-bright spots disappear in all cells under Rolipram treatment, and the cytoplasm becomes generally brighter. Larger spots may not disperse completely in some cells. When Rolipram is washed away, the spots reform within 1.75 hours. Certain other compounds also reduce PDE4A1 spot numbers, and these include Ro 20-1724, RS25344 and to a lesser extent, denbufylline and IBMX, but the latter compound only starts to have an effect after 100 μM. RP73401 does not disperse spots, and it is anticipated that other such compounds with affinity only for the “low affinity binding site” of PDE4s, such as SB207499 or CDP840 (CellTech/Chiroscience) will also fail to disperse spots of PDE4A1.

This example shows that the HSPDE4A1-EGFP probe does not share the same responses or behaviour demonstrated by the HSPDE4A4-EGFP probe. Since the 4A4 and 4A1 probes share much of the same genomic and therefore primary protein sequence, behavioural differences can be ascribed with some confidence to those regions of the two enzymes that differ. Specifically, these are from amino acid 1 to 22 of probe HSPDE4A1-EGFP and from amino acid 1 to 261 of probe HSPDE4A4-EGFP. The remaining primary sequence of these proteins is identical, as coded for in the plasmids described in Example 1 above.

### Example 16

Quantitative Assessment of the Effects of Rolipram, RS25344, Ro 20-1724, Trequinsin and RP73401 on the Cellular Distribution of HSPDE4A1-EGFP Probe in CHO Cells.

This example shows how different PDE4 inhibitors, and one PDE3 inhibitor with some PDE4 inhibitory activity, either affect the distribution of the 4A1 probe in a dose dependent fashion or have no significant effect on the distribution, that this distribution and any change thereof is readily measurable by automated imaging.

FIG. 35 shows dose response curves for spot dispersal in response to three different PDE4 inhibitors for a stable and clonal CHO cell line transfected with HSPDE4A1-EGFP probe. The three inhibitors are Rolipram, RS25344 (▾) and Ro 20-1724 (◯). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formalin buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Estimated EC₅₀ values are 0.35 micromolar for Rolipram, 0.005 micromolar for RS25344 and 3.5 micromolar for Ro 20-1724. These values are very closely similar to the EC₅₀ values each of these compounds has in causing redistribution of HSPDE4A4-EGFP probe in CHO cells, except in such an experiment spots are formed, and not dispersed as here (example 6).

Data in FIG. 36 has also been obtained by automated imaging from CHO cells stably transfected with HSPDE4A1-EGFP probe, but treated with various concentrations of RP73401 (▾), a specific and potent PDE4 inhibitor, and Trequinsin (∇), a PDE3 inhibitor with some action on PDE4. Again, the number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formalin buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. There is no significant spot dispersal over the concentration ranges tested for either compound, in agreement with the lack of agonist activity of these compounds upon CHO cells stably transfected with HSPDE4A4-EGFP probe.

### Example 17

Quantitative Assessment of the Effects of RP73401 Plus Rolipram on the Cellular Distribution of HSPDE4A1-EGFP Probe in CHO Cells.

This example shows that RP73401 can overcome the effect of Rolipram, and prevent the Rolipram-induced disappearance of spots, in a dose dependent fashion. This example describes how antagonists to the Rolipram effect on 4A1 may be found.

FIG. 37 shows a competitive dose response curve for Rolipram-induced spot dispersal in a stable and clonal CHO cell line transfected with. The cells are challenged with a fixed concentration of 3 μM Rolipram and then varying concentrations of the specific PDE4 inhibitor RP73401 (Piclamilast). The number of spots per cell for each concentration of the different inhibitors is the mean of 4 measurements±sem, where each measurement is itself an average taken from not less than 100 cells. Cells are grown in HAM's F12 medium plus 10% FBS plus 3 μM Rolipram for 20 hours. This treatment removes most spots from all cells. Various concentrations of RP73401 are then added and incubation continued for a further 6 hours. When there is sufficient RP73401 compound present to compete against Rolipram, spots re-form within the cells. The process of spot re-appearance in CHO cells expressing the HSPDE4A1-EGFP probe can be measured as little as 60 minutes after addition of the test compound (RP73401 in this example), or as much as 24 hours after addition, if the test compound is sufficiently stable. Alternatively, both Rolipram and the test compound can be added simultaneously to these cells, and incubation continued for a period in the range 1 hour to 24 hours, after which the spot count per cell will again be indicative of possible antagonistic action.

After the test period, cells are fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. In all cases, if increasing concentrations of a test compound give increasing spot count per cell, then that compound is by definition having an antagonistic effect on the action of Rolipram on 4A1. The data in FIG. 37 are fitted to a 4-parameter Hill equation, giving an IC₅₀ of 0.01 μM for RP73401 against 3 μM Rolipram.

### Example 18

Quantitative Assessment of the Effects of SB207499 (Ariflo®) on the Ability of Rolipram and RS25344 to Produce Spots in CHO Cells Transfected either with the HSPDE4A4-EGFP, HSPDE4A4-ΔLR2-EGFP or HSPDE4A4-H506N-EGFP Probes.

This example shows that Ariflo® (SB207499), a specific PDE4 inhibitor with excellent therapeutic properties and minimal side effect profile is able to prevent or reverse the usual spot forming activity of Rolipram or RS25344 in cells transfected with HSPDE4A4-EGFP, HSPDE4A4-ΔLR2-EGFP or HSPDE4A4-H506N-EGFP probes in a dose dependent way. This example demonstrates that the spot assay can be run in a competitive way to identify compounds that are specific PDE4 inhibitors and which interfere with the ability of Rolipram-like compounds to form spots, and that the spot assay can be used to quantify the competitive strength of such compounds.

Ariflo® does not by itself produce spots in any CHO cells transfected with either HSPDE4A4-EGFP, HSPDE4A4-ΔLR2-EGFP or HSPDE4A4-H506N-EGFP probes over the concentration range 30 to 0.01 μM.

FIG. 38 shows competitive dose-response curves for Rolipram- and RS25344-induced spot formation in a stable and clonal CHO cell line transfected with the HSPDE4A4-EGFP probe. The cells are challenged with fixed concentrations of either Rolipram or RS25344 and various concentrations of Ariflo®. Cells are grown in HAM's F12 medium plus 10% FBS plus various concentrations of RP73401 plus 2 μM Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2.

Approximately 3.5 μM SB207499 is sufficient to inhibit 50% of the spot formation response of these cells to 5 μM Rolipram, indicating approximately equal competitive affinity or strengths of the two compounds for the catalytic cleft of the enzyme. A higher concentration of Ariflo®, approximately 20 μM, is needed to produce the same effect against only 0.5 μM of RS25344, confirming the greater affinity of RS25344 for position in the catalytic cleft.

FIG. 39 shows that the affinity of RS25344, as shown by the amount of Ariflo® needed to halve the spot formation response, is the same for the three different variants of the HSPDE4A4 probes used. This results also indicates that H506 is not involved in binding either RS25344 or Ariflo® to the catalytic cleft, nor is the LR2 region important in this regard.

### Example 19

Redistribution of Probe HSPDE4A4catD-EGFP Caused by Rolipram and RS25344 Treatment

This example illustrates how Rolipram and RS25344 affect the physical properties and behaviour of the HSPDE4A4catD-EGFP probe as expressed in stably transfected CHO cells.

Transiently transfected or stably transfected (non-clonal) cells are allowed to adhere to chambered coverglasses in HAM's F12 medium containing 10% FBS. Once adhered, either Rolipram (4-(3-cyclopentyloxy-4-methoxyphenyl)-2-pyrolidone; Calbiochem) or RS25344 is added to the medium, and cells incubated further at 37° C. with 5% CO₂+air. At certain times after addition of Rolipram cells are checked on a fluorescence microscope for changes in cellular distribution of GFP fluorescence.

These experiments show that the general cytoplasmic distribution of cellular fluorescence gradually changes to one consisting of bright concentrations of fluorescence located at several distinct spots within the cytoplasm, some fluorescence remaining more evenly distributed within the rest of the (non nuclear) cytoplasm. A common pattern appears to be the presence of only 2 major accumulations of fluorescence diametrically separated across the nucleus of the cell (FIG. 40). Spots are stable in cells as long as Rolipram or RS25344 continues to be present.

Spots begin to be visible about 6 hours after addition of 1 μM RS25344. The effect is qualitatively similar at concentrations spanning the range 10 μM to 0.1 μM RS25344, 100 μM to 10 μM Rolipram. Pre-treatment of cells with 5 μg/ml cycloheximide prevents formation of spots induced by Rolipram and RS25344, indicating that protein synthesis is a necessary part of spot formation. Once spots have formed, removal of eother compound results in their rapid dissolution, within 60 minutes at 37° C. However, replacement of either Rolipram or RS25344 causes the bright spots to reform, also within 60 minutes. This is more rapid than is seen for de novo production of spots by Rolipram or RS25344 in these cells.

These experiments indicate that the HSPDE4A4catD-EGFP probe responds to Rolipram or RS25344 in qualitatively the same way as the HSPDE4A4-EGFP probe, and shows that a cassette substitution of PDE4 catalytic regions into the PDE4A4 enzyme is a feasible method to search for isoform-specific catalytic inhibitors of PDE4. By extension from the methods described for the uses of HSPDE4A4-EGFP and HSPDE4A1-EGFP probes, the cassette substitution of other PDE4 isoform catalytic regions into these PDE4A probes will allow discovery of isoform-specific catalytic inhibitors which belong either to the Rolipram group of compounds, or to the group of PDE4 inhibitors with low potential for causing emesis in humans (members of this latter group being Ariflo® and RP73401, for example).

FIG. 40 shows the response to 1 μM RS25344 of a population of CHO cells stably transfected with probe HSPDE4A4cat4D-EGFP. Cells have been treated with RS25344 for 32 hours. Many cells in this stable population respond by forming pairs of bright spots in their cytoplasm. FIG. 41 shows CHO cells transiently transfected with HSPDE4A4cat4D-EGFP and treated with 10 μM Rolipram for 26 hours. A fraction of cells in the heterogenous population respond by forming bright spots of fluorescence in their cytoplasm.

### Example 20

Quantitative Assessment of the Effects of RP73401 on the Reapearance of Stress-Induced Spots in CHO Cells Stably Transfected with HSPDE4A4-E222G Probe.

This example describes the behaviour of the HSPDE4A4-E222G probe in a CHO cell clone treated to produce stress-induced spots (see Example 13), but in the presence of various concentrations of RP73401 compound. The example demonstrates (1) that the E222G version of the PDE4A4 probe behaves in the same way as does the EGFP version, namely that PDE4A4 spots reappear under stress conditions, and (2) that the number of spots per cell that reappear under stress conditions is inhibited in a dose-dependent way by RP73401 compound.

CHO cells stably transfected with the HSPDE4A4-E222G probe are grown in HAM's F12 medium with 10% FBS, and with 3 micromolar Rolipram for 20 hours. Bright spots, usually paired, are present in more than 95% of all cells. Rolipram is then washed from the cells, and fresh HAM's F12 (with no additions) is added. Cells are returned to conditions of 37° C.+5% CO₂ in humidified air (i.e. standard incubator conditions). After 4 hours, all GFP-bright spots disappear from cells.

Cells are then treated with various concentrations of RP73401 in HAM's F12 and left at ambient conditions (normal air, 22 to 25° C.) for a period of 3 hours (stress treatment). During this time the medium evaporates by about 15%, and the pH of the medium shifts from pH6.5 to pH8.1 as the CO₂ in the medium equilibrates with ambient conditions. After 3 hours, spots reappear in the cytoplasm. The cells are then fixed with 4% formalin buffer (pH7) for 15 minutes, washed with PBS and stained with 10 μM Hoechst 33258 in PBS for 15 minutes at 25° C., then washed twice in PBS. Automated images are collected and analysed for the number of spots per cell as described in Example 2.

Spots do not reappear in these CHO cells under ambient conditions unless the cells are pre-treated with Rolipram. Spot reappearance behaviour is indistinguishable from that of cells stably transfected with HSPDE4A4-EGFP probe (Example 13).

FIG. 42 shows a dose response curves for spot reappearance under stress treatment. Estimated IC₅₀ value is 0.3 nanomolar for RP73401. This value is equal to the IC₅₀ value determined for inhibition of PDE4 enzyme by this compound (Saldou et al. 1998). This result indicates that in the absence of Rolipram, the action of RP73401 opposes spot formation with a kinetic determined by simple reversible binding of the compound to the catalytic site.

### Example 21

Quantitative Assessment of the Effects of Rolipram on the Cellular Distribution of HSPDE4A1-E222G Probe in CHO Cells.

This example shows how Rolipram affects the distribution of the PDE4A1-E222G probe in a dose dependent fashion, as measured by automated imaging, and that the response of this probe is indistinguishable from that of the HSPDE4A1-EGFP probe.

FIG. 43 shows a dose response curves for spot dispersal in a clonal line of CHO cells stably transfected with the HSPDE4A1-EGFP probe treated with Rolipram. The number of spots per cell for each concentration of Rolipram is the mean of 3 measurements±sem, where each measurement is itself an average taken from not less than 100 cells. Cells are grown in HAM's F12 medium plus 10% FBS plus various concentrations of Rolipram for 25 hours. The cells are then fixed with 4% formalin buffer (pH7) for 15 minutes, washed with PBS and stained with 10 μM Hoechst 33258 in PBS for 15 minutes at 25° C., then washed twice in PBS. Automated images are collected and analysed for the number of spots per cell as described in Example 2. Estimated EC₅₀ value is 0.1 micromolar for Rolipram. This value is very closely similar to the EC₅₀ value determined for the HSPDE4A1-EGFP probe in CHO cells (0.35 micromolar, Example 16).

### FIGURE LEGENDS

FIG. 1

Confocal fluorescence image showing CHO cells stably transfected with probe HSPDE4A1-EGFP growing in HAM's F12 medium with 10% FBS. The transfected cells are a clonal population derived from a single parent cell. GFP fluorescence is almost entirely restricted to bright granule-like spots within the perinuclear cytoplasm of each cell. The probe is not visible in the nuclei of these cells.

FIG. 2

Confocal fluorescence image showing CHO cells stably transfected with probe HSPDE4A4-EGFP growing in HAM's F12 medium with 10% FBS. The transfected cells are a mixed, non-clonal population. GFP fluorescence is more or less evenly distributed throughout the non-nuclear cytoplasm, darker regions within this area are probably mitochondria from which the probe is apparently excluded.

FIG. 3

Confocal fluorescence image showing CHO cells stably transfected with probe HSPDE4A4-ΔLR2-EGFP growing in HAM's F12 medium with 10% FBS. The transfected cells are a clonal population derived from a single parent cell. GFP fluorescence is more or less evenly distributed throughout the non-nuclear cytoplasm, darker regions within this area are probably mitochondria from which the probe is apparently excluded.

FIG. 4

Confocal fluorescence image showing CHO cells stably transfected with probe HSPDE4A4-H506N-EGFP growing in HAM's F12 medium with 10% FBS. The transfected cells are a clonal population derived from a single parent cell. GFP fluorescence is more or less evenly distributed throughout the non-nuclear cytoplasm, darker regions within this area are probably mitochondria from which the probe is apparently excluded.

FIG. 5

Confocal fluorescence image showing CHO cells stably transfected with probe HSPDE4A4-EGFP growing in HAM's F12 medium with 10% FBS plus 2 micromolar Rolipram. The transfected cells are a mixed, non-clonal population, and have been treated with Rolipram for 42 hours. GFP fluorescence concentrates in bright spots in approximately 70% of the cell population.

As a scale to guide, nuclei sizes are generally in the range of 8 to 15 μm (mean of 11 μm s.d. 2.5 μm (n=15)).

FIG. 6

Confocal fluorescence image showing CHO cells stably transfected with probe HSPDE4A4-EGFP growing in HAM's F12 medium without FBS but with 2 micromolar Rolipram. The transfected cells have been derived from a single cell isolated from a non-clonal population. The cells have been treated with Rolipram for 6.7 hours. GFP fluorescence concentrates in bright spots in more than 95% of the cells.

As a scale to guide, nuclei sizes are generally in the range of 8 to 15 μm (mean of 11 μm s.d. 2.5 μm (n=15)).

FIG. 7

Confocal fluorescence image showing CHO cells stably transfected with probe HSPDE4A4-ΔLR2-EGFP growing in HAM's F12 medium with 10% FBS plus 3 micromolar Rolipram. The transfected cells are a clonal population, and have been treated with Rolipram for 23.5 hours. In approximately 90% of the cells GFP fluorescence concentrates in bright spots, which are indistinguishable from those in seen in Rolipram-treated cells transfected with the “wild-type” probe HSPDE4A4-EGFP.

FIG. 8

Confocal fluorescence image showing CHO cells stably transfected with probe HSPDE4A4-H506N-EGFP growing in HAM's F12 medium with 10% FBS plus 100 micromolar Rolipram. The transfected cells are a clonal population, and have been treated with Rolipram for 23.5 hours. In only approximately 15% of the cells GFP fluorescence concentrates in bright spots, which are indistinguishable from those in seen in Rolipram-treated cells transfected with the “wild-type” probe HSPDE4A4-EGFP.

FIG. 9

Confocal fluorescence image showing CHO cells stably transfected with probe HSPDE4A4-EGFP growing in HAM's F12 medium with 10% FBS and 10 micromolar denbufylline (BRL30892). The transfected cells have been derived from a single cell isolated from a non-clonal population. The cells have been treated with denbufylline for 24.5 hours. GFP fluorescence concentrates in bright spots in approximately 40% of the cells.

FIGS. 10a and 10b

Confocal fluorescence images showing CHO cells stably transfected with probe HSPDE4A4-EGFP growing in HAM's F12 medium with 10% FBS and two concentrations of RS 25344 for 24.5 hours:cells in 10a are treated with 0.03 micromolar RS 25344, cells in 10b with 1 micromolar. The transfected cells have been derived from a single cell isolated from a non-clonal population. GFP fluorescence concentrates in bright spots in approximately 40% of the cells in FIG. 10a. In FIG. 10b the accumulations of GFP fluorescence are considerably more massive, and present in more than 95% of cells.

FIGS. 11a, b

Confocal fluorescence images showing CHO cells stably transfected with probe HSPDE4A4-EGFP. The transfected cells are a clonal population. In FIG. 11a the cells are grown in HAM's F12 medium with 10% FBS plus 2 micromolar Rolipram for 6.7 hours. The cells in FIG. 11b have been treated with a combination of Rolipram plus 0.001 micromolar of the specific PDE4 inhibitor RP 73401 for 7.5 hours. RP73401 inhibits the Rolipram-induced production of spots in these CHO cells; GFP fluorescence concentrates in bright spots in less than 5% of the cell population.

FIG. 12

Confocal fluorescence images showing CHO cells stably transfected with probe HSPDE4A4-EGFP. The transfected cells are a clonal population. The cells are grown in HAM's F12 medium with 10% FBS with a combination of 2 micromolar Rolipram plus 0.003 micromolar of the specific PDE4 inhibitor RP 73401 for 7.5 hours. RP73401 inhibits the Rolipram-induced production of spots in these CHO cells; there are no spots formed in any of the cells.

FIG. 13

Wide-field fluorescence image showing CHO cells stably transfected with probe HSPDE4A4-EGFP growing in HAM's F12 medium with 10% FBS plus 10 micromolar of the specific PDE4 inhibitor Ro-20-1724. The transfected cells are a clonal population, and are been treated with Ro-20-1724 for 4.5 hours. GFP fluorescence concentrates in bright spots in approximately 80% of the cell population.

FIG. 14

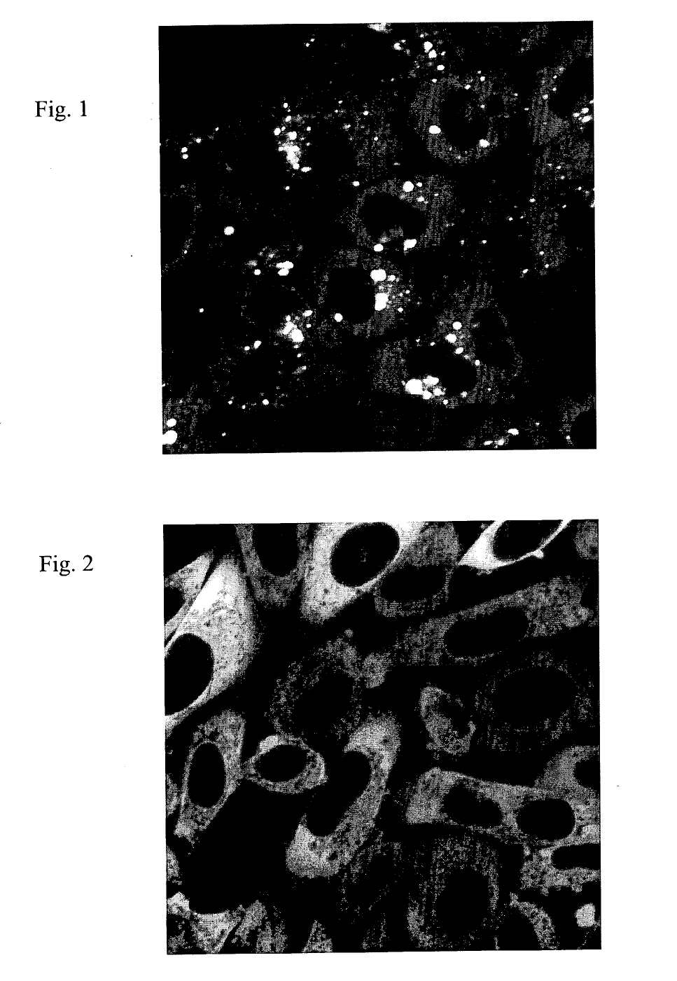

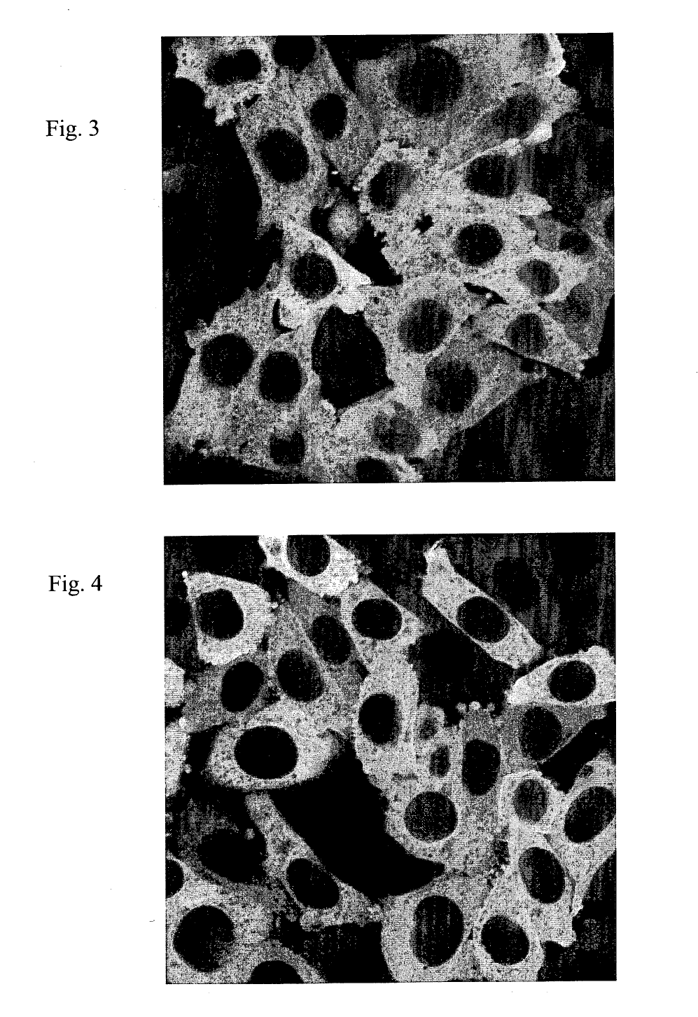

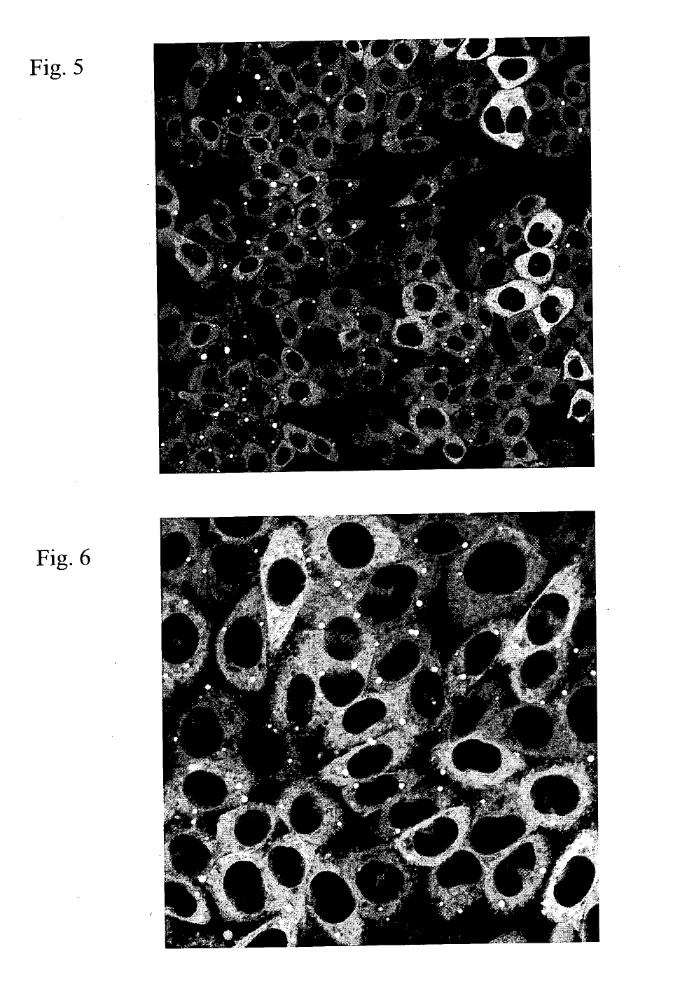

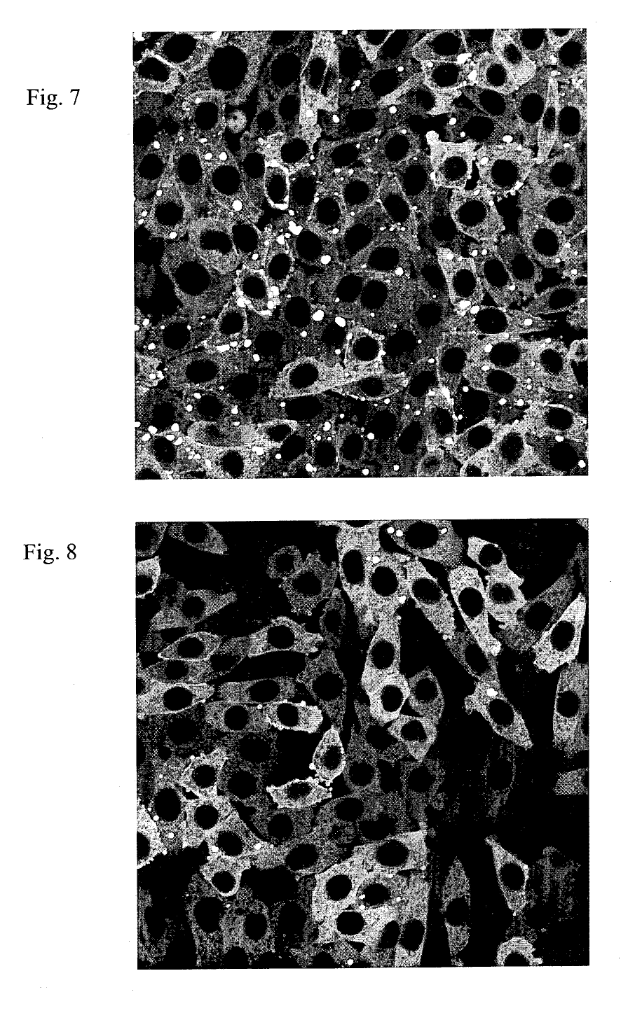

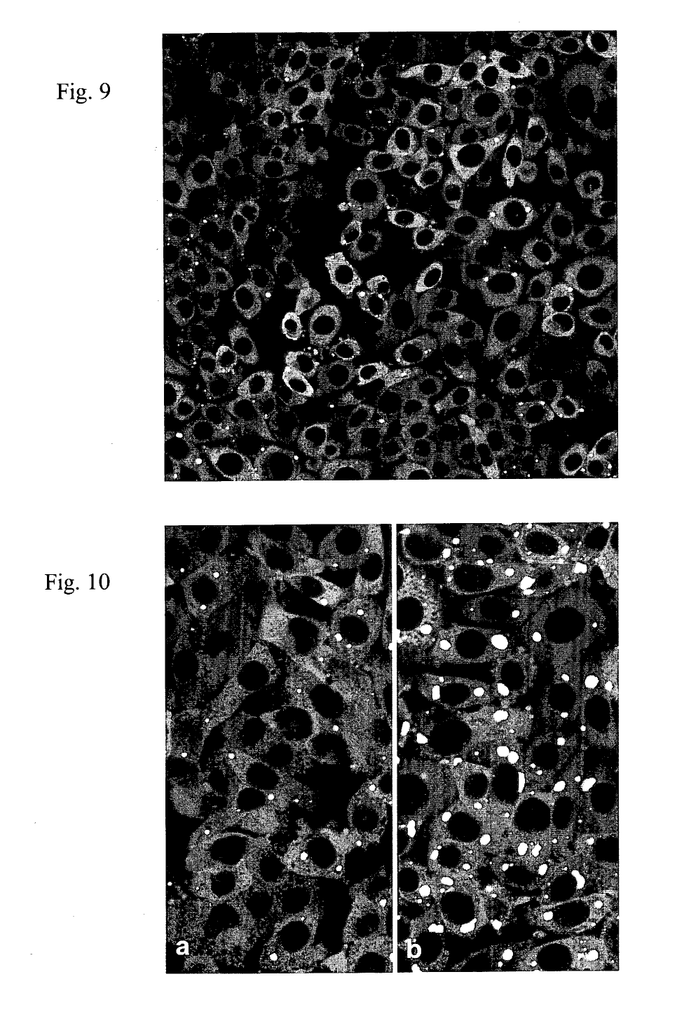

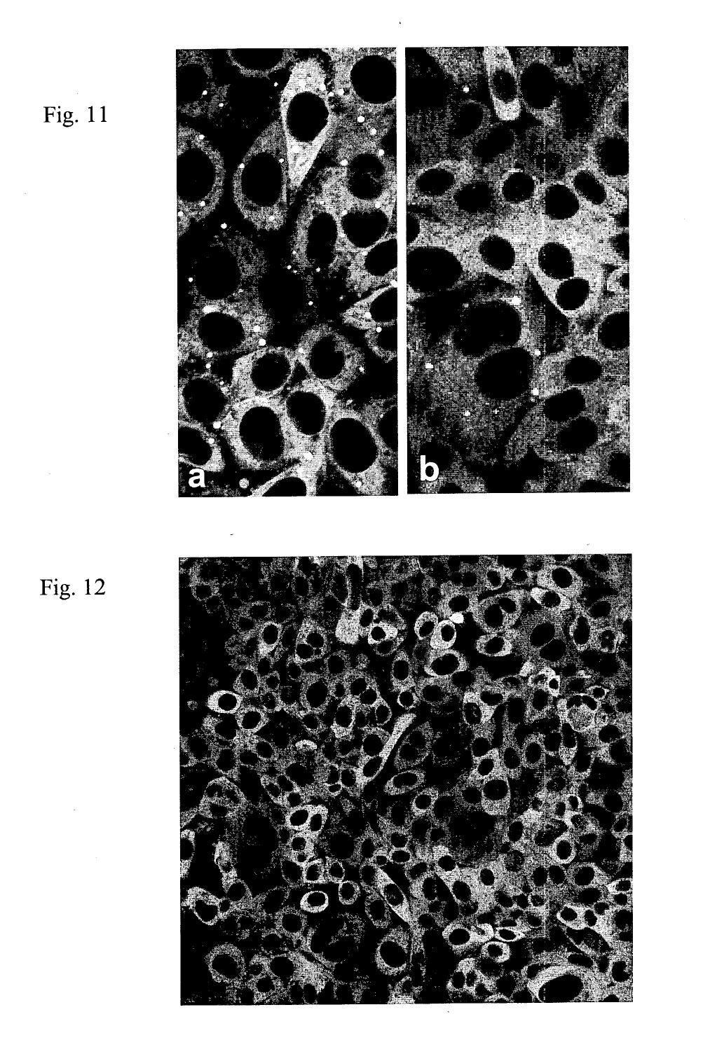

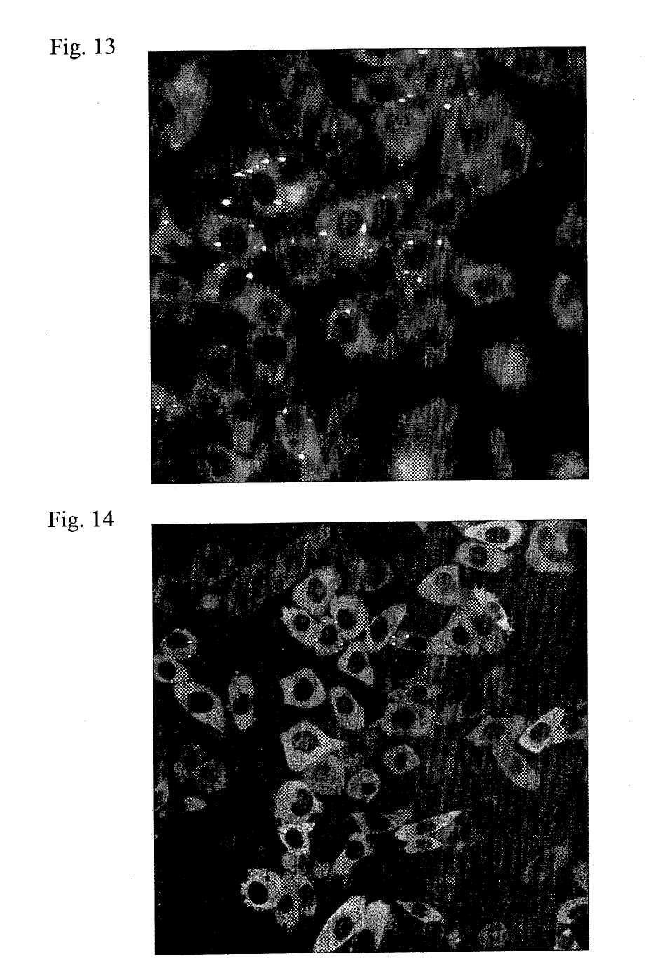

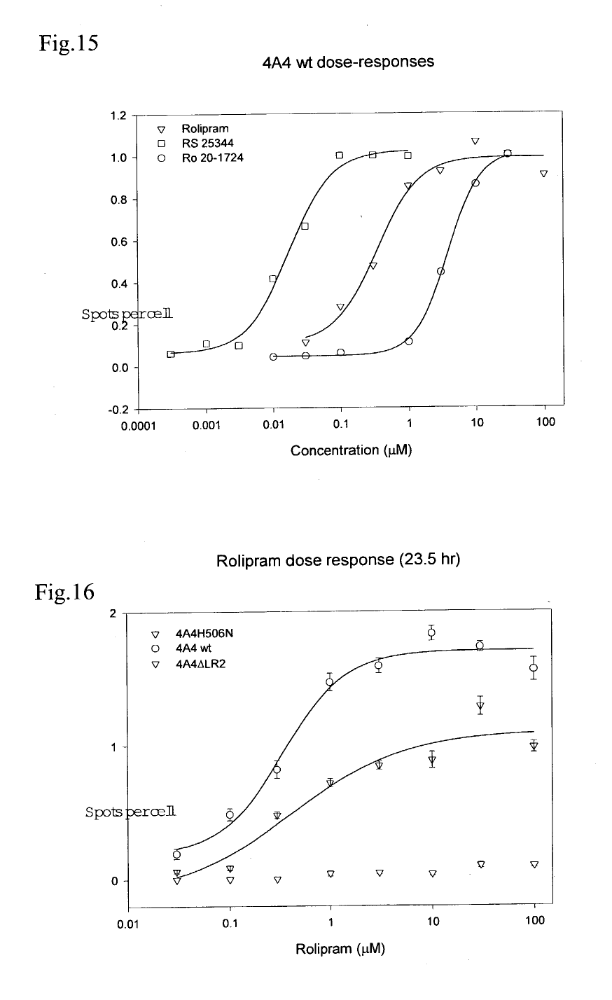

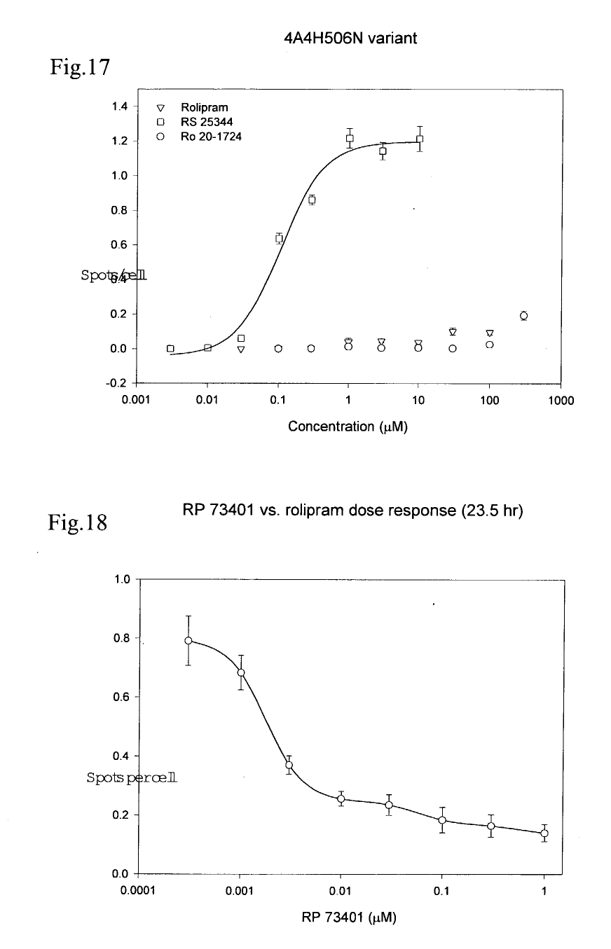

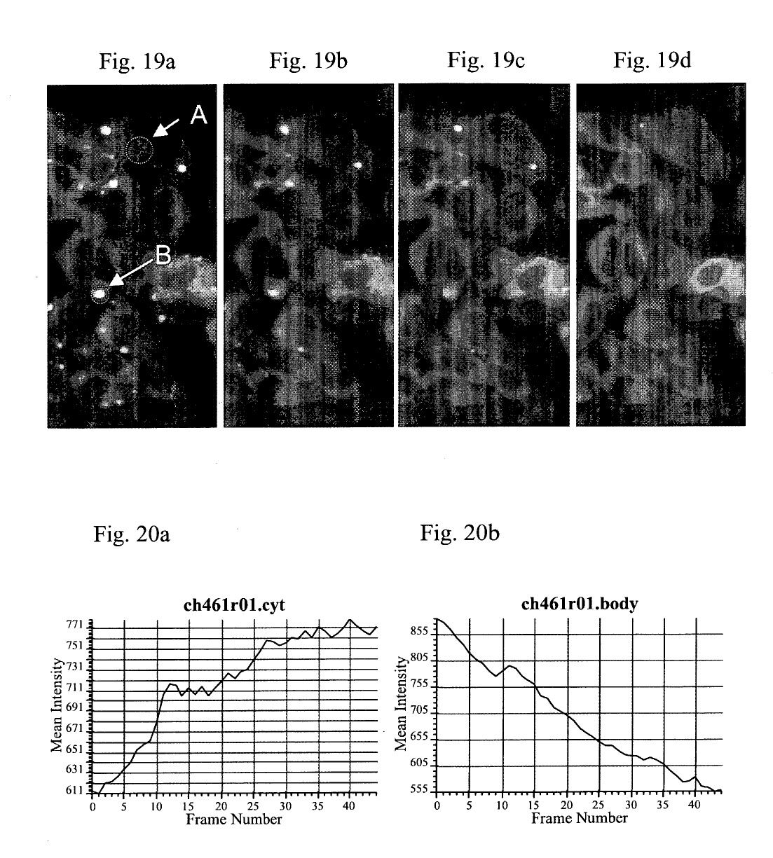

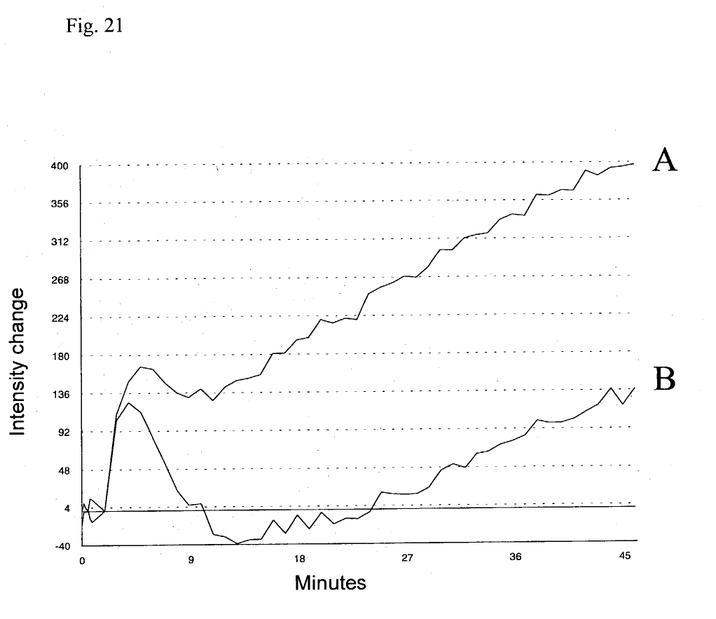

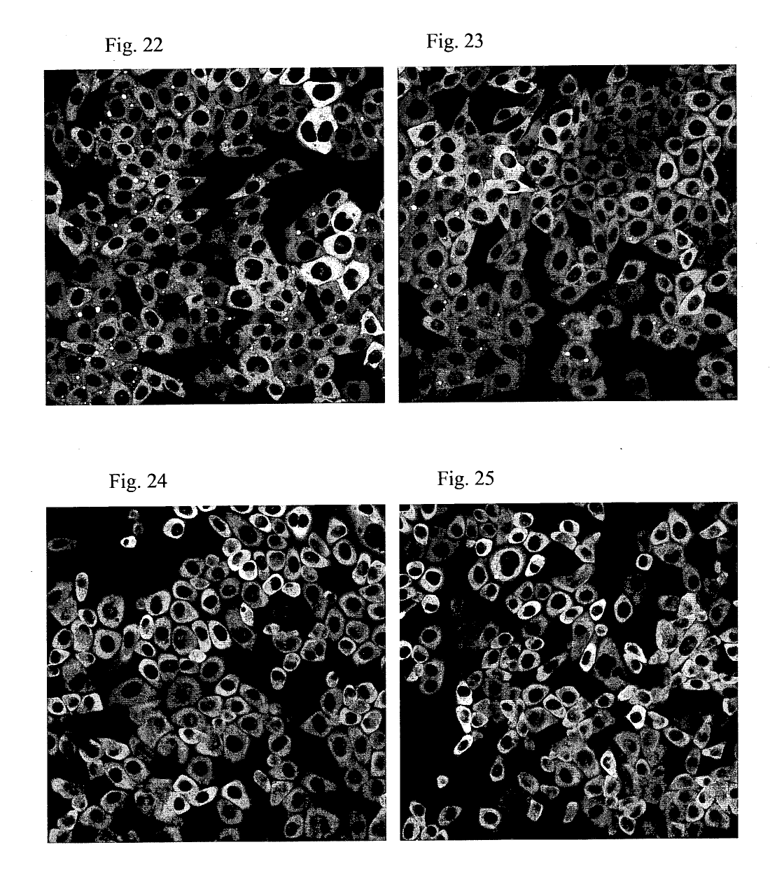

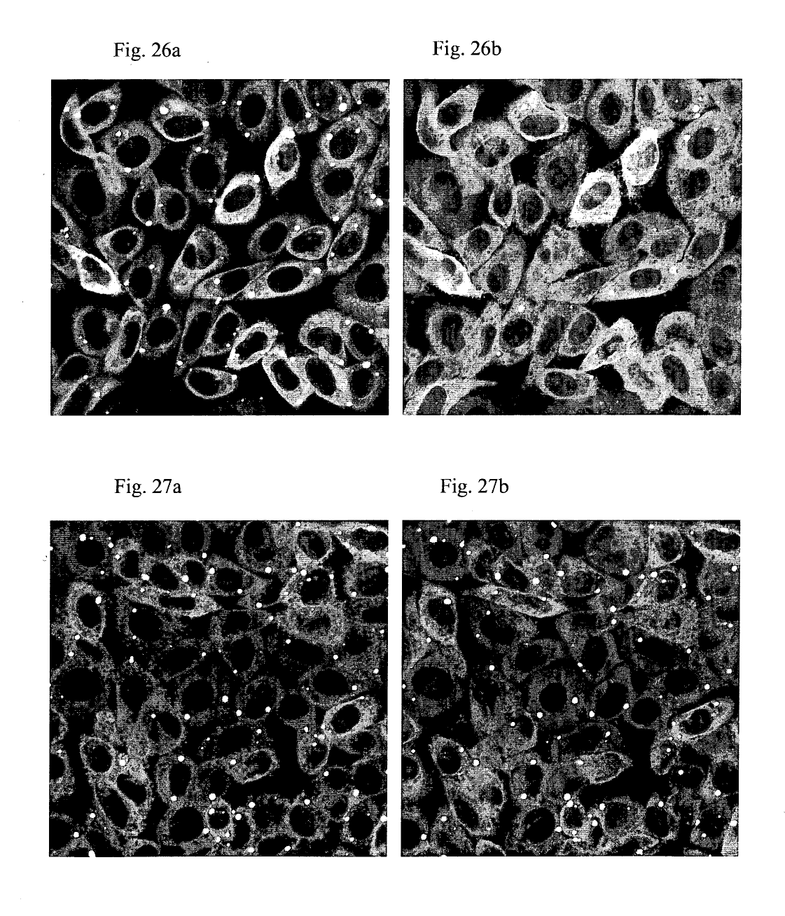

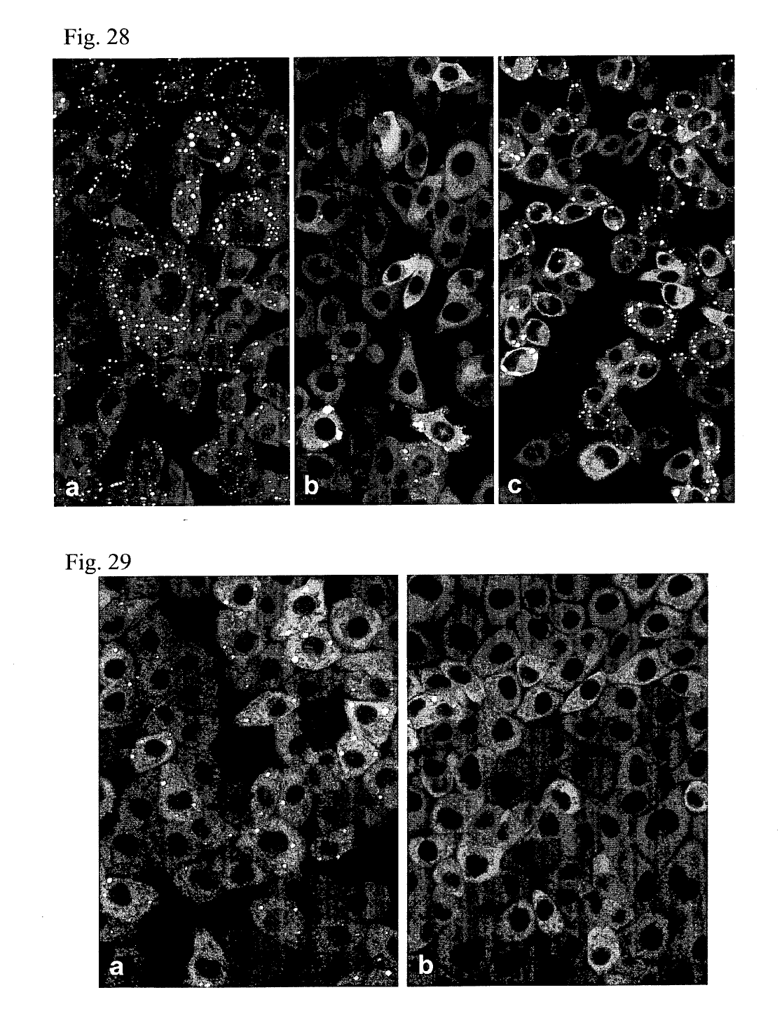

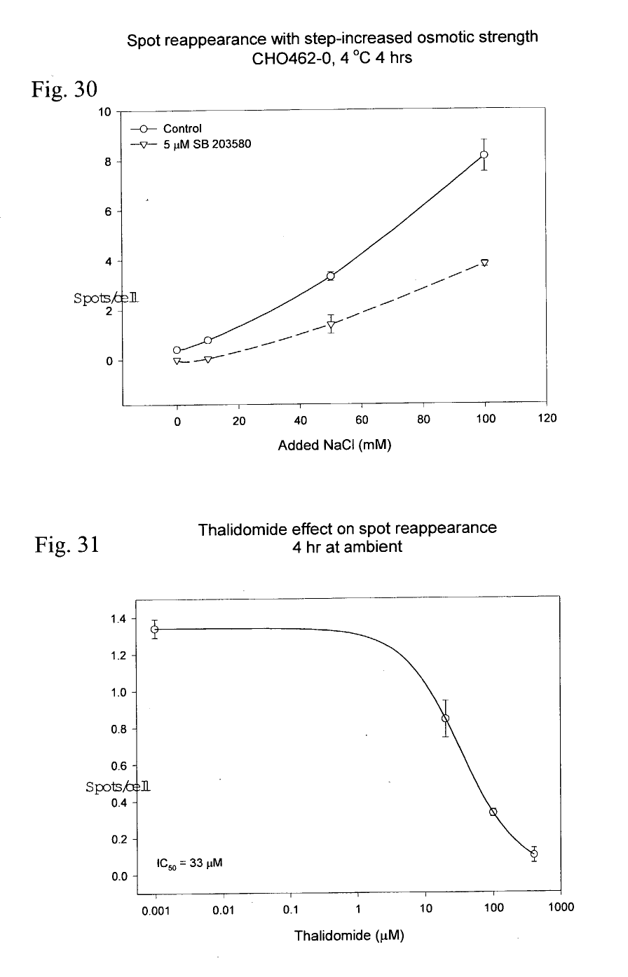

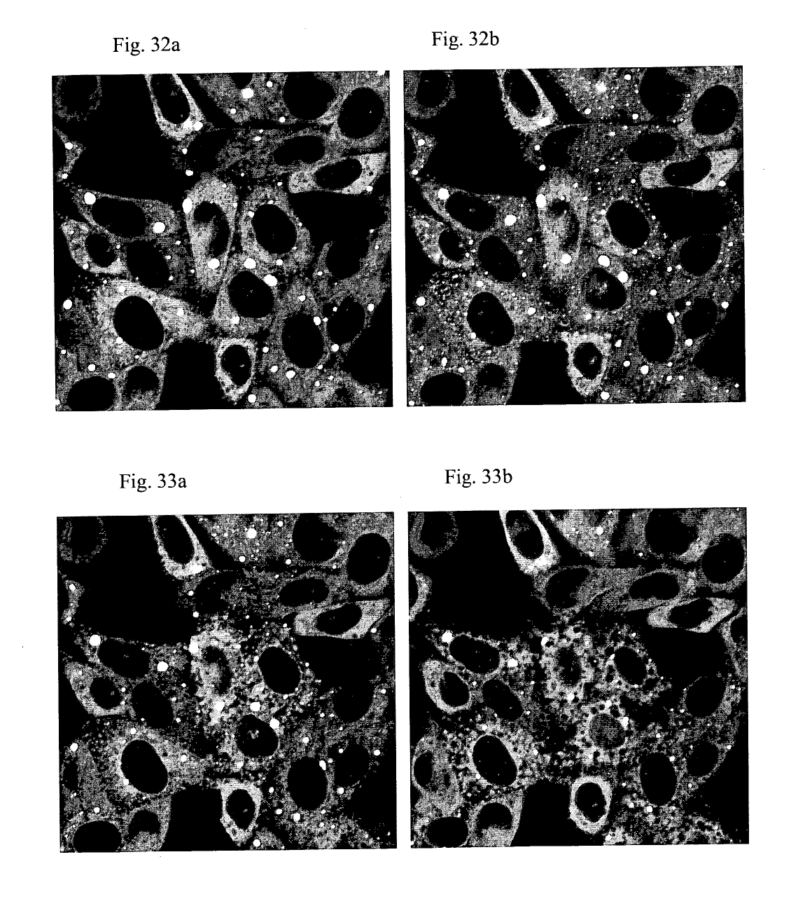

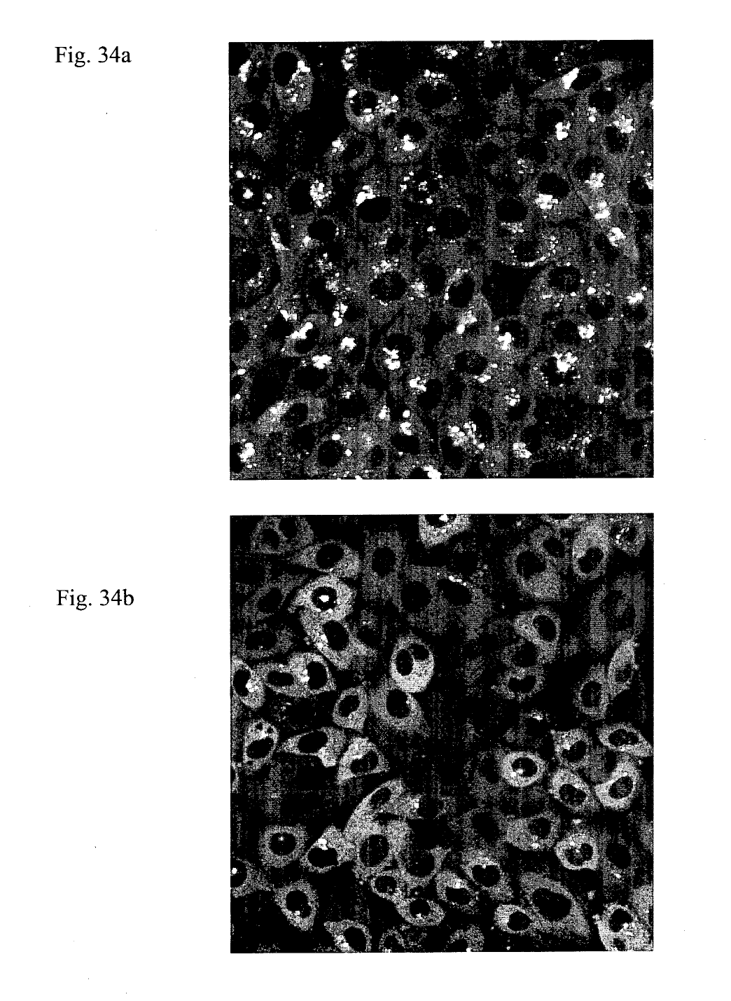

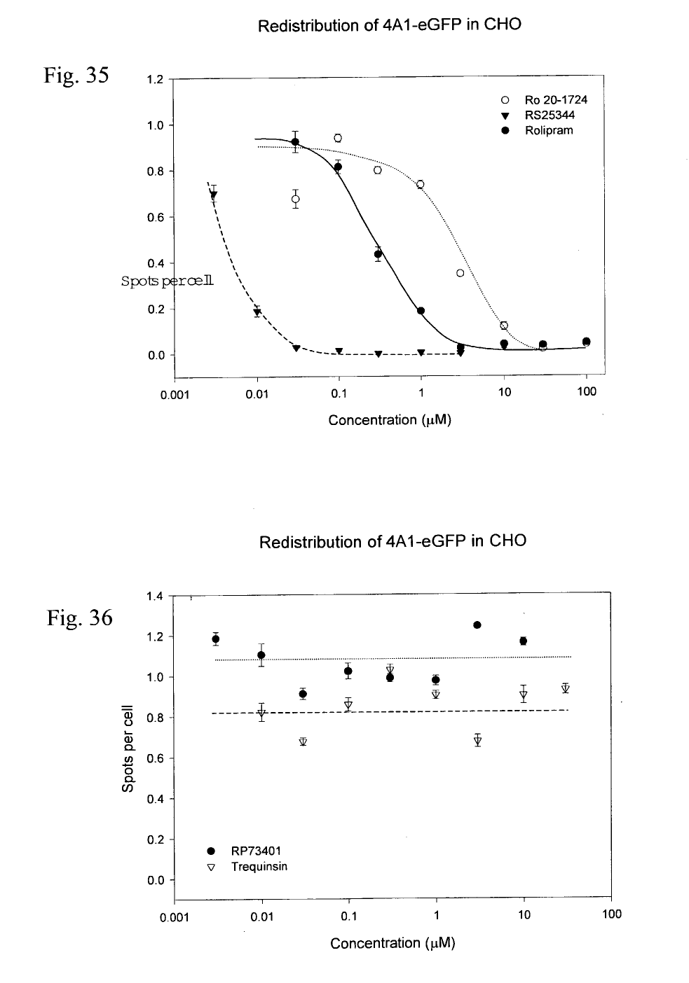

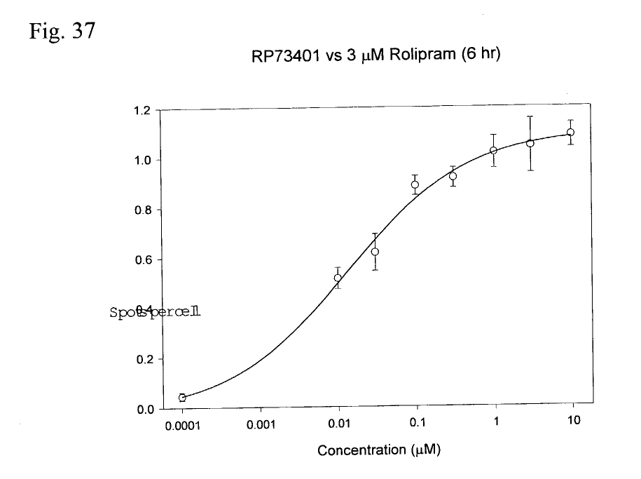

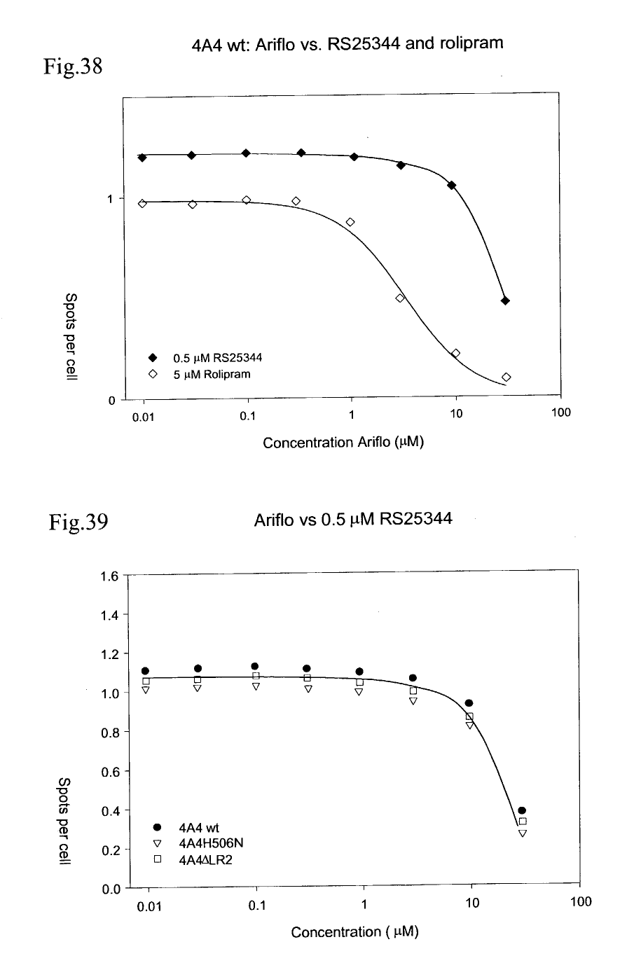

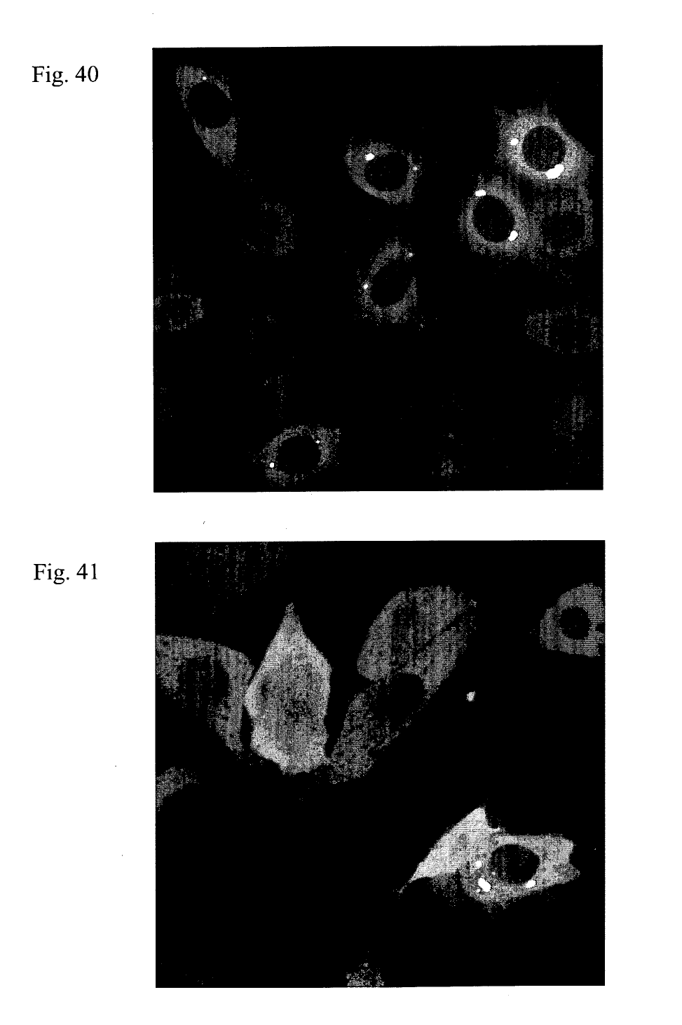

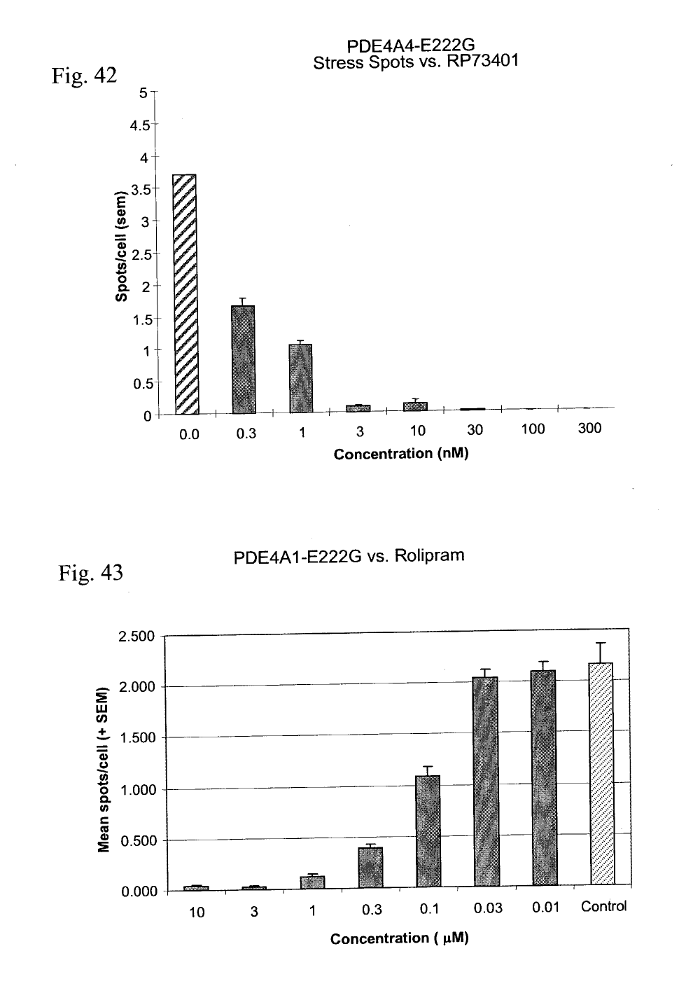

Confocal fluorescence image showing CHO cells stably transfected with probe HSPDE4A4-EGFP growing in HAM's F12 medium with 10% FBS plus 500 micromolar of the general PDE inhibitor IBMX. The transfected cells are a mixed, non-clonal population, and are treated with IBMX for 14 hours. GFP fluorescence forms small bright spots in about 10% of cells. In the remaining cells, the distribution is uniformly cytoplasmic, indistinguishable from untreated cells (FIG. 2). Those cells that contain spots are dissimilar to Rolipram treated cells (FIGS. 5 and 6) in that they each contain more than two major bright spots.

FIG. 15

Dose response curves for spot formation in response to three different PDE4 inhibitors for a stable and clonal CHO cell line transfected with HSPDE4A4-EGFP probe. The three inhibitors are Rolipram (▾), RS25344 (▪) and Ro 20-1724 (●). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Data sets are fitted to a 4-parameter Hill equation giving the following EC₅₀ values of 0.34 micromolar for Rolipram, 0.017 micromolar for RS25344 and 3.77 micromolar for Ro 20-1724.

FIG. 16

Dose response curves for spot formation in response to Rolipram in three stable and clonal cell lines of CHO cells transfected with HSPDE4A4-EGFP (●), HSPDE4A4-ΔLR2-EGFP (∇) and HSPDE4A4-H506N-EGFP (▾). The number of spots per cell for each concentration is the mean of 4 measurements±sem, where each measurement is itself an average taken from not less than 100 cells. Cells are grown in HAM's F12 medium plus 10% FBS plus various concentrations of Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Data sets are fitted to a 4-parameter Hill equation giving the following EC₅₀ values of 0.34 micromolar Rolipram for the HSPDE4A4-EGFP probe and 0.41 micromolar Rolipram for the HSPDE4A4-ΔLR2-EGFP probe. An EC₅₀ value can not be determined for the HSPDE4A4-H506N-EGFP probe, since the mutation makes it almost unresponsive to Rolipram.

FIG. 17

Dose response curves for spot formation in response to three different PDE4 inhibitors for a stable and clonal CHO cell line transfected with the HSPDE4A4-H506N-EGFP probe. The three inhibitors are Rolipram (▾), RS25344 (▪) and Ro 20-1724 (●). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The data set for RS25344 is fitted to a 4-parameter Hill equation giving an EC₅₀ value of 0.125 micromolar. These clonal cells containing the H506N mutant of HSPDE4A4-EGFP are almost unresponsive to the other two inhibitors over the concentrations tested.

FIG. 18

A competitive dose response curve for Rolipram-induced spot formation in a stable and clonal CHO cell line transfected with HSPDE4A4-EGFP probe. The cells are challenged with a fixed concentration of 2 micromolar Rolipram and varying concentrations of the specific PDE4 inhibitor RP73401 (Piclamilast). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of RP73401 plus 2 micromolar Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Approximately 0.003 micromolar RP73401 is sufficient to inhibit 50% of the spot formation response of these cells that normally results from treatment with 2 micromolar Rolipram.

FIGS. 19a to d

The figures show four fluorescence images from a time lapse sequence for non-clonal CHO cells transfected with the HSPDE4A4-EGFP probe. Cells are pre-treated with 2 micromolar Rolipram for 24 hours, then given 50 micromolar forskolin plus 1 millimolar IBMX (all in HAM's F12 medium plus 10% FBS). FIG. 15a is taken immediately before addition of IBMX plus forskolin, FIGS. 15b, c and d at 6, 9 and 24 minutes after that addition. Two regions of interest, marked A and B in FIG. 15a, are used to generate the time profiles shown in FIG. 16a and FIG. 16b respectively (according to the method in Example 2).

FIG. 20a

Time profile derived from a region of interest (ROI) delineating an area of cytoplasm, marked as A in FIG. 15a. IBMX and forskolin are added 2 minutes prior to the start of imaging. The curve is derived by averaging pixel values within the ROI for each image in the sequence. Images are taken at intervals of 30 seconds.

FIG. 20b

Time profile derived from a ROI delineating a single bright spot, marked as B in FIG. 15a. IBMX and forskolin are added 2 minutes prior to the start of imaging. The curve is derived by averaging pixel values within the ROI for each image in the sequence. Images are taken at intervals of 30 seconds.

FIG. 21

This figure shows results from a FLIPR™ (Molecular Devices) 96-well plate reader. The plate contains clonal CHO cells transfected with the HSPDE4A4-EGFP probe that are treated with 2 micromolar Rolipram for 24 hours, then washed to KRW buffer plus 2 micromolar Rolipram just prior to running the experiment. Time traces A and B represent averages over 8 wells each for responses to 500 micromolar IBMX plus 50 micromolar forskolin, where wells for curve B are pre-treated with 2 micromolar compound H-89 for 20 minutes, and those for curve A are not. Curves are normalised and corrected to a buffer+DMSO control. The experiment is run at 37° C., and addition of the test compounds occurs after the first minute. Readings after the addition are made at intervals of 1 minute. The difference in the levels of response indicates that the inhibitor of PKA has a significant effect on the dispersal of spots that is induced by IBMX plus forskolin, suggesting a role for PKA in this process.

FIG. 22

Confocal fluorescence image showing CHO cells stably transfected with probe HSPDE4A4-EGFP growing in HAM's F12 medium with 10% FBS plus 2 micromolar Rolipram. The transfected cells are a mixed, non-clonal population, and are treated with Rolipram for 42 hours, then further treated with fresh HAM's F12 medium with 10% FBS plus 2 micromolar Rolipram plus 0.2% DMSO for 50 minutes. All treatments are under standard incubator conditions. GFP fluorescence remains concentrated in bright spots in approximately 70% of the cell population.

FIG. 23

Confocal fluorescence image showing CHO cells stably transfected with probe HSPDE4A4-EGFP growing in HAM's F12 medium with 10% FBS plus 2 micromolar Rolipram. The transfected cells are a mixed, non-clonal population, and are treated with Rolipram for 42 hours, then further treated with fresh HAM's F12 medium with 10% FBS plus 2 micromolar Rolipram plus 2 micromolar ionomycin for 50 minutes. All treatments are under standard incubator conditions. GFP fluorescence remains concentrated in bright spots in approximately 20 to 40% of the cell population.

FIG. 24

Confocal fluorescence image showing CHO cells stably transfected with probe HSPDE4A4-EGFP growing in HAM's F12 medium with 10% FBS plus 2 micromolar Rolipram. The transfected cells are a mixed, non-clonal population, and are treated with Rolipram for 42 hours, then further treated with fresh HAM's F12 medium with 10% FBS plus 2 micromolar Rolipram plus 200 nM PMA for 50 minutes. All treatments are under standard incubator conditions. GFP fluorescence is no longer concentrated in bright spots in any of the cell population.

FIG. 25

Confocal fluorescence image showing CHO cells stably transfected with probe HSPDE4A4-EGFP growing in HAM's F12 medium with 10% FBS plus 2 micromolar Rolipram. The transfected cells are a mixed, non-clonal population, and have been treated with Rolipram for 42 hours, then further treated with fresh HAM's F12 medium with 10% FBS plus 2 micromolar Rolipram plus 200 nM PMA plus 2 micromolar ionomycin for 50 minutes. All treatments are under standard incubator conditions. GFP fluorescence is no longer concentrated in bright spots in any of the cell population.

FIGS. 26a and 26b

Confocal fluorescence images showing clonal CHO cells stably transfected with probe HSPDE4A4-EGFP in KRW buffer with no FBS plus 2 micromolar Rolipram. The cells are depleted of serum for more than 22 hours. FIG. 26a shows the cells before treatment, FIG. 26b 18 minutes after addition of 50 micromolar forskolin plus 500 micromolar IBMX. This treatment is under ambient conditions on the microscope stage. After 18 minutes, most large spots have dispersed within the cells.

FIGS. 27a and 27b

Confocal fluorescence images showing clonal CHO cells stably transfected with probe HSPDE4A4-EGFP in KRW buffer with no FBS plus 2 micromolar Rolipram. The cells have been depleted of serum for more than 22 hours. FIG. 27a shows the cells before treatment, FIG. 27b 38 minutes after addition of 200 nanomolar PMA plus 2 micromolar ionomycin. This treatment is under ambient conditions on the microscope stage. There is no significant dispersal of the large fluorescent spots under this protocol.

FIGS. 28a, 28b, 28c

Confocal fluorescence images showing CHO cells stably transfected with probe HSPDE4A4-EGFP. The transfected cells have been derived from a single cell isolated from a non-clonal population. These cells are grown in HAM's F12 medium with 10% FBS, and with 2 micromolar Rolipram for 15.5 hours. Bright spots are present in more than 95% of all cells. Rolipram is then washed from the cells, and fresh HAM's F12+10% FBS is added. After 150 minutes all GFP-bright spots disappear from cells. A volume of 1 molar NaCl is then added to the cells to increase the final concentration of salt in the medium by 100 mM. The cells in FIG. 28c are additionally treated with 5 micromolar SB203580, a specific inhibitor of p38 mitogen activated protein kinases (p38 MAPK). The cells in FIG. 28b are returned to conditions of 37° C.+5% CO₂ in humidified air (i.e. standard incubator conditions), while those in 28a and 28c are chilled to 4° C. After 4 hours of these treatments cells were fixed with 4% formaldehyde pH 7.0 at room temperature for 1 hour, and washed with PBS buffer ready for imaging. Many small GFP-bright spots form in more than 90% of the chilled cells, but of those returned to incubator conditions (FIG. 28b) less than 5% of cells contain spots. Chilled and SB 203580-treated cells (FIG. 28c) contain significantly fewer, but larger, bright spots per cell than those in FIG. 28a.

FIGS. 29a, 29b

Confocal fluorescence images showing CHO cells stably transfected with probe HSPDE4A4-EGFP. The transfected cells have been derived from a single cell isolated from a non-clonal population. These cells are grown in HAM's F12 medium without FBS, but with 2 micromolar Rolipram for 12 hours. Bright spots are present in more than 95% of all cells. Rolipram is then washed from the cells, and fresh HAM's F12 (no FBS) added.

The cells in FIG. 29b are additionally treated with 400 micromolar thalidomide. Cells are returned to conditions of 37° C.+5% CO₂ in humidified air (i.e. standard incubator conditions). After 60 minutes all GFP-bright spots disappear from cells. The coverglass chambers or 96-well plates containing the cells are then left at ambient room conditions for a further 4 hours to cool during which the growth medium evaporates by about 20%. During this time GFP-bright spots reappear in about 50% of the cells which are not treated with thalidomide (FIG. 29a). Spots reappear in less than 5% of cells under these conditions in the presence of 400 micromolar thalidomide (FIG. 29b).

FIG. 30

Dose response curves for spot reappearance in response to different concentrations of added NaCl for a stable and clonal CHO cell line transfected with HSPDE4A4-EGFP probe. The number of spots per cell for each concentration of NaCl is the mean of 2 measurements±sem, where each measurement is itself an average taken from not less than 100 cells. These cells are grown in HAM's F12 medium with 10% FBS, plus 2 micromolar Rolipram for 15.5 hours. Bright spots are present in more than 95% of all cells. Rolipram is then washed from the cells, and fresh HAM's F12+10% FBS is added. Cells are returned to conditions of 37° C.+5% CO₂ in humidified air (i.e. standard incubator conditions). After 150 minutes all GFP-bright spots disappear from cells. Various amounts of 1 molar NaCl is then added to different populations of cells to increase the final concentration of salt in the medium by 0 mM, 5 mM, 50 mM or 100 mM. Another group of cells are treated similarly but with the addition of 5 micromolar SB203580, an inhibitor of p38 MAPK. All treatments are then chilled to 4° C., in normal air, for 4 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The number of spots per cell increases in a dose-dependent fashion with increasing salt concentration. SB203580 decreases the number of spots per cell. Examination of the images from the SB203580 treatment (e.g. FIG. 28c) suggests that decreased spot number is accompanied by increased spot size.

FIG. 31

A dose response curve for the inhibition of spot reappearance under ambient conditions by thalidomide for a stable and clonal CHO cell line transfected with HSPDE4A4-EGFP probe. The number of spots per cell for each concentration of thalidomide is the mean of 2 measurements±sem, where each measurement is itself an average taken from not less than 100 cells. These cells are grown in HAM's F12 medium with 10% FBS, plus 2 micromolar Rolipram for 12 hours. Bright spots are present in more than 95% of all cells. Rolipram is then washed from the cells, and fresh HAM's F12+10% FBS is added together with different concentrations of thalidomide. Cells are returned to conditions of 37° C.+5% CO₂ in humidified air (i.e. standard incubator conditions). After 60 minutes all GFP-bright spots disappear from cells. The coverglass chambers (or 96-well plates) containing the cells are then left at ambient room conditions for a further 4 hours to cool during which the growth medium evaporates by about 20%. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The data set is fitted to a 4-parameter Hill equation giving an IC₅₀ value of 33 micromolar thalidomide under these conditions.

FIGS. 32a and 32b

Confocal fluorescence images showing clonal CHO cells stably transfected with probe HSPDE4A4-EGFP treated with 2 micromolar Rolipram. The cells are depleted of serum then washed to KRW buffer with no added FBS for over 3 hours. FIG. 32a shows the cells before treatment, FIG. 32b 3 minutes after addition of 2 micromolar ionomycin. Very many smaller spots appear in the cytoplasm, without changing the size or number of the larger Rolipram-induced spots.

FIGS. 33a and 33b

These images continue from the treatment shown in FIGS. 32a and 32b. The cells are further treated with 50 micromolar forskolin plus 500 micromolar IBMX 7 minutes prior to the image shown in FIG. 33a. There is significant dispersal of the smaller spots by this time. By 24 minutes after forskolin and IBMX treatment (FIG. 33b), the larger spots are beginning to disperse as normal.

FIGS. 34a and 34b

Confocal fluorescence images showing CHO cells stably transfected with probe HSPDE4A1-EGFP. Images are recorded at the same microscope settings for direct comparison of intensities. The transfected cells are a clonal population derived from a single parent cell. In FIG. 34a the cells are growing in only HAM's F12 medium with 10% FBS; the GFP fluorescence is restricted to bright granule-like spots within the perinuclear cytoplasm of each cell. In FIG. 34b similar cells to those seen in 34a have been treated with 2 micromolar Rolipram for 2 hours. The majority of GFP-bright spots disappear in all cells under Rolipram treatment, and the cytoplasm becomes generally brighter. Larger spots do not disperse in some cells. When Rolipram is washed away, the spots reform within 1.75 hours.

FIG. 35

Dose response curves for spot dispersal in response to three different PDE4 inhibitors for a stable and clonal CHO cell line transfected with HSPDE4A1-EGFP probe. The three inhibitors are Rolipram (●), RS25344 (▾) and Ro 20-1724 (◯). The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formalin buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Estimated EC₅₀ values are 0.35 micromolar for Rolipram, 0.005 micromolar for RS25344 and 3.5 micromolar for Ro 20-1724.

FIG. 36

Dose response curves for spot dispersal in response to two different PDE inhibitors for a stable and clonal CHO cell line transfected with HSPDE4A1-EGFP probe. The two inhibitors are RP73401 (●), aspecific and potent PDE4 inhibitor, and Trequinsin (∇), a PDE3 inhibitor with some action on PDE4. The number of spots per cell for each concentration of the different inhibitors is the mean of 4 measurements±sem, where each measurement is itself an average taken from not less than 100 cells. Cells are grown in HAM's F12 medium plus 10% FBS plus various concentrations of inhibitors for 23.5 hours. The cells are then fixed with 4% formalin buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. There is no significant spot dispersal over the concentration ranges tested for either compound.

FIG. 37

A competitive dose response curve for Rolipram-induced spot dispersal in a stable and clonal CHO cell line transfected with HSPDE4A1-EGFP probe. The cells are challenged with a fixed concentration of 3 μM Rolipram and then varying concentrations of the specific PDE4 inhibitor RP73401 (Piclamilast). The number of spots per cell for each concentration of the different inhibitors is the mean of 4 measurements±sem, where each measurement is itself an average taken from not less than 100 cells. Cells are grown in HAM's F12 medium plus 10% FBS plus 3 μM Rolipram for 20 hours. Various concentrations of RP73401 are then added and incubation continued for a further 6 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The data are fitted to a 4-parameter Hill equation, giving an IC₅₀ of 0.01 μM for RP73401 against 3 μM Rolipram.

FIG. 38

Competitive dose response curves for Rolipram- and RS25344-induced spot formation in a stable and clonal CHO cell line transfected with HSPDE4A4-EGFP probe. The cells are challenged with a fixed concentration of either 5 micromolar Rolipram (⋄) or 0.5 micromolar RS25344 (♦) and varying concentrations of the specific PDE4 inhibitor SB207499 (Ariflo®). Cells are grown in HAM's F12 medium plus 10% FBS plus the inhibitors for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. The data set for Rolipram is fitted to a 3-parameter Hill equation giving an IC₅₀ value of 3.37 micromolar for SB207499 in this competition.

FIG. 39

Competitive dose response curves for RS25344-induced spot formation in three stable and clonal CHO cell lines separately transfected with HSPDE4A4-EGFP (●), HSPDE4A4-H506N-EGFP (∇) or HSPDE4A4-ΔLR2-EGFP (□) probes. The cells are challenged with a fixed concentration of 0.5 micromolar RS25344 and varying concentrations of the specific PDE4 inhibitor SB207499 (Ariflo®). Cells are grown in HAM's F12 medium plus 10% FBS plus various concentrations of SB207499 plus 2 micromolar Rolipram for 23.5 hours. The cells are then fixed with 4% formaldehyde buffer (pH7) for 1 hour, washed with PBS and stained with 1 μM Hoechst 33258 in PBS for 10 minutes at 25° C., then washed twice in PBS. Automated images are collected and analysed for the number of spots per cell as described in Example 2. Approximately 20 micromolar SB207499 is needed to reduce by 50% the spot formation response to 0.5 micromolar RS25344.

FIG. 40

Confocal fluorescence image shows the response to 1 μM RS25344 of a population of CHO cells stably transfected with probe HSPDE4A4cat4D-EGFP. Cells have been treated with RS25344 for 32 hours. Many cells in this stable population respond by forming pairs of bright spots in their cytoplasm.

FIG. 41

Confocal fluorescence image shows CHO cells transiently transfected with HSPDE4A4cat4D-EGFP and treated with 10 μM Rolipram for 26 hours. A fraction of cells in the heterogenous population respond by forming bright spots of fluorescence in their cytoplasm.

FIG. 42

Shows a dose response curves for spot reappearance under stress treatment in the presence of various concentrations of RP73401 in a clonal line of CHO cells stably transfected with the HSPDE4A4-E222G probe. The number of stress-induced spots per cell for each concentration of RP73401 is the mean of 3 measurements±sem, where each measurement is itself an average taken from not less than 100 cells. Cells are grown in HAM's F12 medium plus 10% FBS plus 3 μM Rolipram for 20 hours.

Rolipram is then washed from the cells, and fresh HAM's F12 (with no additions) is added. Cells are returned to conditions of 37° C.+5% CO₂ in humidified air (i.e. standard incubator conditions). After 4 hours, all GFP-bright spots disappear from cells. Cells are then treated with various concentrations of RP73401 in HAM's F12 and left at ambient conditions (normal air, 22 to 25° C.) for a period of 3 hours (stress treatment). During this time the medium evaporates by about 15%, and the pH of the medium shifts from pH6.5 to pH8.1 as the CO₂ in the medium equilibrates with ambient conditions. After 3 hours, spots reappear in the cytoplasm.

The cells are then fixed with 4% formalin buffer (pH7) for 15 minutes, washed with PBS and stained with 10 μM Hoechst 33258 in PBS for 15 minutes at 25° C., then washed twice in PBS. Automated images are collected and analysed for the number of spots per cell as described in Example 2. Estimated IC₅₀ value is 0.3 nanomolar for RP73401. This value is equal to the IC₅₀ value determined for inhibition of PDE4 enzyme by this compound (Saldou et al. 1998).

FIG. 43

Shows a dose response curves for spot dispersal in a clonal line of CHO cells stably transfected with the HSPDE4A1-EGFP probe treated with Rolipram. The number of spots per cell for each concentration of Rolipram is the mean of 3 measurements±sem, where each measurement is itself an average taken from not less than 100 cells. Cells are grown in HAM's F12 medium plus 10% FBS plus various concentrations of Rolipram for 25 hours. The cells are then fixed with 4% formalin buffer (pH7) for 15 minutes, washed with PBS and stained with 10 μM Hoechst 33258 in PBS for 15 minutes at 25° C., then washed twice in PBS. Automated images are collected and analysed for the number of spots per cell as described in Example 2. Estimated EC₅₀ value is 0.1 micromolar for Rolipram.

[image]

[image]

[image]

[image]

1. A method to determine if a compound is a dislocator of PDE4, comprising the steps of:
testing if the compound removes PDE4-spots, where PDE4-spots may optionally be induced by a Rolipram-like reference compound and
testing if the compound inhibits the catalytic activity of the PDE4;
the compound being a disclocator of PDE4, if the compound removes PDE4-spots and if the compound does not inhibit the catalytic activity of PDE4.

2. A method according to any of the preceding claims, wherein the Rolipram-like reference compound is Rolipram.

3. A method according to any of the preceding claims, wherein the PDE4 is a PDE4A isoform.

4. A method according to any of the preceding claims, wherein the PDE4 is the PDE4A1 isoform.

5. A method according to any of the preceding claims, wherein the PDE4 is the PDE4A4 isoform.

6. A method according to any of the previous claims, comprising the steps of:
testing if the compound removes PDE4A1-spots, and
testing if the compound inhibits the catalytic activity of the PDE4A1;
the compound being a disclocator of PDE4A1, if the compound removes PDE4A1-spots and if the compound does not inhibit the catalytic activity of PDE4A1.

7. A method according to any of the previous claims, comprising the steps of:
testing if the compound removes PDE4A4-spots induced by a Rolipram-like reference compound and
testing if the compound inhibits the catalytic activity of the PDE4A4;
the compound being a disclocator of PDE4A4, if the compound removes PDE4A4-spots and if the compound does not inhibit the catalytic activity of PDE4A4.

8. A method to monitor changes in intracellular distribution of phosphodiesterases (PDEs) in living cells, the method comprising the steps of:
(a) recording the intracellular distribution of the PDE;
(b) adding a Rolipram-like reference compound to the cells in (a) or to similar cells;
(c) recording the intracellular distribution of the PDE in the cells in step (b);
(d) determining the effect on the intracellular distribution of the PDE of the Rolipram-like reference compound by comparing the intracellular distribution recorded in step (a) with the intracellular distribution recorded in step (c).

9. A method according to the previous claim, further comprising the following steps prior to step (a):
(O1) constructing a probe allowing the location of the PDE to be recorded;
(O2) transfecting cells with the constructed probe of step (a1);

10. A method according to the previous claim, wherein the probe is constructed such that the location of the PDE can be recorded continuously.

11. A method to identify a reagent, capable of interfering with the intracellular distribution of PDEs, comprising the method according to any of the preceding claims further comprising the following steps after step (b):
(b1) adding a reagent to the compound treated cells in step (b) or similar cells;
(b2) recording the intracellular distribution of the PDE in the cells in step (b1);
and the method comprising the following steps after step (d):
(d1) determining the effect of the reagent by comparing the intracellular distribution recorded in step (b2) with the intracellular distribution recorded in step (a);
(d2) establish the pharmacology of the reagent by comparing the determined effect in step (d1) with the determined effect in step (d)
a reversal of the effect determined in step (d) to the effect substantially identical to the effect determined in step (a) in step (d1) being indicative of an antagonistic effect of the reagent on the compound with affinity for the catalytic site of the PDE in regards to the change in intracellular distribution.

12. A method according to any of the preceding claims, further comprising the step of:
(e) determining the effect of the reagent on the catalytic activity of the PDE in an assay capable of measuring the catalytic activity of PDEs.

13. A method according to any of the preceding claims, further comprising the following steps after step (b):
(b1) adding a reagent to similar cells of those in step (a);
(b2) recording the intracellular distribution of the PDE in the cells in step (b1);
and the method comprising the following steps after step (d):
(d1) determining the effect of the reagent by comparing the intracellular distribution recorded in step (b2) with the intracellular distribution recorded in step (a);
(d2) establish the pharmacology of the reagent by comparing the determined effect in step (d1) with the determined effect in step (d)
a copy of the effect determined in step (d) to the effect in step (a) in step (d1) being indicative of an agonistic effect of the reagent on the compound with affinity for the catalytic site of the PDE in regards to the change in intracellular distribution.

14. A method according to any the two previous claims, wherein the reagent does not bind to the docking site of the PDE4.

15. A method according to any of the three previous claims, wherein the reagent binds to the catalytic site of the PDE4.

16. A method according to any of the preceding claims, wherein the reagent inhibits the catalytic activity of the PDE.

17. A method according to any of the preceding claims, wherein the reagent is a peptide or a polypeptide.

18. A method according to any of the preceding claims, wherein the reagent is a small molecule.

19. A method according to any of the preceding claims, wherein PDE is a PDE4.

20. A method according to any of the preceding claims, wherein the Rolipram-like reference compound is Rolipram.

21. A method according to any of the preceding claims, wherein the comparison between the effect of the reagent and the effect of the compound is based on a time series of measurements.

22. A method according to any of the preceding claims, wherein the comparison between the effect of the reagent and the effect of the compound is based on an end-point measurement.

23. A pharmaceutical composition comprising a compound, the compound being a dislocator of PDE4, and the pharmaceutical composition having a market authorisation, the market authorisation being based on an application for market authorisation comprising data showing removal of PDE4-spots, optionally induced by a Rolipram-like reference compound, by the compound and lack of inhibition of the catalytic activity of PDE4 by the compound.

24. A pharmaceutical composition according to the previous claim, wherein the PDE4 is a PDE4A isoform.

25. A pharmaceutical composition according to any of the previous claims, wherein PDE4 is the PDE4A1 isoform and wherein the indication on the market authorisation is diseases in the central nervous system.

26. A pharmaceutical composition according to the previous claim, wherein the indication is depression.

27. A pharmaceutical composition according to any of the previous claims, wherein PDE4 is the PDE4A4 isoform and wherein the indication on the market authorisation is inflammatory diseases.

28. A pharmaceutical composition according to the previous claim, wherein the indication is selected from the group consisting of joint inflammation, Crohn's disease, inflammatory bowel disease, respiratory diseases, chronic obstructive pulmonary disease (COPD), including asthma, chronic bronchitis, pulmonary emphysema, endotoxic shock, toxic shock syndrome, systemic lupus erythematosis, psoriasis, bone resorption diseases, reperfusion injury, cancer and HIV infection.

29. A method to determine if a compound is a low emesis PDE4 inhibitor comprising the steps of:
testing if the compound causes PDE4A4-spots induced by a Rolipram-like reference compound to dissolve,
testing if the compound induces re-appearance of PDE4A1-spots in cells exposed to a Rolipram-like reference compound, and
testing if the compound inhibits the catalytic activity of PDE4;
the compound being a low emesis PDE4 inhibitor if the compound removes spots induced by the Rolipram-like reference compound and induces re-appearance of PDE4A1 spots in cells exposed to the Rolipram-like reference compound and if the compound inhibits the catalytic activity of PDE4.

30. A pharmaceutical composition comprising a compound, the compound being a low emesis PDE4 inhibitor, and the pharmaceutical composition having a market authorisation, the market authorisation being based on an application for market authorisation comprising data showing that the compound removes spots induced by the Rolipram-like reference compound, and that the compound induces re-appearance of PDE4A1 spots in cells exposed to the Rolipram-like reference compound, and that the compound inhibits the catalytic activity of PDE4.

31. A pharmaceutical composition according to any of the previous claims, wherein the indication on the market authorisation is inflammatory diseases.

32. A pharmaceutical composition according to the previous claim, wherein the indication is selected from the group consisting of joint inflammation, Crohn's disease, inflammatory bowel disease, respiratory diseases, chronic obstructive pulmonary disease (COPD), including asthma, chronic bronchitis, pulmonary emphysema, endotoxic shock, toxic shock syndrome, systemic lupus erythematosis, psoriasis, bone resorption diseases, reperfusion injury, cancer and HIV.

33. A method for treating asthma in an individual comprising administering to the individual an effective amount of a compound, or a pharmaceutically acceptable salt, ester, amide or prodrug thereof, the compound being capable of reversing the effect of a compound with affinity for the catalytic site of a PDE on the intracellular distribution of the PDE and mimicking the effect of the compound with affinity for the catalytic site of the PDE on the catalytic activity of the PDE.
